(19) United States
(12) Patent Application Publication
(10) Pub. No.: US 2024/0374558 A1
(43) Pub. Date: Nov. 14, 2024

(54) PREPARATION OF PHENETHYLAMINES AND CATHINONES AND STEREOISOMERS THEREOF AND PRECURSORS THEREOF

(71) Applicant: Transcend Therapeutics, Inc., New York, NY (US)

(72) Inventors: Jennifer Louise SCHMIDT, Garden City, NY (US); Martin STOGNIEW, Lakewood Ranch, FL (US)

(73) Assignee: Transcend Therapeutics, Inc., New York, NY (US)

(21) Appl. No.: 18/673,849

(22) Filed: May 24, 2024

Related U.S. Application Data
(63) Continuation of application No. PCT/US23/35399, filed on Oct. 18, 2023.
(60) Provisional application No. 63/417,896, filed on Oct. 20, 2022.
(60) Provisional application No. 63/437,288, filed on Jan. 5, 2023.
(60) Provisional application No. 63/471,432, filed on Jun. 6, 2023.

(51) Int. Cl. A61K 31/36

(52) U.S. Cl. CPC A61K 31/36

(57) ABSTRACT

In one aspect, the present disclosure provides a method of synthesis for methylone HCl, with 3,4-methylenedioxypropiophenone (MDP) as the starting material. In another aspect, the present disclosure provides stereoisomers of methylone. In another aspect, the present disclosure provides phenethylamines or cathinones covalently bound to a chemical moiety in a prodrug form. The presently described prodrug form allows slow/sustained/controlled delivery of the parent phenethylamines or cathinones into the blood system in a manner that would increase the duration of therapeutic efficacy.

### FIELD OF THE INVENTION

The subject matter disclosed generally relates to phenethylamines and cathinones. The subject matter disclosed relates to methods for preparing the cathinone methylone and its stereoisomers, as well as compositions and uses thereof. The subject matter disclosed also relates to phenethylamines or cathinones covalently bound to a chemical moiety in a prodrug form. The presently described technology allows slow/sustained/controlled delivery of the parent phenethylamines or cathinones into the blood system in a manner that increase the duration of therapeutic efficacy, ease of application, patient compliance and/or a combination of these characteristics when administered, in particular, orally. Additionally, the described technology allows gradual release of the parent phenethylamines or cathinones over an extended time period, thereby eliminating spiking of drug levels which lessen cardiovascular stress, addiction/abuse potential and/or other common stimulant side effects associated with psychoactive compounds.

### BACKGROUND OF THE INVENTION

Methylone (3,4-methylenedioxy-N-methylacthinone) belongs to a group of psychoactive active synthetic cathinones known as β-keto amphetamines. It is a synthetic MDMA analog that differs by the presence of a ketone at the benzylic position. First synthesized in 1996, methylone is a recreational street drug. It induces psychostimulant and empathogenic effects similar to MDMA with a mechanism of action that involve the monoaminergic system.

MDMA (3,4-methylenedioxymethamphetamine), commonly known as ecstasy, is a psychoactive drug primarily used for recreational purposes. MDMA acts primarily by increasing the activity of the neurotransmitters serotonin, dopamine and noradrenaline in parts of the brain. In 2017, the United States Food and Drug Administration (FDA) approved limited research on MDMA-assisted psychotherapy for post-traumatic stress disorder (PTSD), with some preliminary evidence that MDMA may facilitate psychotherapy efficacy.

Despite its close structural analogy with MDMA, methylone has distinct pharmacological and functional properties. Methylone has been shown to improve PTSD symptoms in 81% of patients in a clinical case series of 21 individuals. Currently, the only approved treatments for PTSD are the serotonergic antidepressants sertraline and paroxetine, so drugs that show antidepressant-like activity should improve PTSD symptoms. Methylone has the strongest effect possible in the classic preclinical screen of antidepressant activity, the forced swim test. Methylone also shows benefit in a PTSD mouse model, improving fear extinction recall after fear conditioning, which is consistent with a therapeutic response in this test. Together with the clinical case series results, these data strongly support the potential for clinically effective treatment of PTSD.

Methylone consumers have reported a rapid 15-30 minutes onset of action and a short 2-3.5 hours duration. In a prospective observational-naturalistic study (Lourdes et al. (2021) Biology 10:788) comparing healthy volunteers' self-administration of methylone and MDMA, a significant increase in systolic and diastolic blood pressure was observed for both drugs while only methylone was associated with an increase in heart rate. Subjects reported stimulant-like effects starting at 1-hour post-dosing while most of these effects had almost disappeared after 4 hours.

Parent and metabolites analysis from human and rat urine samples showed a similar metabolic pathway for methylone and MDMA. They are both extensively biotransformed by the cytochrome p450 isoform 2D6 which is in line with their rapid kinetic and short duration of action. In rat PK/PD studies, methylone displayed a rapid kinetic with a T_{Max} of 15 minutes and a t_{1/2} of 1-hour (Elmore et al. (2017) Neuropsychopharmacology 42:649). In the same study, it appears that the methylone plasma concentration correlates with locomotor activation.

As an alternative to sustained released formulations, prodrugs have been used to extend the duration of action and reduce the toxicity and/or side effects associated with the initial spiking of drug levels. Examples of such prodrugs can be found in U.S. Pat. No. 7,105,486 and WO 2022/053696 where the amine functionality of d-amphetamine and MDMA is covalently linked to an amino acid to form an amide bond. In the case of d-amphetamine, the resulting L-Lysine conjugated prodrug known as lisdexamfetamine, displayed a longer duration of action of 10-12 hours compared to 3-6 hours for the unconjugated form of d-amphetamine. A more favorable toxicity/tolerability profile has also been reported for lisdexamfetamine compared to the unconjugated form of d-amphetamine and can be attributed, but not limited to: a significant decrease of the prodrug pharmacological activity due to structure modification, a natural gating mechanism at the site of hydrolysis that limits release of the active amphetamine from the prodrug, and a lack of brain permeability of the prodrug.

Amino groups, such as the one found in methylone or MDMA, can be derivatized to different conjugate prodrugs which are characterized by the newly formed functional group and its specific conversion process to liberate the active drug. Examples of conjugated amine prodrugs such as amide prodrugs, peptide or polypeptide prodrugs, carbamate prodrugs, acyloxyalkoxycarbonyl prodrugs, acyloxymethyl prodrugs, phosphoramide prodrugs and phosphoryloxyalkyl prodrugs can be found in Rautio et al. (2018) Nat. Rev. Drug Discov. 17:559.

It is therefore an object of the present invention to provide a psychoactive agent that displays an advantageous pharmacokinetic and/or pharmacodynamic profile for the treatment of CNS disorders such as PTSD.

It is a further object of the invention to provide a psychoactive agent that displays a favorable toxicity and/or tolerability profile for the treatment of CNS disorder such as PTSD.

It is a further object of the invention to provide prodrugs of a phenethylamine such as MDMA or prodrugs of a cathinone such as methylone that can be hydrolyzed after absorption and be converted directly to the therapeutically active form of the parent compounds.

It is a further object of the invention to provide improved methods of synthesizing a psychoactive agent such as methylone.

It is a further object of the invention to provide stereoisomers of a psychoactive agent such as methylone.

It is a further object of the invention to provide pharmaceutical compositions of a psychoactive agent such as methylone.

### SUMMARY OF THE INVENTION

In one aspect, the present disclosure provides a method of synthesis for methylone HCl, comprising the steps of (i) reacting 3,4-methylenedioxypropiophenone (MDP) with copper (II) bromide and potassium bromide in toluene, and removing insoluble copper salts and soluble copper salts upon completion of the reaction, thereby obtaining 2-bromo-3′,4′-(methylenedioxy)propiophenone (MDPBP); (ii) obtaining a solution of MDPBP in methyl isobutyl ketone (MIBK) and adding a 40% aq. methylamine solution to this MDPBP solution; and (iii) obtaining an organic layer from (ii), and adding HCl in isopropyl alcohol to the organic layer, thereby obtaining methylone HCl. In one embodiment, the method further comprises a step of obtaining a solution of methylone HCl in methanol and adding isopropanol to the methylone HCl solution, thereby obtaining a purified methylone HCl.

In another aspect, the present disclosure is directed to pharmaceutical compositions comprising stereoisomers of compounds described herein, such as stereoisomers of methylone. In one embodiment, the pharmaceutical composition comprises substantially pure (R)-methylone and a pharmaceutically acceptable carrier. In another embodiment, the pharmaceutical composition comprises substantially pure (S)-methylone and a pharmaceutically acceptable carrier. In one embodiment, the pharmaceutical composition comprises (R)-methylone in enantiomeric excess relative to (S)-methylone; and a pharmaceutically acceptable carrier. In another embodiment, the pharmaceutical composition comprises (S)-methylone in enantiomeric excess relative to (R)-methylone; and a pharmaceutically acceptable carrier. This disclosure further provides a method of treatment in mammals of, for example, post-traumatic stress disorder (PTSD), anxiety disorder, attention deficit hyperactivity disorder (ADHD), obsessive compulsive disorder (OCD), fibromyalgia, depression, acute stress disorder (ASD), cluster headache, a condition associated with cancer, diminished drive, burn-out, bore-out, migraine, Parkinson's disease, pulmonary hypertension, schizophrenia, an eating disorder, nausea, or vomiting, by the administration of an effective amount of methylone stereoisomers. This disclosure further provides a method of treatment in mammals of, for example, mood disorders, anxiety disorders, personality disorders, fibromyalgia, suicidal ideation, substance use disorders (SUD), eating disorders, Borderline Personality Disorder (BPD) and other personality disorders, obsessive-compulsive disorder (OCD), palliative care/end-of-life anxiety, existential distress, chronic pain syndromes, body dysmorphia, phobias, social anxiety in autistic adults, and sleep regulation, by the administration of an effective amount of methylone stereoisomers.

In another aspect, the present disclosure is directed to pharmaceutical compositions of the compounds described herein, such as methylone, including pharmaceutically acceptable salts of methylone and/or stereoisomers of methylone, and/or isotopologues and isotopomers of methylone, as well as polymorphs and other solid forms of any of the foregoing. In one embodiment, the pharmaceutical compositions of methylone are high-purity pharmaceutical compositions of methylone. In one embodiment, the pharmaceutical compositions of methylone are room temperature stable compositions of methylone. In one embodiment, the pharmaceutical compositions of methylone are not mutagenic and lack mutagenic impurities. In one embodiment, the pharmaceutical compositions of methylone are suitable for use in humans. In one embodiment, the pharmaceutical compositions of methylone are commercial scale pharmaceutical compositions of methylone. This disclosure further provides a method of treatment in mammals of, for example, post-traumatic stress disorder (PTSD), anxiety disorder, attention deficit hyperactivity disorder (ADHD), obsessive compulsive disorder (OCD), fibromyalgia, depression, acute stress disorder (ASD), cluster headache, a condition associated with cancer, diminished drive, burn-out, bore-out, migraine, Parkinson's disease, pulmonary hypertension, schizophrenia, an eating disorder, nausea, or vomiting, by the administration of an effective amount of the pharmaceutical compositions of methylone. This disclosure further provides a method of treatment in mammals of, for example, mood disorders, anxiety disorders, personality disorders, fibromyalgia, suicidal ideation, substance use disorders (SUD), eating disorders, Borderline Personality Disorder (BPD) and other personality disorders, obsessive-compulsive disorder (OCD), palliative care/end-of-life anxiety, existential distress, chronic pain syndromes, body dysmorphia, phobias, social anxiety in autistic adults, and sleep regulation, by the administration of an effective amount of the pharmaceutical compositions of methylone.

In another aspect, the present disclosure is directed to compounds that are phenethylamine or cathinone precursors in a prodrug form. This disclosure also provides a pharmaceutical composition that includes an effective amount of the phenethylamine or cathinone precursor and a pharmaceutically acceptable carrier. This disclosure further provides a method of treatment in mammals of, for example, post-traumatic stress disorder (PTSD), anxiety disorder, attention deficit hyperactivity disorder (ADHD), obsessive compulsive disorder (OCD), fibromyalgia, depression, acute stress disorder (ASD), cluster headache, a condition associated with cancer, diminished drive, burn-out, bore-out, migraine, Parkinson's disease, pulmonary hypertension, schizophrenia, an eating disorder, nausea, or vomiting, by the administration of an effective amount of the phenethylamine or cathinone precursor. This disclosure further provides a method of treatment in mammals of, for example, mood disorders, anxiety disorders, personality disorders, fibromyalgia, suicidal ideation, substance use disorders (SUD), eating disorders, Borderline Personality Disorder (BPD) and other personality disorders, obsessive-compulsive disorder (OCD), palliative care/end-of-life anxiety, existential distress, chronic pain syndromes, body dysmorphia, phobias, social anxiety in autistic adults, and sleep regulation, by the administration of an effective amount of the phenethylamine or cathinone precursor.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIGS. 1A-1D: Methylone improves fear extinction recall in a mouse model of PTSD. (FIG. 1A) Schematic of experimental design. A single CS-US (tone-shock) pairing on day 1 was followed by 6 CS presentations in a novel context (context B). Methylone or saline vehicle was injected 30 min prior to extinction training on day 2. On day 3, the time spent freezing to the CS was quantified. (FIG. 1B) Freezing time during the first cue on day 3 (extinction recall) was significantly reduced by methylone compared to saline (t₍₂₆₎=2.350, p<0.05). (FIG. 1C) The time freezing before vs. during each of 6 cues on day 3 (to control for locomotor effects) are shown. There was a significant cue x drug interaction (F(_{5,130})=2.409, p<0.05). (FIG. 1D) No locomotor changes were observed on day 3 (t₍₂₆₎=1.073, p>0.05). N=12 for methylone group (30 mg/kg, IP, orange diamonds) and N=16 for saline control group (black squares). *p<0.05

FIG. 2: One Methylone Enantiomer ((S)-methylone) Mimics the Rapid and Robust Antidepressant-like Activity of Racemic Methylone in the Rat Forced Swim Test (FST). A single dose of (R)-methylone, (S)-methylone, racemic methylone (all 10 mg/kg, IP) or vehicle was administered as a single dose 30 min before testing in the FST. Immobility for the duration of the test (5 min) is shown as the % time spent immobile (e.g., 60% of 5 min=3 min). ****p<0.0001 vs. Vehicle, N=8-9 per group.

FIG. 3: Dynamic Vapor Sorption (DVS) results of TCL20422 (lot 213220, Form B+Form A mixture), % weight change vs. % RH (top) and weight vs. time (bottom) and shows that this material does not absorb or desorb water.

### DETAILED DESCRIPTION OF THE INVENTION

In one aspect, the present disclosure provides methods of synthesis for methylone HCl, comprising the steps of (i) reacting 3,4-methylenedioxypropiophenone (MDP) with copper (II) bromide and potassium bromide in toluene, and removing insoluble copper salts and soluble copper salts upon completion of the reaction, thereby obtaining 2-bromo-3′,4′-(methylenedioxy)propiophenone (MDPBP); (ii) obtaining a solution of MDPBP in methyl isobutyl ketone (MIBK) and adding a 40% aq. methylamine solution to this MDPBP solution; and (iii) obtaining an organic layer from (ii), and adding HCl in isopropyl alcohol to the organic layer, thereby obtaining methylone HCl. In one embodiment, the reaction in step (i) is carried out at 85-95° C. In one embodiment, the insoluble copper salts are removed by filtering through celite. In one embodiment, the soluble copper salts are removed by washing with ammonium hydroxide. In one embodiment, the solution comprising MDPBP and methylamine in step (ii) is mixed at 30° C. In one embodiment, the HCl in isopropyl alcohol in step (iii) is added to the organic layer at a temperature below 10° C., for example, at a temperature of 0-10° C.

In one embodiment, the method further comprises a step of obtaining a solution of methylone HCl in methanol and adding isopropanol to the methylone HCl solution, thereby obtaining a purified methylone HCl. In one embodiment, the solution comprising methylone HCl and isopropanol is heated to reflux at 65° C. In one embodiment, the solution comprising methylone HCl and isopropanol is kept at 0-10° C. after heated to reflux at 65° C. In one embodiment, the purified methylone HCl is obtained by drying under reduced pressure at 60° C.

In another aspect, the present disclosure is directed to pharmaceutical compositions comprising stereoisomers of compounds described herein, such as stereoisomers of methylone. In one embodiment, the pharmaceutical composition comprises substantially pure (R)-methylone and a pharmaceutically acceptable carrier. In another embodiment, the pharmaceutical composition comprises substantially pure (S)-methylone and a pharmaceutically acceptable carrier. In one embodiment, the pharmaceutical composition comprises (R)-methylone in enantiomeric excess relative to (S)-methylone; and a pharmaceutically acceptable carrier. In another embodiment, the pharmaceutical composition comprises (S)-methylone in enantiomeric excess relative to (R)-methylone; and a pharmaceutically acceptable carrier.

In another aspect, the present disclosure provides phenethylamine or cathinone prodrugs that exhibit advantageous pharmacokinetic properties and a beneficial side effect profile, which renders the compounds provided herein particularly well suitable for therapeutic use.

In one embodiment, provided herein are compounds represented by Formula (I):

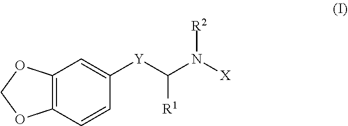

or a pharmaceutically acceptable salt thereof, wherein:
Y is —C(O)— or —CH₂—;
X is independently selected from the group consisting of:
   (a) an amino acid or a peptide,
   (b) —C(O)R³,
   (c) —C(O)OR³,
   (d) —C(O)OCH(R⁴)ORs,
   (e) —CH₂OC(O)R³,
   (f) —P(O)(OH)₂,
   (g) —CH₂OP(O)(OH)₂,
   (h) —C(O)(CH₂)ₙZ^{a}R⁵,

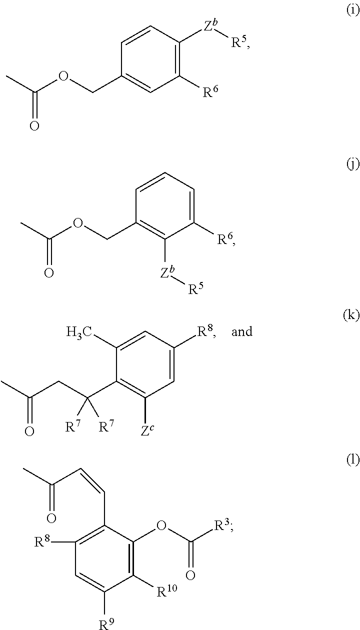

wherein:
n is 3 or 4;
R¹ and R² are each independently —C₁₋₆alkyl or —C₃₋₆cycloalkyl;
R³ is selected from the group consisting of: —C₁₋₆alkyl, —C₃₋₆cycloalkyl, —C₁₋₆haloalkyl, aryl, heteroaryl,

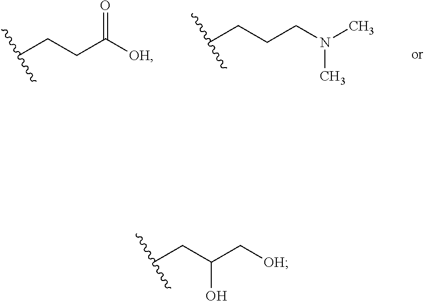

R⁴, R⁷, R⁸, R⁹ and R¹⁰ are each independently H, —C₁₋₆alkyl or —C₃₋₆cycloalkyl;
R⁵ is selected from the group consisting of: —C(O)R³, —C(O)OR³, —P(O)OR¹¹OR¹²), an amino acid, and a peptide;
Z^{a} and Z^{b} are each independently 0 or NR⁴;
Z^{c} is selected from: OC(O)R³ or OP(O)(OR⁴)₂;
R⁶ is selected from the group consisting of: H, —C₁₋₆alkyl, —C₃₋₆cycloalkyl, alkoxy, amino, nitro, halo, cyano, —OH and CF₃;
R¹¹ and R¹² are each independently H, —C₁₋₆alkyl, —C₁₋₆heteroalkyl, —C₃₋₆cycloalkyl, —C₁₋₆haloalkyl, aryl or heteroaryl, wherein —C₁₋₆alkyl, —C₁₋₆heteroalkyl, —C₃₋₆cycloalkyl, —C₁₋₆haloalkyl, aryl or heteroaryl is unsubstituted or substituted with one or more —C₁₋₆alkyl, —C₁₋₆heteroalkyl, —C₃₋₆cycloalkyl, —C₁₋₆haloalkyl, aryl or heteroaryl.

In some embodiments of the foregoing compounds, R¹ and R² are each independently methyl or ethyl. In some embodiments, R⁴ is H or methyl. In some embodiments, Z^{a}, is NH. In some embodiments, Z^{b} is O or NH. In some embodiments, Z^{b}R⁵ taken together is NO₂ or N3. In some embodiments, R⁶ is H, methyl, methoxy, nitro or chloro. In some embodiments, R⁷ is methyl. In some embodiments, R⁸, R⁹, R¹⁰, R¹¹ and R¹² are each independently H or methyl. In some embodiments, R¹⁰ is methyl. In some embodiments, when Y is —CH₂—, X is not an amino acid, peptide, or —P(O)(OH)₂ group.

According to another embodiment, the compound of Formula (I) is a compound having the structure of Formula (III):

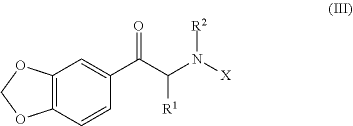

or a pharmaceutically acceptable salt thereof, wherein:
X is independently selected from the group consisting of:
   (a) an amino acid or a peptide;
   (b) —C(O)R³,
   (c) —C(O)OR³,
   (d) —C(O)OCH(R⁴)ORs,
   (e) —CH₂OC(O)R³,
   (f) —P(O)(OH)₂,
   (g) —CH₂OP(O)(OH)₂,
   (h) —C(O)(CH₂)ₙZ^{a}R⁵,

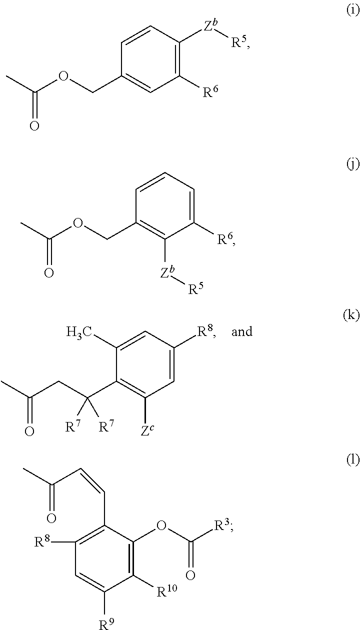

wherein:
n is 3 or 4;
R¹ and R² are each independently —C₁₋₆alkyl or —C₃₋₆cycloalkyl;
R³ is selected from the group consisting of: —C₁₋₆alkyl, —C₃₋₆cycloalkyl, —C₁₋₆haloalkyl, aryl, heteroaryl,

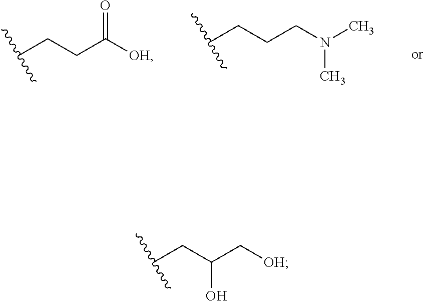

R⁴, R⁷, R⁸, R⁹ and R¹⁰ are each independently H, —C₁₋₆alkyl or —C₃₋₆cycloalkyl;
R⁵ is selected from the group consisting of: —C(O)R³, —C(O)OR³, —P(O)OR¹¹(OR¹²), an amino acid, and a peptide;
Z^{a} and Z^{b} are each independently 0 or NR⁴;
Z^{c} is selected from: OC(O)R³ or OP(O)(OR⁴)₂;
R⁶ is selected from the group consisting of: H, —C₁₋₆alkyl, —C₃₋₆cycloalkyl, alkoxy, amino, nitro, halo, cyano, —OH and CF₃;
R¹¹ and R¹² are each independently H, —C₁₋₆alkyl, —C₁₋₆heteroalkyl, —C₃₋₆cycloalkyl, —C₁₋₆haloalkyl, aryl or heteroaryl, wherein —C₁₋₆alkyl, —C₁₋₆heteroalkyl, —C₃₋₆cycloalkyl, —C₁₋₆haloalkyl, aryl or heteroaryl is unsubstituted or substituted with one or more —C₁₋₆alkyl, —C₁₋₆heteroalkyl, —C₃₋₆cycloalkyl, —C₁₋₆haloalkyl, aryl or heteroaryl.

In some embodiments of the foregoing compounds, R¹ and R² are each independently methyl or ethyl. In some embodiments, R⁴ is H or methyl. In some embodiments, Z^{a}, is NH. In some embodiments, Z^{b} is O or NH. In some embodiments, Z^{b}R⁵ taken together is NO₂ or N3. In some embodiments, R⁶ is H, methyl, methoxy, nitro or chloro. In some embodiments, R⁷ is methyl. In some embodiments, R⁸, R⁹, R¹⁰, R¹¹ and R¹² are each independently H or methyl. In some embodiments, R¹⁰ is methyl. In some embodiments, X is an amino acid. In some embodiments, the compound is selected from the group consisting of compounds 1-402 of Table 1, 2 and 3 below.

According to some embodiments, the compound of Formula (I) is a compound having the structure of Formula (IV):

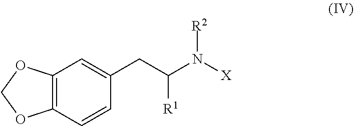

or a pharmaceutically acceptable salt thereof, wherein:
X is independently selected from the group consisting of:
   (a) —C(O)R³,
   (b) —C(O)OR³,
   (c) —C(O)OCH(R⁴)OR⁵,
   (d) —CH₂OC(O)R³,
   (e) —CH₂OP(O)(OH)₂,
   (f) —C(O)(CH₂)ₙZ^{a}R⁵,

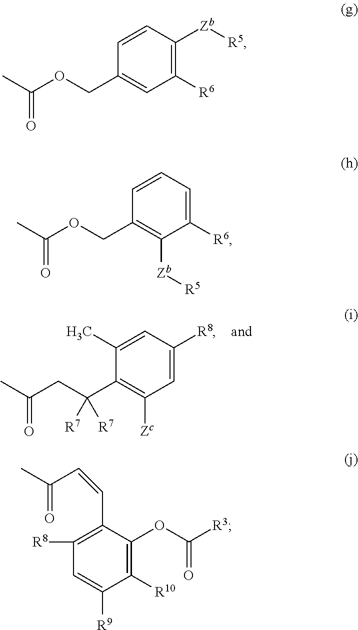

wherein:
n is 3 or 4;
R¹ and R² are each independently —C₁₋₆alkyl or —C₃₋₆cycloalkyl;
R³ is selected from the group consisting of: —C₁₋₆alkyl, —C₃₋₆cycloalkyl, —C1. ₆haloalkyl, aryl, heteroaryl,

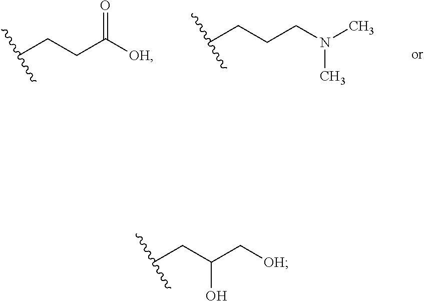

R⁴, R⁷, R⁸, R⁹ and R¹⁰ are each independently H, —C₁₋₆alkyl or —C₃₋₆cycloalkyl;
R⁵, is selected from the group consisting of: —C(O)R³, —C(O)OR³, —P(O)OR¹¹(OR¹²), an amino acid, and a peptide;
Z^{a} and Z^{b} are each independently 0 or NR⁴;
Z^{c} is selected from: OC(O)R³ or OP(O)(OR⁴)₂;
R⁶ is selected from the group consisting of: H, —C₁₋₆alkyl, —C₃₋₆cycloalkyl, alkoxy, amino, nitro, halo, cyano, —OH and CF₃;
R¹¹ and R¹² are each independently H, —C₁₋₆alkyl, —C₁₋₆heteroalkyl, —C₃₋₆cycloalkyl, —C₁₋₆haloalkyl, aryl or heteroaryl, wherein —C₁₋₆alkyl, —C₁₋₆heteroalkyl, —C₃₋₆cycloalkyl, —C₁₋₆haloalkyl, aryl or heteroaryl is unsubstituted or substituted with one or more —C₁₋₆alkyl, —C₁₋₆heteroalkyl, —C₃₋₆cycloalkyl, —C₁₋₆haloalkyl, aryl or heteroaryl.

In some embodiments of the foregoing compounds, R¹ and R² are each independently methyl or ethyl. In some embodiments, R⁴ is H or methyl. In some embodiments, Z^{a}, is NH. In some embodiments, Z^{b} is O or NH. In some embodiments, Z^{b}R⁵ taken together is NO₂ or N3. In some embodiments, R⁶ is H, methyl, methoxy, nitro or chloro. In some embodiments, R⁷ is methyl. In some embodiments, R⁸, R⁹, R¹⁰, R¹¹ and R¹² are each independently H or methyl. In some embodiments, R¹⁰ is methyl.

According to some embodiments, the compound of Formula (I) is a compound having the structure of Formula (V):

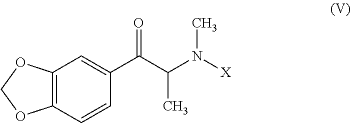

or a pharmaceutically acceptable salt thereof, wherein:
X is independently selected from the group consisting of:
   (a) an amino acid or a peptide;
   (b) —C(O)R³,
   (c) —C(O)OR³,
   (d) —C(O)OCH(R⁴)OR⁵,
   (e) —CH₂OC(O)R³,
   (f) —P(O)(OH)₂,
   (g) —CH₂OP(O)(OH)₂,
   (h) —C(O)(CH₂)ₙZ^{a}R⁵,

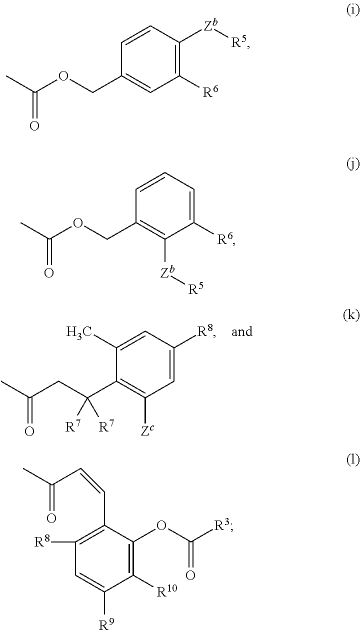

wherein:
n is 3 or 4;
R³ is selected from the group consisting of: —C₁₋₆alkyl, —C₃₋₆cycloalkyl, —C₁₋₆haloalkyl, aryl, heteroaryl,

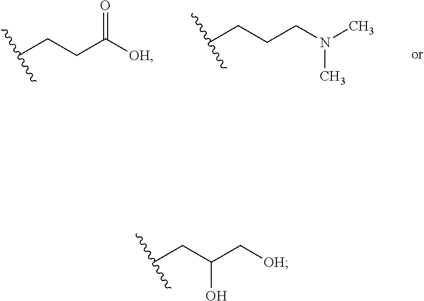

R⁴, R⁷, R⁸, R⁹ and R¹⁰ are each independently H, —C₁₋₆alkyl or —C₃₋₆cycloalkyl;
R⁵ is selected from the group consisting of: —C(O)R³, —C(O)OR³, —P(O)OR¹¹(OR¹²), an amino acid, and a peptide;
Z^{a} and Z^{b} are each independently 0 or NR⁴;
Z^{c} is selected from: OC(O)R³ or OP(O)(OR⁴)₂;
R⁶ is selected from the group consisting of: H, —C₁₋₆alkyl, —C₃₋₆cycloalkyl, alkoxy, amino, nitro, halo, cyano, —OH and CF₃;
R¹¹ and R¹² are each independently H, —C₁₋₆alkyl, —C₁₋₆heteroalkyl, —C₃₋₆cycloalkyl, —C₁₋₆haloalkyl, aryl or heteroaryl, wherein —C₁₋₆alkyl, —C₁₋₆heteroalkyl, —C₃₋₆cycloalkyl, —C₁₋₆haloalkyl, aryl or heteroaryl is unsubstituted or substituted with one or more —C₁₋₆alkyl, —C₁₋₆heteroalkyl, —C₃₋₆cycloalkyl, —C₁₋₆haloalkyl, aryl or heteroaryl.

In some embodiments of the foregoing compounds, R⁴ is H or methyl. In some embodiments, Z^{a}, is NH. In some embodiments, Z^{b} is O or NH. In some embodiments, Z^{b}R⁵ taken together is NO₂ or N3. In some embodiments, R⁶ is H, methyl, methoxy, nitro or chloro. In some embodiments, R⁷ is methyl. In some embodiments, R⁸, R⁹, R¹⁰, R¹¹ and R¹² are each independently H or methyl. In some embodiments, R¹⁰ is methyl. In some embodiments, X is an amino acid.

According to some embodiments, the compound of Formula (I) is a compound having the structure of Formula (VI):

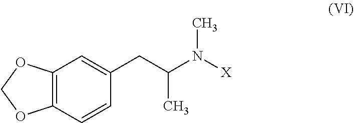

or a pharmaceutically acceptable salt thereof, wherein:
X is independently selected from the group consisting of:
   (a) —C(O)R³,
   (b) —C(O)OR³,
   (c) —C(O)OCH(R⁴)OR⁵,
   (d) —CH₂OC(O)R³,
   (e) —CH₂OP(O)(OH)₂,
   (f) —C(O)(CH₂)ₙZ^{a}R⁵,

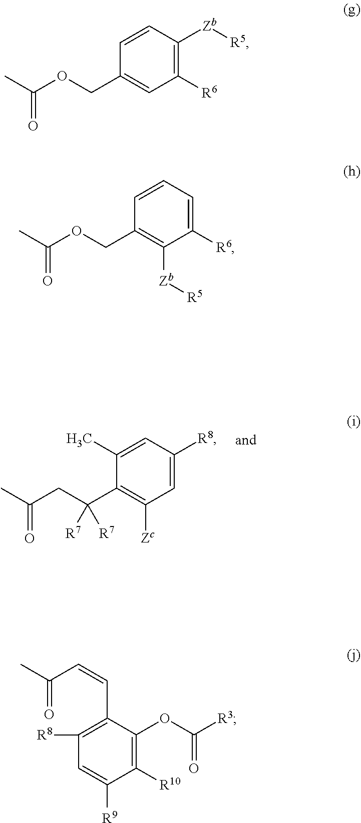

wherein:
n is 3 or 4;
R³ is selected from the group consisting of: —C₁₋₆alkyl, —C₃₋₆cycloalkyl, —C₁₋₆haloalkyl, aryl, heteroaryl,

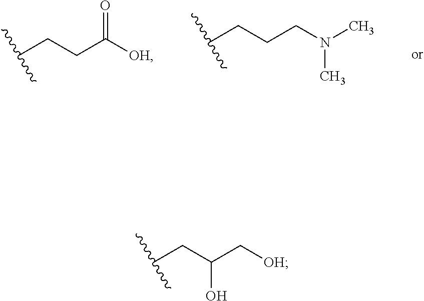

R⁴, R⁷, R⁸, R⁹ and R¹⁰ are each independently H, —C₁₋₆alkyl or —C₃₋₆cycloalkyl;
R⁵, is selected from the group consisting of: —C(O)R³, —C(O)OR³, —P(O)OR¹¹(OR¹²), an amino acid, and a peptide;
Z^{a} and Z^{b} are each independently 0 or NR⁴;
Z^{c} is selected from: OC(O)R³ or OP(O)(OR⁴)₂;
R⁶ is selected from the group consisting of: H, —C₁₋₆alkyl, —C₃₋₆cycloalkyl, alkoxy, amino, nitro, halo, cyano, —OH and CF₃;
R¹¹ and R¹² are each independently H, —C₁₋₆alkyl, —C₁₋₆heteroalkyl, —C₃₋₆cycloalkyl, —C₁₋₆haloalkyl, aryl or heteroaryl, wherein —C₁₋₆alkyl, —C₁₋₆heteroalkyl, —C₃₋₆cycloalkyl, —C₁₋₆haloalkyl, aryl or heteroaryl is unsubstituted or substituted with one or more —C₁₋₆alkyl, —C₁₋₆heteroalkyl, —C₃₋₆cycloalkyl, —C₁₋₆haloalkyl, aryl or heteroaryl.

In some embodiments, R⁴ is H or methyl. In some embodiments, Z^{a}, is NH. In some embodiments, Z^{b} is O or NH. In some embodiments, Z^{b}R⁵ taken together is NO₂ or N3. In some embodiments, R⁶ is H, methyl, methoxy, nitro or chloro. In some embodiments, R⁷ is methyl. In some embodiments, R⁸, R⁹, R¹⁰, R¹¹ and R¹² are each independently H or methyl. In some embodiments, R¹⁰ is methyl. In some embodiments, the compound is selected from the group consisting of compounds 403-511 of Table 4 below.

For some embodiments of the foregoing compounds, the amino acid, dipeptide, tripeptide or polypeptide may comprise one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine.

Without wishing to be bound by theory, prodrugs of cathinones, such as methylone, or of phenethylamines, such as MDMA, are believed to act as a systemic controlled release system of the parent molecule active principal through in vivo bioactivation. Such bioactivation can be accomplished by either the enzymatic or chemical cleavage of the covalently bound promoiety or by a combination of both enzymatic and chemical cleavage of the covalently bound promoiety.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C≡C bond.

The term “haloalkyl” refers to an alkyl group having 1-9 halo groups attached. Examples include —CH₂F, —CHF₂, —CF₃, —CH₂CH₂F, —CHFCH₂F, —CF₂CH₂F, —CF₂CHF₂ and —CF₂CF₃.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalenyl, adamantanyl, indanyl, indenyl, fluorenyl, 1,2,3,4-tetrahydronaphthalenyl and the like. Similarly, “cycloalkenyl” means carbocycles containing no heteroatoms and at least one non-aromatic C═C double bond, and include mono-, bi- and tricyclic partially saturated carbocycles, as well as benzofused cycloalkenes. Examples of cycloalkenyl include cyclohexenyl, indenyl, and the like.

The term “cycloalkyloxy” unless specifically stated otherwise includes a cycloalkyl group connected to the oxy connecting atom.

The term “alkoxy” unless specifically stated otherwise includes an alkyl group connected to the oxy connecting atom.

The term “aryl” unless specifically stated otherwise includes multiple ring systems as well as single ring systems such as, for example, phenyl or naphthyl.

The term “aryloxy” unless specifically stated otherwise includes multiple ring systems as well as single ring systems such as, for example, phenyl or naphthyl, connected through the oxy connecting atom to the connecting site.

The term “C₀-C₆alkyl” includes alkyls containing 6, 5, 4, 3, 2, 1, or no carbon atoms. An alkyl with no carbon atoms is a hydrogen atom substituent when the alkyl is a terminus moiety. An alkyl with no carbon atoms is a direct bond when the alkyl is a bridging moiety.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The heteroatoms replace ring carbon atoms. Thus, for example, a heterocycloCsalkyl is a five membered ring containing from 5 to no carbon atoms. Examples of heteroaryl include, pyridinyl, quinolinyl, isoquinolinyl, pyridazinyl, pyrimidinyl, pyrazinyl, quinoxalinyl, furyl, benzofuryl, dibenzofuryl, thienyl, benzothienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, tetrazolyl.

The term “heteroaryloxy” unless specifically stated otherwise describes a heteroaryl group connected through an oxy connecting atom to the connecting site. Examples of heteroaryl(C₁₋₆)alkyl include, for example, furylmethyl, furylethyl, thienylmethyl, thienylethyl, pyrazolylmethyl, oxazolylmethyl, oxazolylethyl, isoxazolylmethyl, thiazolylmethyl, thiazolylethyl, imidazolylmethyl, imidazolylethyl, benzimidazolylmethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridinylmethyl, pyridinylethyl, pyridazinylmethyl, pyrimidinylmethyl, pyrazinylmethyl, quinolinylmethyl, isoquinolinylmethyl and quinoxalinylmethyl. Examples of heterocycloC₃₋₇alkyl include, for example, azetidinyl, pyrrolidinyl, piperidinyl, perhydroazepinyl, piperazinyl, morpholinyl, tetrahydrofuranyl, imidazolinyl, pyrolidin-2-one, piperidin-2-one, and thiomorpholinyl.

The term “N-heterocycloC₄₋₇alkyl” describes nonaryl heterocyclic compounds having 3-6 carbon atoms and one nitrogen atom forming the ring. Examples include azetidinyl, pyrrolidinyl, piperidinyl, and perhydroazepinyl. Examples of aryl(C₁₋₆)alkyl include, for example, phenyl(C₁₋₆)alkyl, and naphthyl(C₁₋₆)alkyl. Examples of heterocycloC₃₋₆alkylcarbonyl(C₁₋₆)alkyl include, for example, azetidinyl carbonyl(C₁₋₆)alkyl, pyrrolidinyl carbonyl(C₁₋₆)alkyl, piperidinyl carbonyl(C₁₋₆)alkyl, piperazinyl carbonyl(C₁₋₆)alkyl, morpholinyl carbonyl(C₁₋₆)alkyl, and thiomorpholinyl carbonyl(C₁₋₆)alkyl.

The term “amine” unless specifically stated otherwise includes primary, secondary and tertiary amines.

Unless otherwise stated, the term “carbamoyl” includes —NHC(O)OC₁₋C₄alkyl, and —OC(O)NHC₁—C₄alkyl.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, the substitution can be made at any of the groups. For example, substituted aryl(C₁₋₆)alkyl includes substitution on the aryl group as well as substitution on the alkyl group.

The term “oxide” of heteroaryl groups is used in the ordinary well-known chemical sense and include, for example, N-oxides of nitrogen heteroatoms.

The term “polymorphic forms” refers to different crystalline forms of the same compound, drug substance or active ingredient; these can include solvation or hydration products (also known as pseudo-polymorphs) and amorphous forms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included.

During the course of the synthetic procedures used to prepare such compounds, or in using racemization or epimerization procedures known to those skilled in the art, the products of such procedures can be mixtures of stereoisomers.

In one aspect, the present invention provides a concise route for the preparation of methylone enantiomers. The processes of the present invention can yield substantially pure methylone enantiomers. For (S)-methylone, by “substantially pure” is meant that compound (S)-methylone is at least substantially separated from the environment in which it was formed or detected. Substantial purity can include compositions containing at least about 80.0%, or at least about 85.0%, or at least about 90.0%, or at least about 95.0%, or at least about 97.0%, or at least about 98.0%, or at least about 99.0%, or at least about 99.2%, or at least about 99.4%, or at least about 99.6%, or at least about 99.8%, or at least about 99.9%, or even about 100% by weight of the compound. For (R)-methylone, by “substantially pure” is meant that compound (R)-methylone is at least substantially separated from the environment in which it was formed or detected. Substantial purity can include compositions containing at least about 80.0%, or at least about 85.0%, or at least about 90.0%, or at least about 95.0%, or at least about 97.0%, or at least about 98.0%, or at least about 99.0%, or at least about 99.2%, or at least about 99.4%, or at least about 99.6%, or at least about 99.8%, or at least about 99.9%, or even about 100% by weight of the compound.

Embodiments of the invention also include compositions comprising (S)-methylone. Preferably, these compositions are pharmaceutical compositions comprising (S)-methylone and at least one pharmaceutically acceptable excipient. In some embodiments, the compositions and pharmaceutical compositions may be prepared with substantially pure (S)-methylone. In some embodiments, the compositions and pharmaceutical compositions have an enantiomeric excess (EE) of at least 90% EE, preferably at least 95% EE, more preferably at least 98% EE, and even more preferably at least 99% EE and most preferably about 100% EE. The compositions and pharmaceutical compositions may also be prepared as mixture of the enantiomeric forms of the compounds (e.g., as a racemic mixture or as a mixture with a ratio of 60:40, 70:30, 80:20 or 90:10 of (S)-methylone relative to (R)-methylone).

Embodiments of the invention also include compositions comprising (R)-methylone. Preferably, these compositions are pharmaceutical compositions comprising (R)-methylone and at least one pharmaceutically acceptable excipient. In some embodiments, the compositions and pharmaceutical compositions may be prepared with substantially pure (R)-methylone. In some embodiments, the compositions and pharmaceutical compositions have an enantiomeric excess (EE) of at least 90% EE, preferably at least 95% EE, more preferably at least 98% EE, and even more preferably at least 99% EE and most preferably about 100% EE. The compositions and pharmaceutical compositions may also be prepared as mixture of the enantiomeric forms of the compounds (e.g., as a racemic mixture or as a mixture with a ratio of 60:40, 70:30, 80:20 or 90:10 of (R)-methylone relative to (S)-methylone).

In another aspect, provided herein are pharmaceutical compositions comprising a compound described herein or a pharmaceutically acceptable salt thereof; and a pharmaceutically acceptable carrier. In some embodiments, the pharmaceutical composition comprises a salt of a compound described herein.

A “pharmaceutical composition” is a formulation containing compounds in a form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used herein includes both one and more than one such excipient.

The term “pharmaceutically acceptable salts” refers to salts prepared from pharmaceutically acceptable non-toxic bases or acids or co-crystal formers. The crystalline form can exist as salt, solvate, hydrate, or clathrate. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases or co-crystals from which salts or co-crystals can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When a compound of the present invention is basic, its corresponding salt or co-crystals can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

According to some embodiments, pharmaceutical compositions comprising a compound represented by Formula (I) (or pharmaceutically acceptable salts or co-crystals thereof) as an active ingredient, and a pharmaceutically acceptable carrier and optionally other therapeutic ingredients or adjuvants may be prepared.

According to another embodiment, the pharmaceutical compositions of this invention may include a pharmaceutically acceptable carrier/excipients, a compound or a pharmaceutically acceptable salt/co-crystal of Formula (I) and the corresponding parent psychoactive agent of the compound of Formula (I).

Dosage levels from about 0.0001 mg/kg to about 100 mg/kg of body weight per day may be useful in the treatment of conditions such as: post-traumatic stress disorder (PTSD), anxiety disorder, attention deficit hyperactivity disorder (ADHD), obsessive compulsive disorder (OCD), fibromyalgia, depression, acute stress disorder (ASD), cluster headache, a condition associated with cancer, diminished drive, burn-out, bore-out, migraine, Parkinson's disease, pulmonary hypertension, schizophrenia, an eating disorder, nausea, or vomiting. Dosage levels from about 0.0001 mg/kg to about 100 mg/kg of body weight per day may be useful in the treatment of conditions such as: mood disorders, anxiety disorders, personality disorders, fibromyalgia, suicidal ideation, substance use disorders (SUD), eating disorders, Borderline Personality Disorder (BPD) and other personality disorders, obsessive-compulsive disorder (OCD), palliative care/end-of-life anxiety, existential distress, chronic pain syndromes, body dysmorphia, phobias, social anxiety in autistic adults, and sleep regulation, by the administration of an effective amount of the phenethylamine or cathinone precursor.

The amount of active ingredient that may be combined with the carrier materials to produce a single dosage form will vary depending upon the treated target and the particular mode of administration. For example, a formulation intended for the oral administration to humans may conveniently contain from about 0.5 mg to about 5 g of active agent, formulated with an appropriate and acceptable amount of “GRAS” materials which may vary from about 5 to about 95 percent of the total composition. Unit dosage forms will generally contain between about 0.001 mg to about 5000 mg of the active ingredient, typically 0.001 mg, 0.005 mg, 0.025 mg, 0.1 mg, 0.5 mg, 2.5 mg, 5.0 mg, 10 mg, 30 mg, 60 mg, 100 mg, 300 mg, 600 mg, 1000 mg, 3000 mg, 5000 mg or any dose in-between.

Pharmaceutical compositions suitable for use as described herein include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. It is understood, however, that the specific dose level for any particular patient will depend upon a variety of factors including the age, body weight, general health, sex, diet, time of administration, route of administration, rate of excretion, drug combination and the severity of the particular disease undergoing therapy.

The composition, shape and type of dosage forms provided herein will typically vary depending on their use. For example, a dosage form used in the acute treatment of a disease may contain large amounts of one or more of the active ingredients including Formula (I) it comprises than a dosage form used in the chronic treatment of the same disease. Similarly, a parenteral dosage form may contain smaller amounts of one or more of the active ingredients including Formula (I) it comprises than an oral dosage form used to treat the same disease. These and other ways in which specific dosage forms provided herein will vary from one another will be readily apparent to those skilled in the art. See, e.g., Remington's Pharmaceutical Sciences, 20^{th} ed., Mack Publishing, Easton, Pa (2000). In practice, the compounds represented by Formula (I), or pharmaceutically acceptable salts/co-crystals thereof, of this disclosure can be combined as the active ingredient in intimate admixture with a pharmaceutical excipients, carrier, or diluents according to conventional pharmaceutical compounding techniques. The carrier may take a wide variety of forms depending on the form of preparation desired for administration, e.g., oral, mucosal (e.g., nasal, sublingual, vaginal, inhalational, cystic, rectal, ocular, buccal or aural), parenteral (including intravenous, intradermal, subcutaneous, bolus injection, intramuscular or intraarterial) or topical (e.g., transdermal, transcutaneous, eye drops or other ophthalmic preparations). Thus, the pharmaceutical compositions of the present disclosure can be presented as discrete units suitable for oral administration such as capsules (coated or non-coated with polymers as sustained release or enteric coated or modified for target delivery), sachets or tablets (coated or uncoated or bilayers or sustained release or delayed release including micro-encapsulation) or tablets containing spray dried intermediates each containing a predetermined amount of the active ingredient. Further, the compositions can be presented as a powder, as granules, as a coated sustained release particle, as a solution, as a suspension in an aqueous liquid, as a non-aqueous liquid, as an oil-in-water emulsion or as a water-in-oil liquid emulsion, liposomes, nanosuspension. In addition to the common dosage forms set out above, the compound represented by Formula (I), or pharmaceutically acceptable salts or co-crystals thereof, may also be administered by controlled or modified release formulation and/or delivery devices. The compositions may be prepared by any of the methods of pharmacy. In general, such methods include a step of bringing into association the active ingredient with the excipients or carriers that constitutes one or more necessary ingredients. In general, the compositions are prepared by uniformly and intimately admixing the active ingredient with liquid carriers/excipients or finely divided solid carriers/excipients or both. The product can then be conveniently shaped into the desired presentation.

In some embodiments, an amount or a dose of an active ingredient provided herein ranges from 5-250 mg. In some embodiments, an amount or a dose of an active ingredient provided herein is less than 50 mg. In some embodiments, an amount or a dose of an active ingredient provided herein ranges from 5-50 mg. In some embodiments, an amount or a dose of an active ingredient provided herein is less than 25 mg. In some embodiments an amount or a dose of an active ingredient provided herein ranges from 5-25 mg. In some embodiments, an amount or a dose of an active ingredient provided herein ranges from 50-350 mg. In some embodiments, an amount or a dose of an active ingredient provided herein ranges from 50-500 mg. In some embodiments, an amount or a dose of an active ingredient provided herein ranges from 5-1,000 mg.

In some embodiments, an amount or a dose of an active ingredient provided herein may be in the range of about 1 mg to about 100 mg. For example, the amount or the dose administered of an active ingredient may be about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, the amount or the dose administered of an active ingredient provided herein is between about 0.1 mg to about 100 mg, about 1 mg to about 50 mg, or about 5 mg to about 30 mg. In some embodiments, the amount or the dose administered of an active ingredient provided herein is about 1 mg, about 10 mg, or about 25 mg. In some embodiments, the amount or the dose administered of an active ingredient provided herein is in the range of about 0.001 mg to about 1 g. In some embodiments, the amount or the dose administered of an active ingredient provided herein is in the rage of about 100 mg to about 250 mg. In some embodiments, the amount or the dose administered of an active ingredient provided herein is about 25 mg.

In some embodiments, the active ingredient provided herein is administered daily. In some embodiments, the active ingredient is administered twice a day. In some embodiments, the active ingredient is administered three times a day. In some embodiments, the active ingredient is administered every other day. In some embodiments, the active ingredient is administered every third day. In some embodiments, the active ingredient is administered every fourth day. In some embodiments, the active ingredient is administered every fifth day. In some embodiments, the active ingredient is administered weekly. In some embodiments, the active ingredient is administered every other week. In some embodiments, the active ingredient is administered every third week. In some embodiments, the active ingredient is administered monthly.

Thus, the pharmaceutical compositions of the present disclosure may include a pharmaceutically acceptable carrier/excipients and a compound or a pharmaceutically acceptable salt/co-crystal of Formula (I). The compounds of Formula (I), or pharmaceutically acceptable salts/co-crystals thereof, can also be included in pharmaceutical compositions in combination with one or more other therapeutically active compounds.

The pharmaceutical carrier employed can be, for example, to form oral solid preparations such as powders, capsules and tablets include fillers such as talc, calcium carbonate, microcrystalline cellulose, kaolin, mannitol, silicic acid, sorbitol, starch, and mixture thereof. Binder such as Kollidon. Disintegrants such as croscarmellose sodium, crospovidone, sodium starch glycolate, pre-gelatinized starch, gums and other starches and mixtures thereof. Lubricants such as calcium stearate, magnesium stearate, syloid silica gel, mineral oil, glycerine, sorbitol, mannitol, polyethylene glycol, stearic acid, sodium lauryl sulphate, talc, hydrogenated vegetable oil (e.g., peanut oil, sesame oil, corn oil or soybean oil), ethyl oleate agar or other lipid formulation lubricants and mixtures thereof. Because of their ease of administration, tablets and capsules are the preferred oral dosage units whereby solid pharmaceutical carriers are employed. Each of the solid oral dosage units can be further coated with specialized polymers that can delay release or sustained release the contents of the dosage units. Formula (I) can be administered by delayed release or sustained release means or by delivery devices that are well known to those of ordinary skill in the art. Non-limiting examples of delayed release or sustained release include those described in U.S. Pat. Nos. 3,845,770; 3,916,899; 3,536,809; 5,059,595. Such dosage forms can be used to provide slow or controlled release of one or more ingredients using for example, polymers such as hydroxylpropylmethyl cellulose usually in a matrix form such as gel, permeable membranes, micro-emulsions, osmotic systems, liposomes, microspheres or combinations thereof. Controlled release formulation can be used to protect the dosage units from exposure to the gastric environment; delay release of active ingredient to the lower gastrointestinal tract such as the colon; or slow the release of the active ingredient such that blood levels of the drug can be lowered and affect the occurrence of side effects.

Examples of gaseous carriers include carbon dioxide and nitrogen.

In preparing the oral liquid compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions.

A tablet containing the composition of the present disclosure may be prepared by compression or molding, optionally with one or more accessory ingredients or adjuvants.

Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.001 mg to about 5000 mg of the active ingredient and each cachet or capsule preferably containing from about 0.001 mg to about 5000 mg of the active ingredient.

Pharmaceutical compositions of the present disclosure suitable for parenteral administration (including intravenous, intramuscular, subcutaneous, ocular, and intraarterial) may be prepared as solutions or suspensions of the active compounds in injectable ingredients. Parenteral dosage forms are preferably sterile or capable of being sterilized prior to administration to a patient. Non-limiting examples of suitable vehicles include Water for Injection USP; Dextrose Injection; Sodium Chloride Injection and lactated Ringer's Injection. A suitable surfactant can be included such as, for example, polysorbate 80. Dispersions can also be prepared in glycerol, liquid polyethylene glycols, ethyl alcohol, polypropylene glycol and mixtures thereof in non-aqueous vehicles such as oils (e.g., corn oil, sesame oil, isopropyl myristate). An antioxidant to help stabilize the formulation such as ascorbic acid or ascorbyl palmitate. Further, a preservative can be included to prevent the detrimental growth of microorganisms.

Furthermore, the compositions can be in the form of sterile powders for the extemporaneous preparation of such sterile injectable solutions or dispersions. In all cases, the final injectable form must be sterile, non-irritating with addition of tonicity agents and must be effectively fluid for easy syringeability. The pharmaceutical compositions must be stable under the conditions of manufacture and storage; thus, preferably should be preserved against the contaminating action of microorganisms such as bacteria and fungi such as benzalkonium chloride, chlorobutanol, methyl paraben, propyl paraben, edetate disodium, sorbic acid or other agents known to those skilled in the art. Pharmaceutical compositions of the present disclosure can be in a form suitable for topical applied locally to the skin and its adnexa or to a variety of mucous membranes such as, for example, an aerosol, patch, cream, ointment, lotion, dusting powder, emulsions or the like. The routes that can be used include nasal, sublingual, vaginal, rectal, ocular, buccal or aural. Further, the compositions can be in a form suitable for use in transdermal or intradermal micro-needle devices. These formulations may be prepared, utilizing a compound represented by Formula (I) of the present disclosure, or pharmaceutically acceptable salts thereof, via conventional processing methods. As an example, a lotion, cream or ointment is prepared by mixing hydrophilic material and water, together with about 5 wt % to about 30 wt % of the compound, to produce a cream, lotion or ointment having a desired consistency. Examples of typical excipients include water, acetone, ethanol, ethylene glycol, propylene glycol, isopropyl myristate, mineral oil and mixtures thereof. Moisturizers such as occlusive, humectant, emollients can also be added to the pharmaceutical compositions and dosage forms if desired. pH of a pharmaceutical composition or dosage form may also be adjusted to improve delivery of Formula (I). Dosage forms suitable for treating mucosal tissues within the oral cavity can be formulated as mouthwashes or as oral gel.

Pharmaceutical compositions of the present disclosure can be in a form suitable for rectal administration wherein the carrier is a solid or liquid or spray. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, binders, surface-active agents, thickeners, lubricants, preservatives (including antioxidants) and the like. Furthermore, other adjuvants can be included to render the formulation isotonic with the blood of the intended recipient. Compositions containing a compound described by Formula (I), or pharmaceutically acceptable salts thereof, may also be prepared in powder or liquid concentrate form. Addition of preservatives such as antioxidants are widely acceptable in pharmaceutical arts as a means of simulating long-term storage in order to determine characteristics such as shelf life or stability of formulations over time (See, e.g., Jens T. Carstensen, Drug stability: Principles & Practice. 2^{nd} Ed., Marcel Dekker, NY, NY. 1995, pp 379-80).

All diseases, conditions, and disorders listed herein are defined as described in the Diagnostic and Statistical Manual of Mental Disorders (DSM-5), published by the American Psychiatric Association, or in International Classification of Diseases (ICD), published by the World Health Organization.

The term “ICH Q3A” refers to the guidelines and standards described in the International Council for Harmonisation of Technical Requirements for Pharmaceuticals for Human Use (2006) Impurities in new drug substances: Q3A(R2), available at www.ema.europa.eu/en/ich-q3a-r2-impurities-new-drug-substances-scientific-guideline. The term “ICH Q3B” refers to the guidelines and standards described in the International Council for Harmonisation of Technical Requirements for Pharmaceuticals for Human Use (2006) Impurities in new drug products: Q3B(R2), available www.ema.europa.eu/en/ich-q3b-r2-impurities-new-drug-products-scientific-guideline. The term “ICH Q3C” refers to the guidelines and standards described in the International Council for Harmonisation of Technical Requirements for Pharmaceuticals for Human Use (2021) Impurities: guideline for residual solvents: ICH Q3C(R8). The term “ICH Q3D” refers to the guidelines and standards described in the International Council for Harmonisation of Technical Requirements for Pharmaceuticals for Human Use (2019) Guideline for elemental impurities: ICH Q3D(RI).

As used herein, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

As used herein, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

In one embodiment, a variety of other therapeutic agents may find use for administration with the compositions and methods provided herein.

In another aspect, provided herein are methods of treating and/or preventing a disease or condition, such as a neuropsychiatric illness, and/or ameliorating a symptom thereof in a subject in need thereof comprises administering to the subject an effective amount of a compound or composition provided herein. In some embodiments, the disease or condition is selected from post-traumatic stress disorder (PTSD), anxiety disorder, attention deficit hyperactivity disorder (ADHD), obsessive compulsive disorder (OCD), fibromyalgia, depression, acute stress disorder (ASD), cluster headache, a condition associated with cancer, diminished drive, burn-out, bore-out, migraine, Parkinson's disease, pulmonary hypertension, schizophrenia, an eating disorder, nausea, or vomiting. In some embodiments, the disease or condition is selected from mood disorders, anxiety disorders, personality disorders, fibromyalgia, suicidal ideation, substance use disorders (SUD), eating disorders, Borderline Personality Disorder (BPD) and other personality disorders, obsessive-compulsive disorder (OCD), palliative care/end-of-life anxiety, existential distress, chronic pain syndromes, body dysmorphia, phobias, social anxiety in autistic adults, and sleep regulation, by the administration of an effective amount of the phenethylamine or cathinone precursor. In some embodiments, the disease or condition is PTSD. In some embodiments, the disease or condition is an anxiety disorder. In some embodiments, the disease or condition is depression.

In some embodiments, the neuropsychiatric illness is a Depressive Disorder. In some embodiments, the Depressive Disorder is selected from the group consisting of Disruptive Mood Dysregulation Disorder, Major Depressive Disorder, Single and Recurrent Episodes, Persistent Depressive Disorder (Dysthymia), Premenstrual Dysphoric Disorder, Substance/Medication-Induced Depressive Disorder, Depressive Disorder Due to Another Medical Condition, Other Specified Depressive Disorder, Unspecified Depressive Disorder, and combinations thereof. In some embodiments, the neuropsychiatric illness is post-traumatic stress disorder (PTSD). In some embodiments, the neuropsychiatric illness is acute stress disorder. In some embodiments, the neuropsychiatric illness is Fibromyalgia. In some embodiments, the neuropsychiatric illness is a mood disorder. In some embodiments, the neuropsychiatric illness is an anxiety disorder. In some embodiments, the Anxiety Disorder is selected from the group consisting of Generalized anxiety disorder, Panic disorder, Panic attack, Phobic anxiety disorders, Illness Anxiety Disorder, dissociative, stress-related, somatoform other nonpsychotic mental disorders, acute stress reaction, transient adjustment reaction, neurasthenia, psychophysiologic disorders, Obsessive-compulsive disorder, Reaction to severe stress and adjustment disorders, Separation Anxiety Disorder, episodic paroxysmal anxiety, Selective Mutism, Specific Phobia, Social Anxiety Disorder (Social Phobia), Agoraphobia, Substance/Medication-Induced Anxiety Disorder, Anxiety Disorder Due to Another Medical Condition, Anxiety in pregnancy and childbirth, Anxiety in pregnancy antepartum (before childbirth), Anxiety postpartum, Animal type phobia, Arachnophobia, Other animal type phobia, Natural environment type phobia, Fear of thunderstorms, Fear of blood, Fear of injections and transfusions, Fear of other medical care, Fear of injury, Situational type phobia, Claustrophobia, Acrophobia, Other Unspecified Anxiety Disorder, Body Dysmorphic Disorder Hoarding Disorder Trichotillomania (Hair-Pulling Disorder) Excoriation (Skin-Picking), and combinations thereof. In some embodiments, the neuropsychiatric illness is an eating disorder. In some embodiments, the neuropsychiatric illness is a Personality Disorder (PD). In some embodiments, the Personality Disorder is selected from the group consisting of Borderline Personality Disorder (BPD), Avoidant Personality Disorder (AvPD), Antisocial Personality Disorder (AsPD), Schizotypal Personality Disorder, Other Anxiety and Panic producing Disorders, Specific personality disorders, Impulse disorders, Gender identity disorders, Paraphilias, Other sexual disorders, Other disorders of adult personality and behavior, Unspecified disorder of adult personality and behavior, Personality and behavioral disorders due to known physiological conditions. In some embodiments, the subject with the PD also has a Depressive Disorder. In some embodiments, the neuropsychiatric illness is a Somatic Symptom Disorders. In some embodiments, the Somatic Symptom Disorder is selected from the group consisting of Illness Anxiety Disorder, Conversion Disorder (Functional Neurological Symptom Disorder), Psychological Factors Affecting Other Medical Conditions, Factitious Disorder, Other Specified Somatic Symptom and Related Disorder, Unspecified Somatic Symptom and Related Disorder, and combinations thereof. In some embodiments, the subject is suicidal. In some embodiments, the neuropsychiatric illness is treatment-resistant.

The compounds provided herein may be used for various therapeutic purposes. In one embodiment, the compounds are administered to a subject to treat a neuropsychiatric illness. A “subject” for the purposes of the compositions and methods provided herein includes humans and other animals, preferably mammals and most preferably humans. Thus, the compounds provided herein have both human therapy and veterinary applications. In another embodiment the subject is a mammal, and in yet another embodiment the subject is human. By “condition”, “disease”, or “illness” herein are meant a disorder that may be ameliorated by the administration of compounds provided herein and pharmaceutical compositions thereof.

Methods and compositions described herein can be used for prophylaxis, as well as amelioration of signs and/or symptoms of a condition, such as a neuropsychiatric illness. The terms “treating” and “treatment” used to refer to treatment of a condition in a subject include: preventing, inhibiting or ameliorating the condition in the subject, as well as reducing or ameliorating a sign or symptom of the condition. Treatment goals may incorporate endpoints such as improvement in DSM-5 severity scales, to measure if resilience and quality of life are enhanced, with engagement of positive cognitive valence systems, and corresponding reduction in negative valence.

It is to be understood by one of skill in the art that the methods of treatment and/or prevention comprising administering a compound provided herein for the treatment and/or prevention of one or more indications as described herein also include: the use of a compound provided herein in the manufacture of a medicament for the treatment and/or prevention of one or more indications as described herein; and the use of a compound provided herein for the treatment and/or prevention of one or more indications as described herein.

Pharmaceutical compositions are contemplated for the compounds and methods provided herein. Formulations of the compositions and methods provided herein are prepared for storage by mixing said compound having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g., Zn-protein complexes); and/or non-ionic surfactants or polyethylene glycol (PEG). In another embodiment, the pharmaceutical compositions provided herein are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

Pharmaceutically acceptable excipients for formulations of compounds provided herein include, but are not limited to: diluents, e.g., microcrystalline cellulose, starch, mannitol, calcium hydrogen phosphate anhydrous or co-mixtures of silicon dioxide, calcium carbonate, microcrystalline cellulose and talc; disintegrants, e.g., sodium starch glycolate or croscarmellose sodium; binders, e.g., povidone, co-povidone or hydroxyl propyl cellulose; lubricants, e.g., magnesium stearate or sodium stearyl fumurate; glidants, e.g., colloidal silicon dioxide; and film coats, e.g., Opadry II white or PVA based brown Opadry II.

The compounds provided herein can be purified. A compound herein can be least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

The term “impurity” refers to any component of a drug product that is not the drug substance or an excipient in or a carrier of the drug product. An “impurity profile is a description of the identified and unidentified impurities present in a drug product. An “identified impurity” is an impurity for which a structural characterization has been achieved, while an “unidentified impurity” is an impurity for which a structural characterization has not been achieved and that is defined solely by qualitative analytical properties (e.g., chromatographic retention time). A “potential impurity” is an impurity that theoretically can arise during manufacture or storage; it may or may not actually appear in the drug substance or active ingredient.

The term “degradation product” refers to an impurity resulting from a chemical change in the drug substance or active ingredient brought about during manufacture and/or storage of the drug product or active ingredient by the effect of, for example, light, temperature, pH, water, or by reaction with an excipient and/or carrier and/or the immediate container closure system. A “degradation profile” is a description of the degradation products observed in the drug substance, drug product or active ingredient. An “identified degradation product” is a degradation product for which a structural characterization has been achieved, while an “unidentified degradation product” is a degradation product for which a structural characterization has not been achieved and that is defined solely by qualitative analytical properties (e.g., chromatographic retention time).

In some embodiments, the compound of Formula (I), or pharmaceutically acceptable salts thereof, is of a purity of at least 99% by HPLC, such as a purity of at least 99.5% by HPLC. In some embodiments, the compound of formula I, or a pharmaceutically acceptable salt thereof, is of a purity of at least 99.9% by HPLC, such as a purity of at least 99.95% by HPLC.

In some embodiments, the compound of Formula (I), or pharmaceutically acceptable salts thereof, produces two or fewer impurity peaks by HPLC. In some embodiments, where the compound of formula I, or a pharmaceutically acceptable salt thereof, produces no impurity peak by HPLC that is greater than 0.2%. In some embodiments, no impurity peak by HPLC that is greater than 0.15%. In some embodiments, no impurity peak by HPLC that is greater than 0.1%.

In another aspect, the present disclosure is directed to pharmaceutical compositions of the compounds described herein, such as methylone, including pharmaceutically acceptable salts of methylone and/or stereoisomers of methylone, and/or isotopologues and isotopomers of methylone as well as polymorphs and other solid forms of any of the foregoing. In one embodiment, the pharmaceutical compositions of methylone are high-purity pharmaceutical compositions of methylone. In one embodiment, the pharmaceutical compositions of methylone are room temperature stable pharmaceutical compositions of methylone. In one embodiment, the pharmaceutical compositions of methylone are not mutagenic and lack mutagenic impurities. In one embodiment, the pharmaceutical compositions of methylone are suitable for use in humans. In one embodiment, the pharmaceutical compositions of methylone are commercial scale pharmaceutical compositions of methylone.

In some embodiments, the pharmaceutical compositions of methylone described herein conform to the microbiological guidelines and standards as described in USP. General Chapter <1111>Microbiological Examination of Nonsterile Products: Acceptance Criteria for Pharmaceutical Preparations and Substances for Pharmaceutical Use. In: The United States Pharmacopeia 43—National Formulary 38. Rockville, MD: United States Pharmacopeial Convention; 2020 and/or the European Pharmacopoeia Commission. General Chapter 5.1.4. Microbiological Quality of Pharmaceutical Preparations. European Pharmacopoeia, 10th Edition, 2020.

Provided herein are high-purity pharmaceutical compositions comprising methylone, including pharmaceutically acceptable salts of methylone (e.g., methylone HCl) and/or stereoisomers of methylone e.g., (S)-methylone), and/or isotopologues and isotopomers of methylone, as well as polymorphs and other solid forms of any of the foregoing; and a pharmaceutically acceptable carrier. In some embodiments, the high-purity pharmaceutical compositions of methylone are room temperature stable. In some embodiments, the high-purity pharmaceutical compositions of methylone are not mutagenic and lack mutagenic impurities. In some embodiments, the high-purity pharmaceutical compositions of methylone are suitable for use in humans. In some embodiments, the high-purity pharmaceutical compositions of methylone are commercial scale pharmaceutical compositions of methylone. In some embodiments, the high-purity pharmaceutical compositions of methylone conform to the qualification thresholds set forth in the ICH Q3A and ICH Q3B guidelines. In some embodiments, the high-purity pharmaceutical compositions of methylone have levels of residual solvents that conform to the standards set forth in the ICH Q3C guidelines. In some embodiments, the high-purity pharmaceutical compositions of methylone have levels of elemental impurities that conform to the standards set forth in the ICH Q3D guidelines.

In some embodiments, the high-purity pharmaceutical compositions comprising methylone or a stereoisomer thereof (e.g., (S)-methylone), or a pharmaceutically acceptable salt thereof (e.g., methylone HCl) is at least 99.5% pure, at least 99.6% pure, at least 99.7% pure, at least 99.8% pure, at least 99.9% pure, at least 99.91% pure, at least 99.92% pure, at least 99.93% pure, at least 99.94% pure, at least 99.95% pure, at least 99.96% pure, at least 99.97% pure, at least 99.98% pure, at least 99.99% pure, or greater than 99.99% pure.

In some embodiments, the high-purity pharmaceutical compositions comprise racemic methylone that is at least 99.5% pure, at least 99.6% pure, at least 99.7% pure, at least 99.8% pure, at least 99.9% pure, at least 99.91% pure, at least 99.92% pure, at least 99.93% pure, at least 99.94% pure, at least 99.95% pure, at least 99.96% pure, at least 99.97% pure, at least 99.98% pure, at least 99.99% pure, or greater than 99.99% pure. In some embodiments, the high-purity pharmaceutical compositions comprise (S)-methylone that is at least 99.5% pure, at least 99.6% pure, at least 99.7% pure, at least 99.8% pure, at least 99.9% pure, at least 99.91% pure, at least 99.92% pure, at least 99.93% pure, at least 99.94% pure, at least 99.95% pure, at least 99.96% pure, at least 99.97% pure, at least 99.98% pure, at least 99.99% pure, or greater than 99.99% pure. In some embodiments, the high-purity pharmaceutical compositions comprise (R)-methylone that is at least 99.5% pure, at least 99.6% pure, at least 99.7% pure, at least 99.8% pure, at least 99.9% pure, at least 99.91% pure, at least 99.92% pure, at least 99.93% pure, at least 99.94% pure, at least 99.95% pure, at least 99.96% pure, at least 99.97% pure, at least 99.98% pure, at least 99.99% pure, or greater than 99.99% pure. In some embodiments, the high-purity pharmaceutical compositions comprise methylone HCl that is at least 99.5% pure, at least 99.6% pure, at least 99.7% pure, at least 99.8% pure, at least 99.9% pure, at least 99.91% pure, at least 99.92% pure, at least 99.93% pure, at least 99.94% pure, at least 99.95% pure, at least 99.96% pure, at least 99.97% pure, at least 99.98% pure, at least 99.99% pure, or greater than 99.99% pure.

In some embodiments, the high-purity pharmaceutical compositions comprising methylone or a stereoisomer thereof (e.g., (S)-methylone), or a pharmaceutically acceptable salt thereof (e.g., methylone HCl) has two or fewer impurity peaks by HPLC. In some embodiments, the high-purity pharmaceutical compositions comprising methylone or a stereoisomer thereof (e.g., (S)-methylone), or a pharmaceutically acceptable salt thereof (e.g., methylone HCl) has one impurity peak by HPLC. In some embodiments, the high-purity pharmaceutical compositions comprising methylone or a stereoisomer thereof (e.g., (S)-methylone), or a pharmaceutically acceptable salt thereof (e.g., methylone HCl) has no impurity peak by HPLC that is greater than 0.04%, greater than 0.03%, greater than 0.02%, greater than 0.01%, or greater than 0.005%. In some embodiments, the high-purity pharmaceutical compositions comprising methylone or a stereoisomer thereof (e.g., (S)-methylone), or a pharmaceutically acceptable salt thereof (e.g., methylone HCl) has no impurities detectable by HPLC.

In some embodiments, the high-purity pharmaceutical compositions have one or more impurities selected from 2,3-Methylone, 2-bromo-3′,4′-(methylenedioxy)propiophenone (MDPBP) and 3,4-methylenedioxypropiophenone (MDP) detectable by HPLC. In some embodiments, the high-purity pharmaceutical compositions have no 2,3-Methylone detectable by HPLC. In some embodiments, the high-purity pharmaceutical compositions have no 2-bromo-3′,4′-(methylenedioxy)propiophenone (MDPBP) detectable by HPLC. In some embodiments, the high-purity pharmaceutical compositions have no 3,4-methylenedioxypropiophenone (MDP) detectable by HPLC. In some embodiments, the high-purity pharmaceutical compositions have no 2,3-Methylone detectable by HPLC.

In another aspect, provided herein are pharmaceutical compositions that are room temperature stable and comprise methylone, including pharmaceutically acceptable salts of methylone (e.g., methylone HCl) and/or stereoisomers of methylone e.g., (S)-methylone), and/or isotopologues and isotopomers of methylone, as well as polymorphs and other solid forms of any of the foregoing; and a pharmaceutically acceptable carrier. In some embodiments, the room temperature stable pharmaceutical compositions are high-purity pharmaceutical compositions. In some embodiments, the room temperature stable pharmaceutical compositions are formulated in an oral dosage form, e.g., as a tablet or capsule. In some embodiments, the room temperature stable pharmaceutical compositions of methylone are not mutagenic and lack mutagenic impurities. In some embodiments, the room temperature stable pharmaceutical compositions of methylone are suitable for use in humans. In some embodiments, the room temperature stable pharmaceutical compositions of methylone are commercial scale pharmaceutical compositions of methylone. In some embodiments, the room temperature stable pharmaceutical compositions of methylone conform to the qualification thresholds set forth in the ICH Q3A and ICH Q3B guidelines. In some embodiments, the room temperature stable pharmaceutical compositions of methylone have levels of residual solvents that conform to the standards set forth in the ICH Q3C guidelines. In some embodiments, the room temperature stable pharmaceutical compositions of methylone have levels of elemental impurities that conform to the standards set forth in the ICH Q3D guidelines.

A room temperature stable pharmaceutical composition of methylone means that after a specified interval of time (e.g., one month, three months, six months, nine months, twelve months, eighteen months, twenty-four months, or thirty-six months) the pharmaceutical composition when assayed by HPLC, the amount of methylone is 90-110%, preferably between 97-103%, of the initial amount of methylone and the impurities conform to the qualification thresholds set forth in the ICH Q3A and ICH Q3B guidelines and total impurities is not more than 5%. In some embodiments, the total impurities is not more than 4.5%, not more than 4%, not more than 3.5%, not more than 3%, not more than 2.5%, not more than 2%, not more than 1.5%, not more than 1%, or not more than 0.5%.

In some embodiments, the room temperature stable pharmaceutical compositions comprise racemic methylone that is room temperature stable for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions comprise (S)-methylone that is room temperature stable for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, room temperature stable pharmaceutical compositions comprise (R)-methylone that is room temperature stable for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions comprise methylone HCl that is room temperature stable for at least one month, at least three months, at least six months, at least seven months, at least eight months, at least nine months, at least ten months, at least eleven months, at least one year, at least fifteen months, at least eighteen months, at least twenty-one months, at least two years, or at least three years.

In some embodiments, the room temperature stable pharmaceutical compositions are stable when stored or kept at a temperature of between 15° C. and 30° C. for at least one month, at least three months, at least six months, at least seven months, at least eight months, at least nine months, at least ten months, at least eleven months, at least one year, at least fifteen months, at least eighteen months, at least twenty-one months, at least two years, or at least three years.. In some embodiments, the room temperature stable pharmaceutical compositions are stable when stored or kept at a temperature of between 25° C. and 30° C. for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions are stable when stored or kept at a temperature of 20±2C for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions are stable when stored or kept at a temperature of 22±2C for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions are stable when stored or kept at a temperature of 24±2C for at least one month, at least three months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions are stable when stored or kept at a temperature of 25±2C for at least one month, at least three months, at least six months, at least seven months, at least eight months, at least nine months, at least ten months, at least eleven months, at least one year, at least fifteen months, at least eighteen months, at least twenty-one months, at least two years, or at least three years.

In some embodiments, the room temperature stable pharmaceutical compositions are stable when stored or kept at a relative humidity (RH) of at least 60% for at least one month, at least two months, at least three months, at least four months, at least five months, at least six months, at least seven months, at least eight months, at least nine months, at least ten months, at least eleven months, at least one year, at least fifteen months, at least eighteen months, at least twenty-one months, at least two years, or at least three years. In some embodiments, the room temperature stable pharmaceutical compositions are stable when stored or kept at a relative humidity of at least 75% for at least one month, at least two months, at least three months, at least four months, at least five months, at least six months, at least seven months, at least eight months, at least nine months, at least ten months, at least eleven months, at least one year, at least fifteen months, at least eighteen months, at least twenty-one months, at least two years, or at least three years.

In some embodiments, the room temperature stable pharmaceutical compositions are stable when stored or kept at a temperature of 40° C. and a relative humidity (RH) of at 75% for at least one month, at least two months, at least three months, at least four months, at least five months, or at least six months.

Provided herein are non-mutagenic pharmaceutical compositions comprising methylone, including pharmaceutically acceptable salts of methylone (e.g., methylone HCl) and/or stereoisomers of methylone e.g., (S)-methylone), and/or isotopologues and isotopomers of methylone, as well as polymorphs and other solid forms of any of the foregoing; and a pharmaceutically acceptable carrier. In some embodiments, the non-mutagenic pharmaceutical compositions of methylone are room temperature stable. In some embodiments, the non-mutagenic pharmaceutical compositions of methylone are high-purity pharmaceutical compositions of methylone. In some embodiments, the non-mutagenic pharmaceutical compositions of methylone are suitable for use in humans. In some embodiments, the non-mutagenic pharmaceutical compositions of methylone are commercial scale pharmaceutical compositions of methylone. In some embodiments, pharmaceutical compositions of methylone are determined to be non-mutagenic using an in vitro Ames test. In some embodiments, the non-mutagenic pharmaceutical compositions of methylone conform to the qualification thresholds set forth in the ICH Q3A and ICH Q3B guidelines. In some embodiments, the non-mutagenic pharmaceutical compositions of methylone have levels of residual solvents that conform to the standards set forth in the ICH Q3C guidelines. In some embodiments, the non-mutagenic pharmaceutical compositions of methylone have levels of elemental impurities that conform to the standards set forth in the ICH Q3D guidelines.

In some embodiments, the pharmaceutical compositions of methylone satisfy at lease one, at least two, at least three, at least four, at least five, at least six, at least seven, at least eight, at least nine, or at least ten of the specifications set forth in TABLE 1. In some embodiments, the pharmaceutical compositions of methylone satisfy all of the specifications set forth in TABLE 1.

TABLE 1

| Test | Method | Specification | | |
|---|---|---|---|---|
| Appearance | Visual | White to off-white, blueish-gray crystalline solid | | |
| Identity | Ph. Eur/USP<197>; | Conforms to reference spectrum | | |
| | FTIR | | | |
| Loss on Drying | Ph. Eur/USP<731> | Record Result | | |
| (LOD) | | | | |
| Water Content | Ph. Eur/USP<921>; | NMT 0.5% | | |
| | KF | | | |
| Chloride Content | Ph. Eur/USP<221>; | 14.5 ± 2% w/w | | |
| | titration | | | |
| Assay | HPLC | 97.0-103.0% | | |
| Related | HPLC | 2,3-Methylone: NMT 0.15% | | |
| Substances | | MDP: NMT 0.15% | | |
| | | MDPBP: NMT 0.15% | | |
| | | Any Unspecified Impurity: NMT 0.10% | | |
| | | Total Impurities: NMT 1.0% | | |
| Residual | Ph. Eur/USP <467>; | Conforms to ICH Q3C | | |
| Solvents | GC | Benzene (Class 1) | 2 | ppm |
| | | Toluene (Class 2) | 890 | ppm |
| | | Methylisobutyl Ketone (Class 2) | 4500 | ppm |
| | | Isopropanol (Class 3) | 5000 | ppm |
| | | Methanol (Class 2) | 3000 | ppm |

| | | |
|---|---|---|
| Elemental | Ph. Eur/USP <233> | Conforms to ICH Q3D |
| Impurities | | Class 1 |

| Arsenic (As) | ≤1.5 | ppm |
|---|---|---|
| Cadmium (Cd) | ≤0.5 | ppm |
| Lead (Pb) | ≤0.5 | ppm |
| Mercury (Hg) | ≤4 | ppm |
| Class 2A | | |
| Cobalt (Co) | ≤5 | ppm |
| Molybdenum (Mo) | ≤18 | ppm |
| Selenium (Se) | ≤17 | ppm |
| Vanadium (V) | ≤12 | ppm |
| Process Related Elementals | | |
| Copper (Cu) | ≤130 | ppm |

| | | |
|---|---|---|
| Microbial | Ph. Eur./USP | TAMC ≤ 1000 CFU/G |
| Limits | | TYMC ≤ 100 CFU/G |
| | | Absence: Sa, Pa. Ec, Sal, Cs, BTGN, Ca |

NMT = not more than

In some embodiments, the pharmaceutical compositions of methylone are formulated in an oral dosage form as a capsule and satisfy at lease one, at least two, at least three, at least four, or at least five of the specifications set forth in TABLE 2. In some embodiments, the pharmaceutical compositions of methylone satisfy all of the specifications set forth in TABLE 2.

TABLE 2

| Test | Method | Reference | Specification |
|---|---|---|---|
| Appearance | Visual | Internal Procedure | White opaque capsules containing |
| | | | white to off-white, bluish-grey, |
| | | | crystalline powder |
| Identification by | HPLC | Internal Procedure | The retention time of the principal |
| HPLC Retention | | | peak in the sample solution is within |
| Time | | | 98.0-102.0% of the average |
| | | | retention time of methylone in |
| | | | bracketing injections of working |
| | | | standard solution. |
| Related | HPLC | Internal Procedure | Any Individual Impurity NMT 0.2% |
| Substances | | | Single Largest Unspecified Impurity |
| | | | NMT 0.2% |
| | | | Total Impurities NMT 1.0% |
| Fill Weight | Weight | Internal Procedure | ±5% 50 mg drug substance weight |
| | | | adjusted for purity, water content, |
| | | | and chloride content |
| Capsule Closure | Visual & Physical | Internal Procedure | Passes Test |
| TAMC, EP 5.1.4/ | EP 2.6.12 & 2.6.13 | Ph. Eur./USP | ≤10³ CFU/G |
| USP<1111> | USP<61><62> | | |
| TYMC, EP 5.1.4/ | EP 2.6.12 & 2.6.13 | Ph. Eur./USP | ≤10² CFU/G |
| USP <1111> | USP<61><62> | | |
| Absence of E. coli | EP 2.6.12 & 2.6.13 | Ph. Eur./USP | Absence |
| in 1 g, EP 5.1.4/ | USP<61><62> | | |
| USP <1111> | | | |

In some embodiments, the pharmaceutical compositions of methylone are formulated in an oral dosage form as a capsule that after a specified interval of time (e.g., one month, three months, six months, nine months, twelve months, eighteen months, twenty-four months, or thirty-six months) satisfy at lease one, at least two, at least three, at least four, or at least five of the specifications set forth in TABLE 3. In some embodiments, the pharmaceutical compositions of methylone satisfy all of the specifications set forth in TABLE 3.

TABLE 3

| Test | Method | Reference | Proposed Specification |
|---|---|---|---|
| Appearance | Visual | Internal Procedure | White Opaque capsules containing |
| | | | white to off-white, bluish-grey, |
| | | | crystalline powder |
| Proper | Capsule is Tightly | Internal Procedure | Tightly Closed |
| Closure | Closed | | |
| Assay | HPLC | Internal Procedure | 90.0-110.0% |
| Related | HPLC | Internal Procedure | Any Individual Impurity NMT |
| Substances | | | 0.2% |
| | | | Single Largest Unspecified |
| | | | Impurity NMT 0.2% |
| | | | Total Impurities NMT 1.0% |
| Moisture | Karl Fisher | Ph. Eur./USP | NMT 5% |
| TAMC, EP 5.1.4/ | EP 2.6.12 & 2.6.13 | Ph. Eur./USP | ≤10³ CFU/G |
| USP <1111>** | USP<61><62> | | |
| TYMC, EP 5.1.4/ | EP 2.6.12 & 2.6.13 | Ph. Eur./USP | ≤10² CFU/G |
| USP <1111>** | USP<61><62> | | |
| Absence of E. coli in | EP 2.6.12 & 2.6.13 | Ph. Eur./USP | Absence |
| 1 g, EP 5.1.4/ | USP<61><62> | | |
| USP <1111>** | | | |

The compounds provided herein may also be entrapped in microcapsules prepared by methods including, but not limited to, coacervation techniques, interfacial polymerization (e.g., using hydroxymethylcellulose or gelatin-microcapsules, or poly-(methylmethacylate) microcapsules), colloidal drug delivery systems (e.g., liposomes, albumin microspheres, microemulsions, nano-particles and nano-capsules), and macroemulsions. Sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides, copolymers of L-glutamic acid and gamma ethyl-L-glutamate, non-degradable ethylene-vinyl acetate, degradable lactic acid-glycolic acid copolymers (which are injectable microspheres composed of lactic acid-glycolic acid copolymer and leuprolide acetate), and poly-D-(−)-3-hydroxybutyric acid) which is a micro sphere-b ased delivery system composed of the desired bioactive molecule incorporated into a matrix of poly-DL-lactide-co-glycolide (PLG).

Administration of the pharmaceutical composition comprising the compounds provided herein, for example in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. As is known in the art, the pharmaceutical composition may be formulated accordingly depending upon the manner of

### INTRODUCTION

In some embodiments, the pharmaceutical formulation is an oral dosage form. In some embodiments, the pharmaceutical formulation is a parenteral dosage form. In some embodiments, the pharmaceutical composition comprises a tablet. In some embodiments, the pharmaceutical composition comprises a capsule. In some embodiments, the pharmaceutical composition comprises a dry powder. In some embodiments, the pharmaceutical composition comprises a solution. In some embodiments, more than one dosage form is administered to the subject at substantially the same time. In some embodiments, the subject may be administered the entire therapeutic dose in one tablet or capsule. In some embodiments, the therapeutic dose may be split among multiple tablets or capsules.

As used herein, the singular forms “a,” “an,” and “the” include plural references unless the context clearly dictates otherwise. For example, the term “a molecule” can also include a plurality of molecules.

The terms “about” or “approximately”, which are used interchangeably herein, means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Furthermore, the term “about” as used herein when referring to a measurable value such as a dose, time, temperature, and the like, is meant to encompass variations of ±20%, ±10%, ±5%, ±1%, ±0.5%, or even ±0.1% of the specified amount.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless the context indicates otherwise, it is specifically intended that the various features described herein can be used in any combination.

Any patent, patent application publication, or scientific publication, cited herein, is incorporated by reference herein in its entirety.

The following examples are presented to illustrate preferred embodiments more fully. They should in no way be construed, however, as limiting the broad scope of the invention.

### EXAMPLES

### Example 1

### Methods of Synthesis

The compounds of Formula (I) of the present invention can be prepared according to the proposed synthetic routes outlined in Schemes 1-17 below starting from the parent molecule II. Methylone IIa (Y═CO, R¹═CH₃, R²═CH₃), ethylone IIb (Y═CO, R¹ CH₃, R²═CH₂CH₃), butylone IIc (Y═CO, R¹═CH₂CH₃, R²═CH₃) and MDMA lid (Y═CH₂, R¹'² CH₃, R²═CH₃) can be prepared using procedures such as the one described in WO9639133A1 (IIa); Heather E. et al. Drug Test. Analysis, 2017, 9, 426 (IIa); Maheux C. R. et al. Drug Test. Analysis, 2016, 8, 847 (IIb); Maheux C. R. et al. Drug Test. Analysis, 2012, 4, 17 (IIc) and Milhazes N. et al. Anal. Chem. Act. 2007, 596, 231 (lId).

The amino acid derived prodrugs of Formula 1b and Id may be prepared by coupling the requisite amine II with appropriate amino acids as presented in Scheme 1 below where R¹¹ and R¹² are each independently selected from the side chain residue of the naturally occurring amino acid. To conjugate an amino acid with II, the one amino group is preferably protected with a protecting group (Pg) before the amino acid is reacted with II. Agents and methods for protecting amino groups in a reactant are known in the art. Examples of protecting groups that may be used to protect the amino groups include, but are not limited to, fluorenylmethoxycarbonyl (Fmoc), t-butylcarbonate (Boc), trifluoroacetate (TFA), acetate (Ac) and benzyloxycarbonyl (CBZ). Preferably, the carboxylic acid group in the N-protected amino acid is activated by an acid activating agent (sometimes also called coupling reagent) to help the reaction of the N-protected amino acid with II. Examples of acid activating agents (coupling reagents) well known in the art include, but are not limited to, dicyclohexylcarbodiimide (DCC), 1-ethyl-3-(3′-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU). The use of appropriate acyl halide or anhydride as an activated acylating group in the N-protected amino acid is also contemplated. After coupling with any standard coupling procedure to afford the intermediate protected prodrug 1a, deprotection can occur with standard reagent known in the art to afford the desired prodrugs Ib. This amino acid prodrug can be further derivatized to a dipeptide by repeating the coupling procedure to afford the prodrug Id, after deprotection of the newly added amino group of Ic.

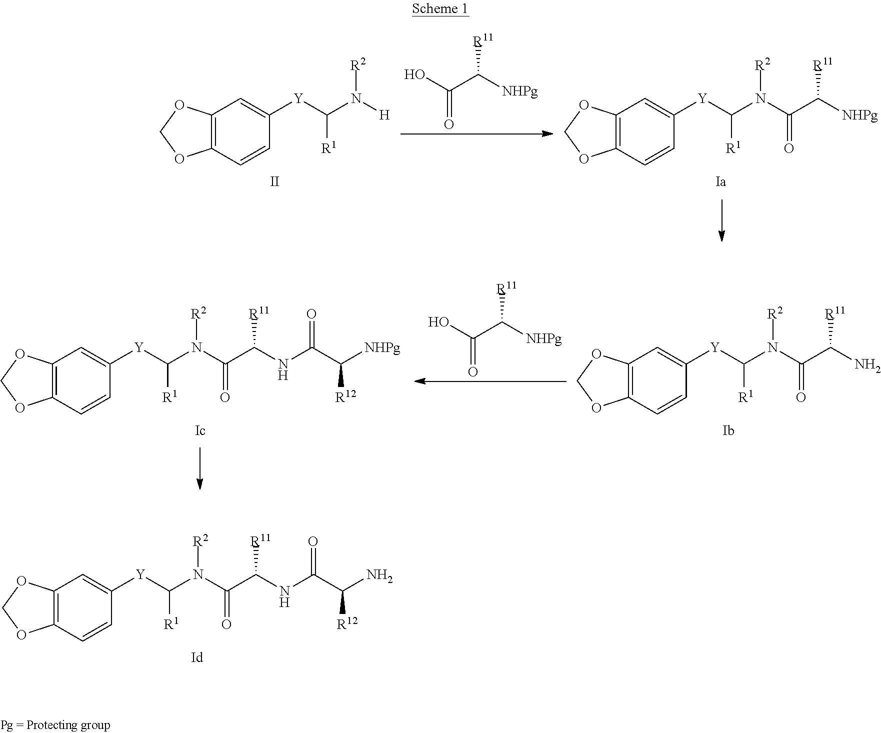

Alternatively, the peptide derived prodrug of Formula Id may be prepared by coupling the requisite amine II and an appropriate dipeptide as presented in Scheme 2 below. Such a conjugation may be accomplished under the conditions previously described for the intermediate 1a (Scheme 1). The requisite dipeptide is provided by the coupling of two amino acids, each independently selected from the naturally occurring L amino acids using standard peptide coupling protocols known in the art.

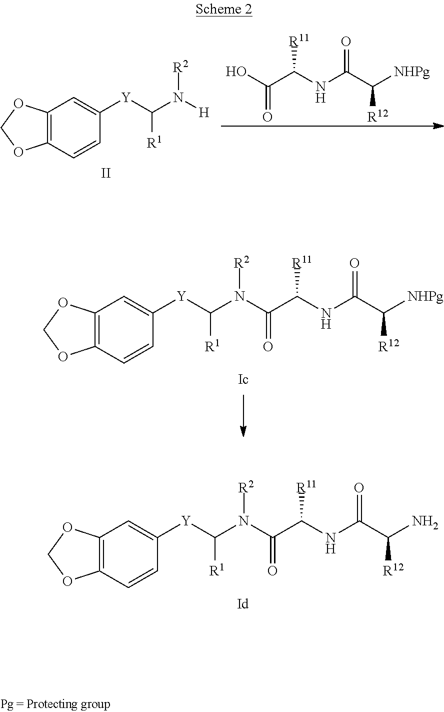

The amide prodrug of Formula e may be prepared by coupling the requisite amine II with an appropriate acylating agent as presented in Scheme 3 below. Acylation of the amino group of II may be accomplished by reaction with an acid chloride (Z═Cl) or anhydride (Z═—OC(O)R³ or —OC(O)t-Butyl) in the presence of a base such as diisopropylethylamine (DIPEA), triethylamine, 4-methylmorpholine, NaHCO₃, K₂CO₃ or 2,6-lutidine in an appropriate solvent such as methylene chloride, THF, DMF, acetonitrile, or toluene. The coupling reaction may also be performed with a carboxylic acid (Z═OH) in the presence of a coupling reagent, such as N,N-dicyclohexylcarbodiimide (DCC), N-ethyl-N′-(3-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU) or other similar reagents well known to one skilled in the art.

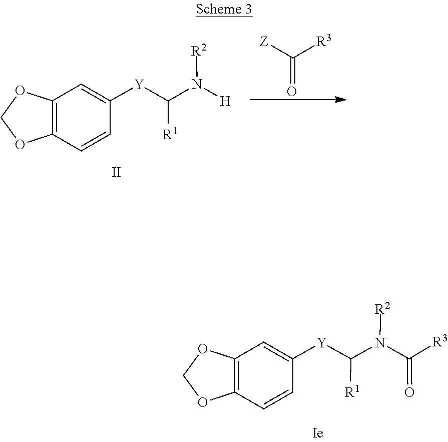

The carbamate prodrug of Formula If may be prepared by coupling the requisite amine II with an appropriate chloroformate as presented in Scheme 4 below. The coupling reaction is performed in the presence of a base such as diisopropylethylamine (DIPEA), triethylamine, NaOH, NaHCO₃, K₂CO₃ or pyridine in an appropriate solvent such as methylene chloride, THF, ethyl acetate, acetonitrile, 1,4-dioxanne or water. Alternatively, the carbamate If can be prepared by the sequential addition of triphosgene to the amine II in presence of a base such as diisopropylethylamine (DIPEA) in a solvent such as methylene chloride, followed by the addition of an alkoxide such as NaOR³.

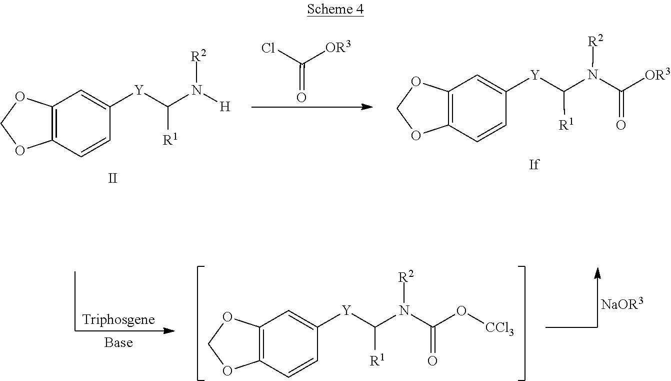

The acyloxyalkoxycarbonyl prodrug of Formula Ig (Scheme 5) may be prepared by sequentially coupling the requisite amine II with 1-chloroethyl chloroformate in the presence of a base, such as triethylamine or diisopropylethylamine in a solvent, such as methylene chloride, followed by the addition of a selected carboxylate. Such a carboxylate may be generated by reacting the corresponding carboxylic acid R³CO₂H with a base such as triethylamine or cesium carbonate in a solvent such as DMF or acetonitrile. Alternatively, the acyloxyalkoxycarbonyl prodrug of Formula Ig could be directly accessed by coupling the requisite amine II with an electrophilic acylating agent such as 1-(((4-nitrophenoxy)carbonyl)oxy)ethyl carboxylate in the presence of a base, such as triethylamine or diisopropylethylamine in a solvent, such as methylene chloride.

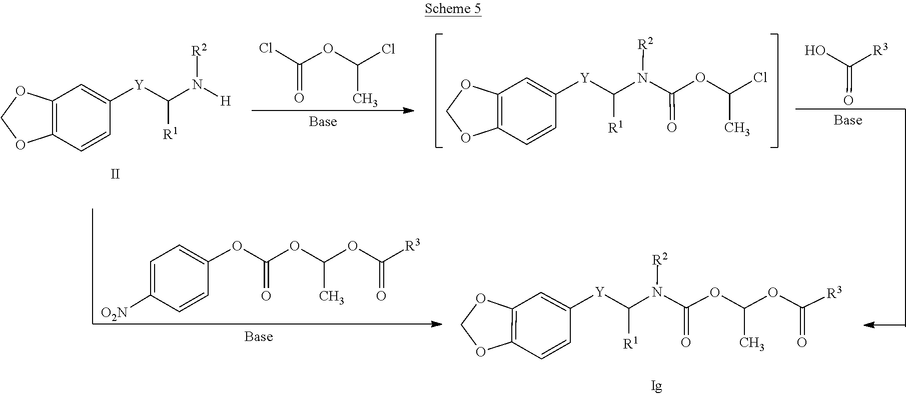

The acyloxymethyl prodrug of Formula Ih may be prepared by coupling the requisite amine II with an appropriate chloromethyl ester in the presence of a basic agent such as triethylamine in a solvent such as acetonitrile (Scheme 6). The chloromethyl ester R³C(O)OCH₂C1 can be prepared according to the procedures described in US20150274670A1 and US 20070155729A1 where the acyl chloride of formula R³COC1 would be reacted with paraformaldehyde.

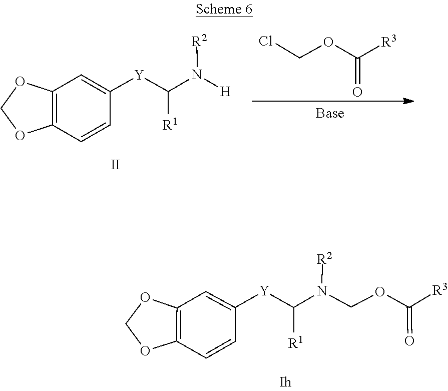

The phosphoramide prodrug of Formula II may be prepared according to the procedure described in WO 2020/008064. As depicted in the Scheme 7 below, PC15 is added to the requisite amine II in the presence of a basic agent such pyridine and in a solvent such as methylene chloride. A mixture of water/DMSO in then added to hydrolyse the dichlorophosphoramide solution to afford the phosphoramide prodrug of Formula II.

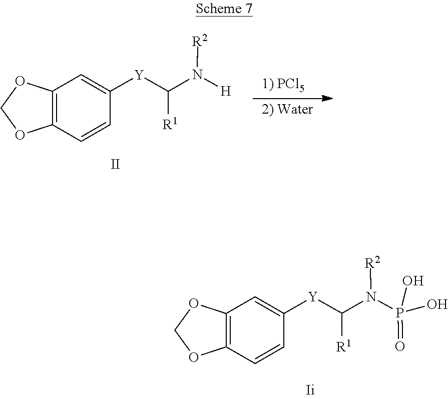

The phosphoryloxymethyl of Formula Ik may be prepared in a two-step sequence from the requisite amine II as presented in Scheme 8 below. Following the procedure found in WO 2020/008064, a solution of amine II in a solvent such as acetonitrile can be treated with a basic agent such K₂CO₃, Nal and di-tert-butyl chloromethylphosphate at a controlled temperature of 50° C. to afford the protected phosphonate Ij. Hydrolysis of this intermediate under aqueous acidic conditions would provide the phosphoryloxymethyl prodrug Ik.

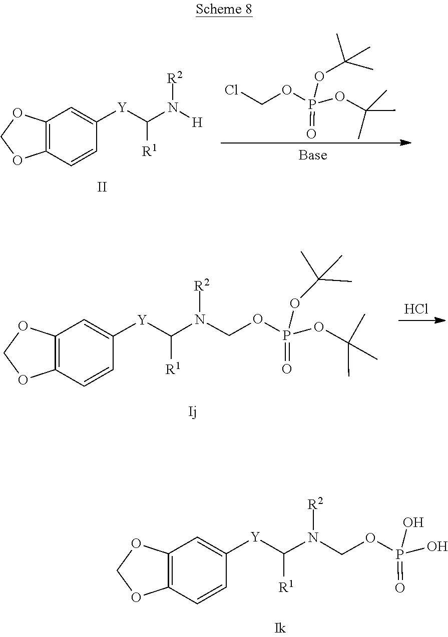

The phosphoryloxyalkoxycarbonyl prodrug of Formula Im (Scheme 9) may be prepared according to the procedure describe by Safadi M. et aL. Pharm Res, 1993, 10(9), 1350, by sequentially coupling the requisite amine II with a chloroalkyl chloroformate in the presence of a base, such as triethylamine or diisopropylethylamine in a solvent, such as methylene chloride, followed by the addition of a suitably protected phosphate such as dibenzyl phosphate (R¹¹ and R¹²=benzyl). Such a phosphate may be generated by reacting the corresponding phosphonic acid with a base such as silver carbonate in a solvent such as DMF or acetonitrile. When R¹¹ and R¹² are benzyl, the dihydrogen phosphate Im may be obtained by deprotecting the phosphate intermediate IL using a catalytic amount of Pd/C under H₂ atmosphere in a solvent such as ethyl acetate.

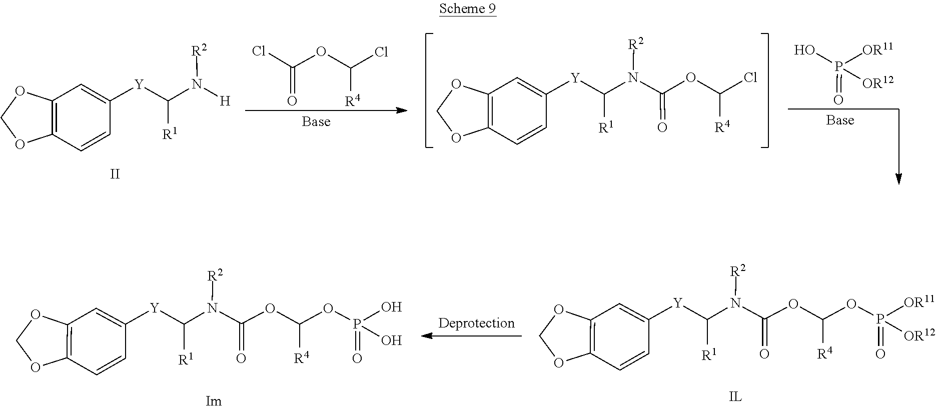

The amide prodrug of Formula Ip may be prepared by coupling the requisite amine II with the carboxylic acid 1o as presented in Scheme 10 below. When Z^{a} is 0, NH or NCH₃, those carboxylic acids may be obtained from a commercial source where a wide diversity of R³ groups such as alkyl, cycloalkyl, aryl, heteroaryl and amino acids could be found. The coupling reaction may be performed in the presence of a coupling reagent, such as N,N-dicyclohexylcarbodiimide (DCC), N-ethyl-N′-(3-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU) or other similar reagents well known to one skilled in the art. In the event that the carboxylic acid would not be commercially available, Io could be prepared by acylation of the amino group (Z^{a}═NR⁴) or of the hydroxy group (Za═O) of In by reaction with a carboxylic acid (Z═OH) in the presence of a coupling agent as described above. Alternatively, the amine In may also be reacted with an acid chloride (Z═Cl) or with an anhydride (Z═—OC(O)R³ or —OC(O)t-Butyl) in the presence of a base such as diisopropylethylamine (DIPEA), triethylamine, 4-methylmorpholine, NaHCO₃, K₂CO₃ or 2,6-lutidine in an appropriate solvent such as methylene chloride, THF, DMF, acetonitrile, or toluene.

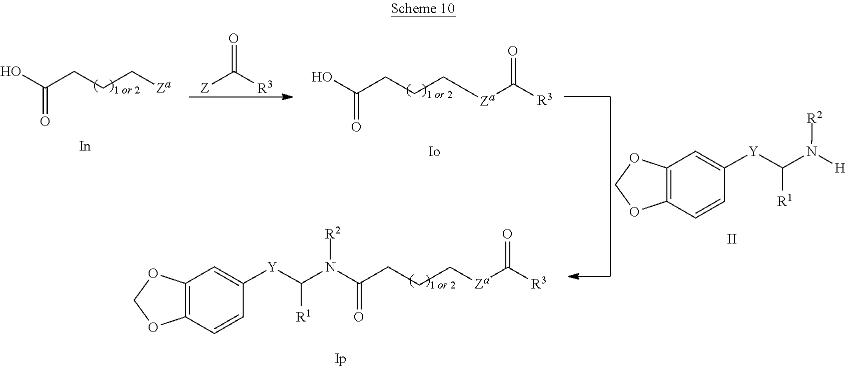

The carbamate prodrug of Formula It may be prepared by coupling the requisite amine II with a benzylic alcohol Is as presented in Scheme 11 below. When Z^{b} is 0, NH or NCH₃, benzylic alcohols may be obtained from a commercial source where a wide diversity of R³ group such as alkyl, cycloalkyl, aryl, heteroaryl and amino acids could be found. The coupling reaction may be performed as described in US 2017/0145044 A1 by the sequential reaction of the benzylic alcohol with a reagent such as carbonyl diimidazole in a solvent such as dichloromethane, followed by the addition of the amine 1l. In the event that the benzylic alcohol would not be commercially available, Is could be prepared following a two-step sequence where the requisite commercially available phenol (Z^{b}═O) or aniline (Z^{b}═NR⁴) Iq would be acylated in an analogous way as described above for Io preparation (Scheme 10), followed by the reduction of the benzaldehyde Ir with a reagent such as sodium borohydride in a solvent such as dichloromethane in the presence of an alcohol such as isopropanol.

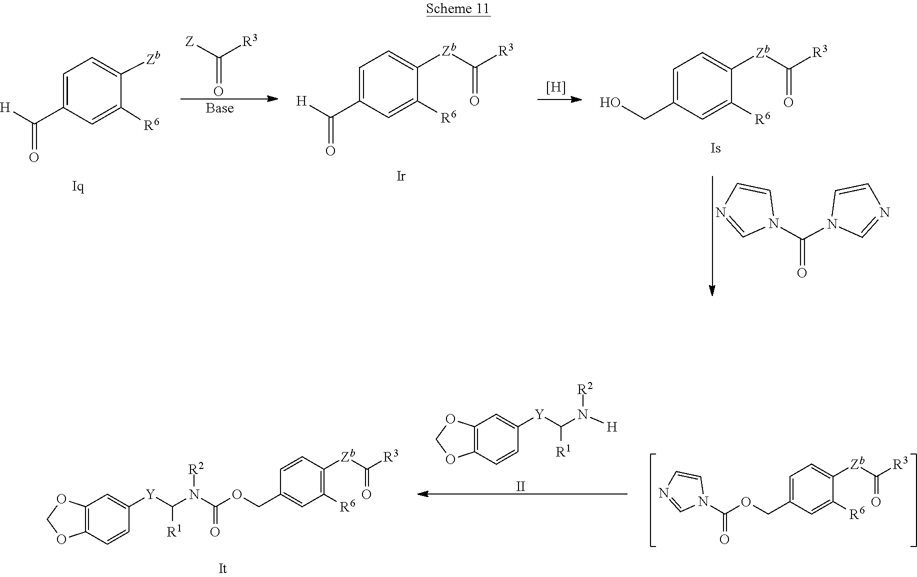

The carbamate prodrug of Formula Ix may be prepared by coupling the requisite amine II with a benzylic alcohol Iw as presented in Scheme 12 below and following an analogous assemblage sequence as described above in Scheme 11. When Z^{b} is O, NH or NCH₃, benzylic alcohols may be obtained from a commercial source where a wide diversity of R³ group such as alkyl, cycloalkyl, aryl, heteroaryl and amino acids could be found. The coupling reaction may be performed by the sequential reaction of the benzylic alcohol with a reagent such as carbonyl diimidazole in a solvent such as dichloromethane, followed by the addition of the amine II. In the event that the benzylic alcohol would not be commercially available, Iw could be prepared following a two-step sequence where the requisite commercially available phenol (Z^{b}═O) or aniline (Z^{b}═NR⁴) lu would be acylated in an analogous way as described above for Io preparation (Scheme 10), followed by the reduction of the benzaldehyde Iv with a reagent such as sodium borohydride in a of solvent such as dichloromethane in the presence of an alcohol such as isopropanol.

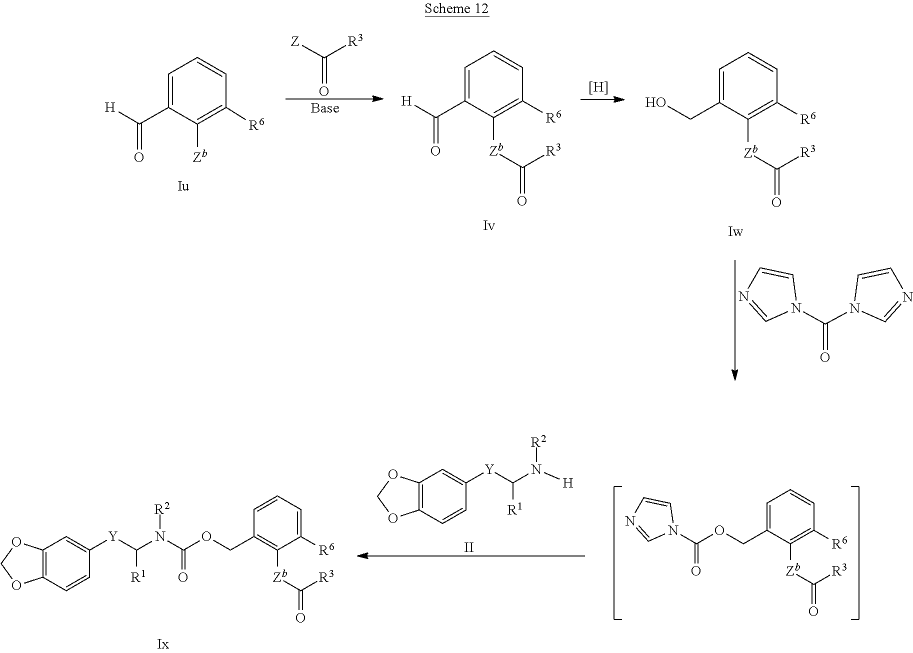

The phosphonate prodrug of Formula Iaa may be prepared by coupling the requisite amine II with a benzylic alcohol Iz as presented in Scheme 13 below and following an analogous assemblage sequence as described previously in Scheme 11. The protected phosphate ly may be obtained by the reaction of a commercially available phenol Iq-1 with a protected phosphate reagent such as di-tert-butylchlorophosphate or di-benzylchlorophosphate in the presence of a base such as triethylamine, i-Pr2NEt or DBU in a solvent such as THF or dichloromethane in the presence of a catalyst such as DMAP. Treatment of the benzaldehyde ly with a reagent such as sodium borohydride in a of solvent such as dichloromethane in the presence of an alcohol such as isopropanol would afford the benzylic alcohol Iz. Carbamate bound formation may be performed by the reaction of the benzylic alcohol with a reagent such as carbonyl diimidazole in a solvent such as dichloromethane, followed by the addition of the amine II. Deprotection of the phosphate to afford Iaa may be performed under acidic conditions (Pg=tert-butyl) using a reagent such as TFA or HCla_{q}. in solvent such as methylene chloride or THF. Unless R⁶ is not compatible with reductive conditions, such as R⁶═NO₂, CN or Br, the deprotection may also be done under hydrogenolysis conditions (Pg=benzyl) using Pd/C as a catalyst in a solvent such as methanol under an atmosphere of H₂.

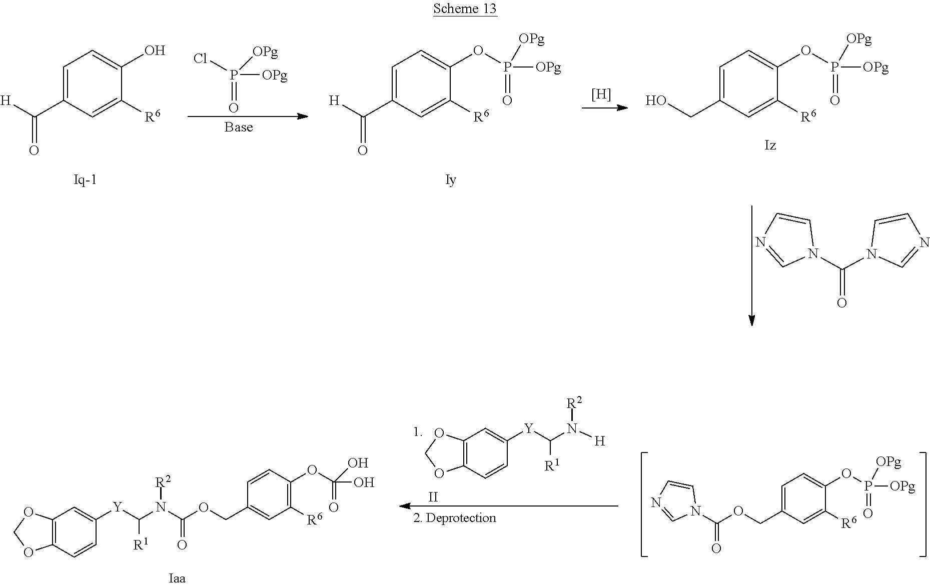

The phosphonate prodrug of Formula Idd may be prepared by coupling the requisite amine II with a benzylic alcohol Icc as presented in Scheme 14 below and following an analogous assemblage sequence as described previously in Scheme 11. The protected phosphate Ibb may be obtained by the reaction of a commercially available phenol lu-1 with a protected phosphate reagent such as di-tert-butylchlorophosphate or di-benzylchlorophosphate in the presence of a base such as triethylamine, i-Pr2NEt or DBU in a solvent such as THF or dichloromethane in the presence of a catalyst such as DMAP. Treatment of the benzaldehyde Ibb with a reagent such as sodium borohydride in a of solvent such as dichloromethane in the presence of an alcohol such as isopropanol would afford the benzylic alcohol Icc. Carbamate bound formation may be performed by the reaction of the benzylic alcohol with a reagent such as carbonyl diimidazole in a solvent such as dichloromethane, followed by the addition of the amine II. Deprotection of the phosphate to afford laa may be performed under acidic conditions (Pg=tert-butyl) using a reagent such as TFA or HCl_{aq}. in solvent such as methylene chloride or THF. Unless R⁶ is not compatible with reductive conditions, such as R⁶═NO₂, CN or Br, the deprotection may also be done under reductive conditions (Pg=benzyl) using Pd/C as a catalyst in a solvent such as methanol under an atmosphere of H₂.

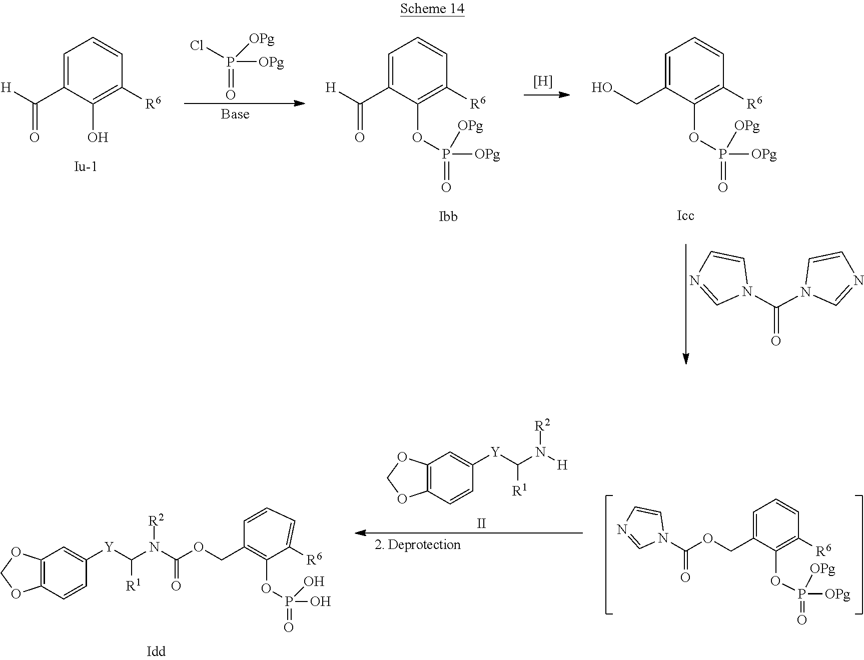

The amide prodrug of Formula Ihh may be prepared by coupling the requisite amine II with the carboxylic acid Igg as presented in Scheme 15 below. Such a carboxylic acid may be generated by a 3-step sequence starting with the phenol lee that may be prepared according to the synthesis reported by Nicolaou M. G. et al. (J. Org. Chem, 1996, 61, 8636). Acylation of lee may be performed by reaction of the phenol with a carboxylic acid (Z═OH) in the presence of a coupling reagent, such as N,N-dicyclohexylcarbodiimide (DCC), N-ethyl-N′-(3-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU) or other similar reagents well known to one skilled in the art. Alternatively, the phenol lee may also be reacted with an acid chloride (Z=Cl) or with an anhydride (Z=—OC(O)R³ or —OC(O)t-Butyl) in the presence of a base such as diisopropylethylamine (DIPEA), triethylamine, 4-methylmorpholine, NaHCO₃, K₂CO₃ or 2,6-lutidine in an appropriate solvent such as methylene chloride, THF, DMF, acetonitrile, or toluene. Deprotection of Iff may be accomplished under mild acidic conditions (Pg=TBS) using a reagent such as PPTS in a solvent such as methanol (Crouch, R. D. Tetrahedron, 2013, 69, 2383) or under reductive conditions (Pg=benzyl) using Pd/C as a catalyst in a solvent such as methanol under an atmosphere of H₂. The corresponding primary alcohol may be oxidized using a reagent such as Jones' reagent in a solvent such as acetone to afford the carboxylic acid Igg which may then be coupled with the amine II in the presence of a coupling reagent as described above.

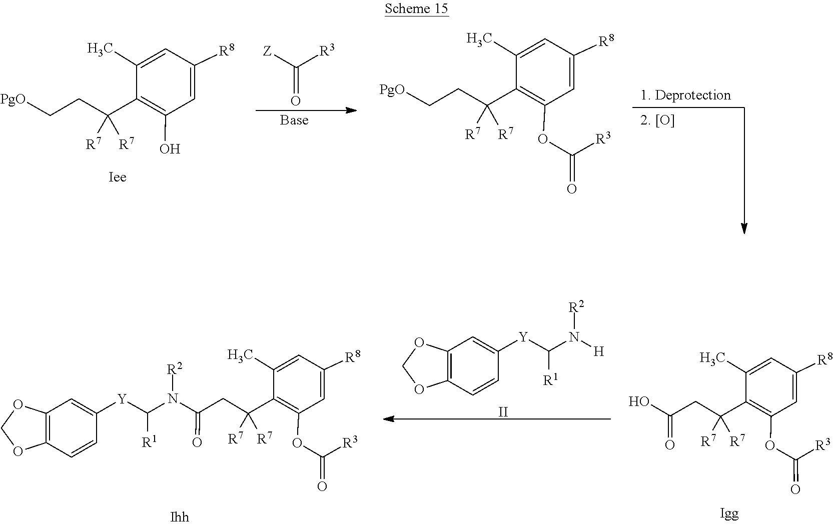

The phosphonate prodrug of Formula Ijj may be prepared by coupling the requisite amine II with the carboxylic acid Iii as depicted in Scheme 16 below. Such a carboxylic acid may be obtained according to the synthesis reported by Nicolaou M.G. et al. (J. Org. Chem, 1996, 61, 8636). Amide bound formation may be performed by reaction of requisite amine II with the carboxylic acid Iii in the presence of a coupling reagent, such as N,N-dicyclohexylcarbodiimide (DCC), N-ethyl-N′-(3-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU) or other similar reagents well known to one skilled in the art. The phosphonate prodrug Ijj may be obtained by deprotection of the corresponding di-benzylphosphate under reductive conditions using Pd/C as a catalyst in a solvent such as methanol under an atmosphere of H₂.

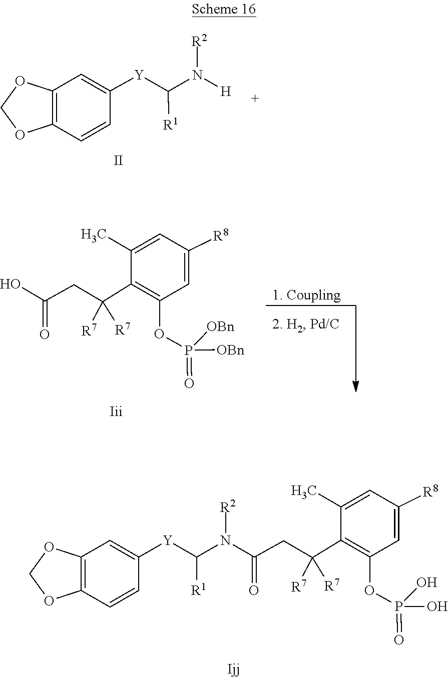

The amide prodrug of Formula Inn may be prepared by coupling the requisite amine II with the carboxylic acid Imm as presented in Scheme 17 below. Such a carboxylic acid may be generated by a 4-step sequence starting with the phenol Ikk that may be prepared according to the synthesis reported by Liao Y. and Wang B. (Bioorg. Med. Chem. Lett., 1999, 9, 1795). Acylation of Ikk may be performed by reaction of the phenol with a carboxylic acid (Z=OH) in the presence of a coupling reagent, such as N,N-dicyclohexylcarbodiimide (DCC), N-ethyl-N′-(3-dimethylaminopropyl)-carbodiimide (EDC), 1,1-carbonyldiimidazole (CDI), diisopropylcarbodiimide (DIC), hexafluorophosphate benzotriazole tetramethyl uronium (HBTU) and hexafluorophosphate azabenzotriazole tetramethyl uranium (HATU) or other similar reagents well known to one skilled in the art. Alternatively, the phenol Ikk may also be reacted with an acid chloride (Z=Cl) or with an anhydride (Z=—OC(O)R³ or —OC(O)t-Butyl) in the presence of a base such as diisopropylethylamine (DIPEA), triethylamine, 4-methylmorpholine, NaHCO₃, K₂CO₃ or 2,6-lutidine in an appropriate solvent such as methylene chloride, THF, DMF, acetonitrile, or toluene. Deprotection of ILL may be accomplished under mild acidic conditions (Pg=TBS) using a reagent such as AcOH in a solvent mixture such as THF/H₂O. The corresponding primary alcohol may be oxidized to the carboxylic acid Imm in a 2-step sequence where the alcohol is first oxidized to the aldehyde using a reagent such as MnO₂ in a solvent such as dichloromethane followed by a Kraus type reaction using reagents well known to one skilled in the art. Finally, coupling of the carboxylic acid Imm with the amine II may be performed in the presence of a coupling reagent as described above to afford the prodrug Inn.

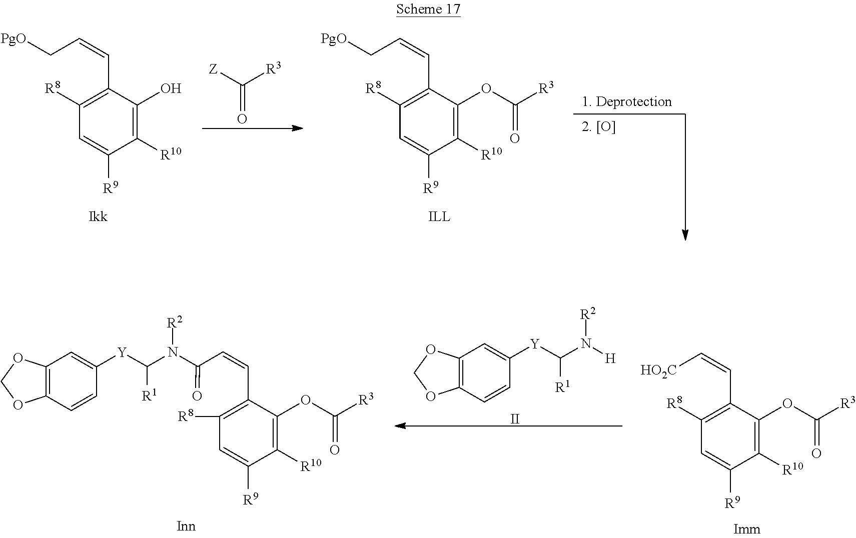

Examples of the compounds of Formula (I) according to the invention include any one of the compounds 1-402 of TABLES 4, 5, and 6; and compounds 403-511 of TABLE 7 below (as well as pharmaceutically acceptable salts of any of these compounds):

TABLE 4

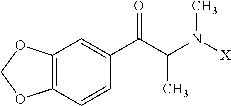

| Cmpd | X |
|---|---|
| 1 | 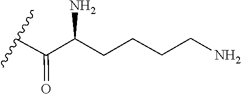 [SMILES: *C(=O)[C@@H](N)CCCCN] |
| 2 | 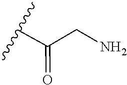 [SMILES: *C(=O)CN] |
| 3 | 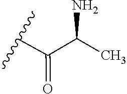 [SMILES: *C(=O)[C@H](C)N] |
| 4 | 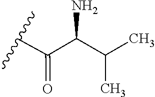 [SMILES: *C(=O)[C@@H](N)C(C)C] |
| 5 | 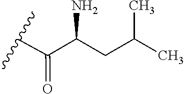 [SMILES: *C(=O)[C@@H](N)CC(C)C] |
| 6 | 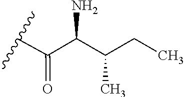 [SMILES: *C(=O)[C@@H](N)[C@@H](C)CC] |
| 7 | 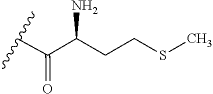 [SMILES: *C(=O)[C@@H](N)CCSC] |
| 8 | 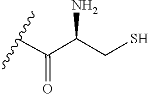 [SMILES: *C(=O)[C@@H](N)CS] |
| 9 | 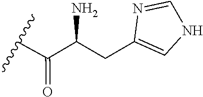 [SMILES: *C(=O)[C@@H](N)Cc1c[nH]cn1] |
| 10 | 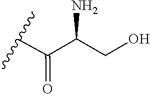 [SMILES: *C(=O)[C@@H](N)CO] |
| 11 | 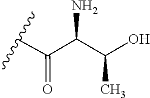 [SMILES: *C(=O)[C@@H](N)[C@H](C)O] |
| 12 | 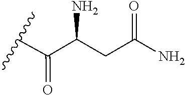 [SMILES: *C(=O)[C@@H](N)CC(N)=O] |
| 13 | 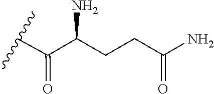 [SMILES: *C(=O)[C@@H](N)CCC(N)=O] |
| 14 | 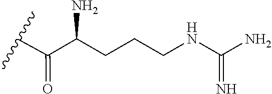 [SMILES: *C(=O)[C@@H](N)CCCNC(=N)N] |
| 15 | 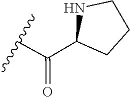 [SMILES: *C(=O)[C@@H]1CCCN1] |
| 16 | 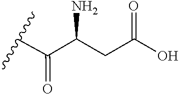 [SMILES: *C(=O)[C@@H](N)CC(=O)O] |
| 17 | 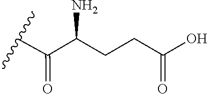 [SMILES: *C(=O)[C@@H](N)CCC(=O)O] |
| 18 | 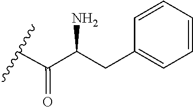 [SMILES: *C(=O)[C@@H](N)Cc1ccccc1] |
| 19 | 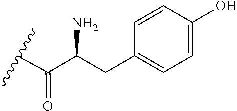 [SMILES: *C(=O)[C@@H](N)Cc1ccc(O)cc1] |
| 20 | 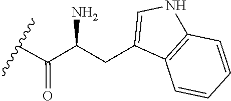 [SMILES: *C(=O)[C@@H](N)Cc1c[nH]c2ccccc12] |
| 21 | 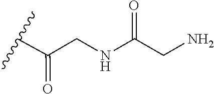 [SMILES: *C(=O)CNC(=O)CN] |
| 22 | 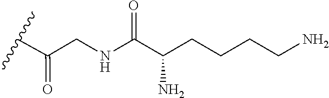 [SMILES: *C(=O)CNC(=O)[C@@H](N)CCCCN] |
| 23 | 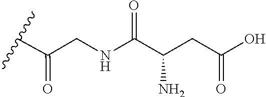 [SMILES: *C(=O)CNC(=O)[C@@H](N)CC(=O)O] |
| 24 | 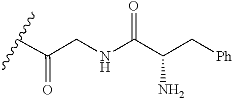 [SMILES: *C(=O)CNC(=O)[C@@H](N)Cc1ccccc1] |
| 25 | 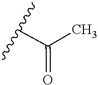 [SMILES: *C(C)=O] |
| 26 | 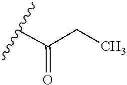 [SMILES: *C(=O)CC] |
| 27 | 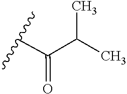 [SMILES: *C(=O)C(C)C] |
| 28 | 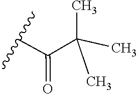 [SMILES: *C(=O)C(C)(C)C] |
| 29 | 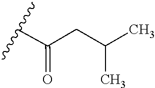 [SMILES: *C(=O)CC(C)C] |
| 30 | 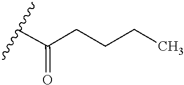 [SMILES: *C(=O)CCCC] |
| 31 | 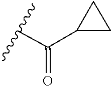 [SMILES: *C(=O)C1CC1] |
| 32 | 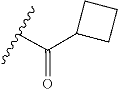 [SMILES: *C(=O)C1CCC1] |
| 33 | 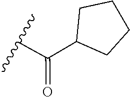 [SMILES: *C(=O)C1CCCC1] |
| 34 | 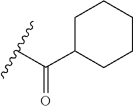 [SMILES: *C(=O)C1CCCCC1] |
| 35 | 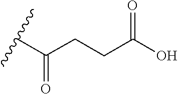 [SMILES: *C(=O)CCC(=O)O] |
| 36 | 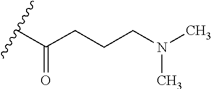 [SMILES: *C(=O)CCCN(C)C] |
| 37 | 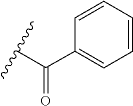 [SMILES: *C(=O)c1ccccc1] |
| 38 | 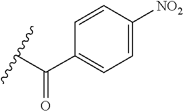 [SMILES: *C(=O)c1ccc([N+](=O)[O-])cc1] |
| 39 | 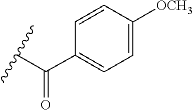 [SMILES: *C(=O)c1ccc(OC)cc1] |
| 40 | 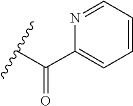 [SMILES: *C(=O)c1ccccn1] |
| 41 | 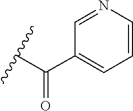 [SMILES: *C(=O)c1cccnc1] |
| 42 | 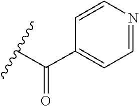 [SMILES: *C(=O)c1ccncc1] |
| 43 | 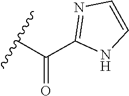 [SMILES: *C(=O)c1ncc[nH]1] |
| 44 | 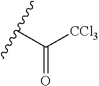 |
| 45 | 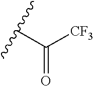 [SMILES: *C(=O)C(F)(F)F] |
| 46 | 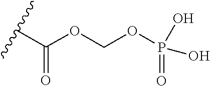 [SMILES: *C(=O)OCOP(=O)(O)O] |
| 47 | 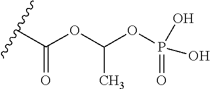 [SMILES: *C(=O)OC(C)OP(=O)(O)O] |
| 48 | 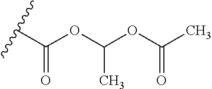 [SMILES: *C(=O)OC(C)OC(C)=O] |
| 49 | 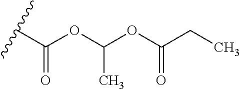 [SMILES: *C(=O)OC(C)OC(=O)CC] |
| 50 | 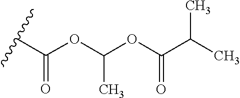 [SMILES: *C(=O)OC(C)OC(=O)C(C)C] |
| 51 | 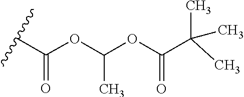 [SMILES: *C(=O)OC(C)OC(=O)C(C)(C)C] |
| 52 | 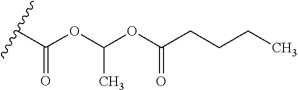 [SMILES: *C(=O)OC(C)OC(=O)CCCC] |
| 53 | 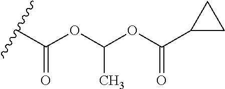 [SMILES: *C(=O)OC(C)OC(=O)C1CC1] |
| 54 | 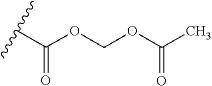 [SMILES: *C(=O)OCOC(C)=O] |
| 55 | 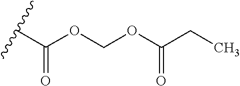 [SMILES: *C(=O)OCOC(=O)CC] |
| 56 | 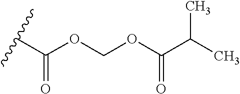 [SMILES: *C(=O)OCOC(=O)C(C)C] |
| 57 | 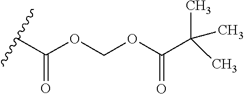 [SMILES: *C(=O)OCOC(=O)C(C)(C)C] |
| 58 | 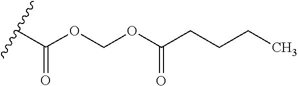 [SMILES: *C(=O)OCOC(=O)CCCC] |
| 59 | 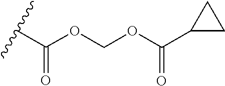 [SMILES: *C(=O)OCOC(=O)C1CC1] |
| 60 | 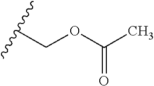 [SMILES: *COC(C)=O] |
| 61 | 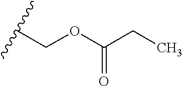 [SMILES: *COC(=O)CC] |
| 62 | 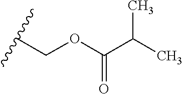 [SMILES: *COC(=O)C(C)C] |
| 63 | 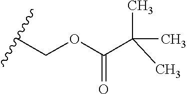 [SMILES: *COC(=O)C(C)(C)C] |
| 64 | 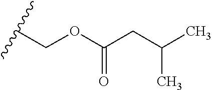 [SMILES: *COC(=O)CC(C)C] |
| 65 | 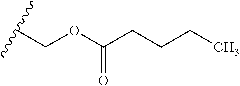 [SMILES: *COC(=O)CCCC] |
| 66 | 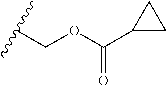 [SMILES: *COC(=O)C1CC1] |
| 67 | 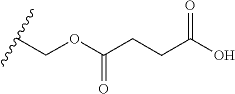 [SMILES: *COC(=O)CCC(=O)O] |
| 68 | 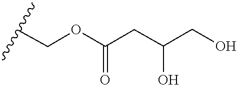 [SMILES: *COC(=O)CC(O)CO] |
| 69 | 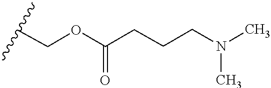 [SMILES: *COC(=O)CCCN(C)C] |
| 70 | CH₂OPO(OH)₂ |
| 71 | 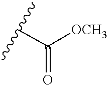 [SMILES: *C(=O)OC] |
| 72 | 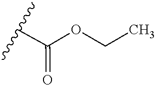 [SMILES: *C(=O)OCC] |
| 73 | 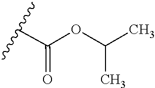 [SMILES: *C(=O)OC(C)C] |
| 74 | 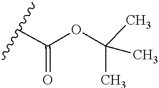 [SMILES: *C(=O)OC(C)(C)C] |
| 75 | 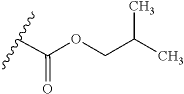 [SMILES: *C(=O)OCC(C)C] |
| 76 | 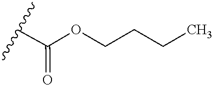 [SMILES: *C(=O)OCCCC] |
| 77 | 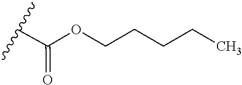 [SMILES: *C(=O)OCCCCC] |
| 78 | 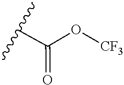 [SMILES: *C(=O)OC(F)(F)F] |
| 79 | 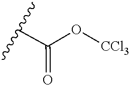 |
| 80 | 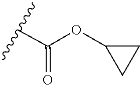 [SMILES: *C(=O)OC1CC1] |
| 81 | 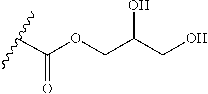 [SMILES: *C(=O)OCC(O)CO] |
| 82 | 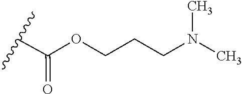 [SMILES: *C(=O)OCCCN(C)C] |
| 83 | 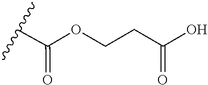 [SMILES: *C(=O)OCCC(=O)O] |
| 84 | PO(OH)₂ |
| 85 | 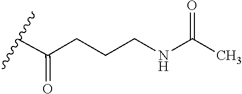 [SMILES: *C(=O)CCCNC(C)=O] |
| 86 | 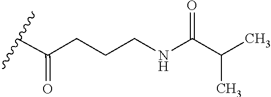 [SMILES: *C(=O)CCCNC(=O)C(C)C] |
| 87 | 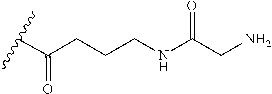 [SMILES: *C(=O)CCCNC(=O)CN] |
| 88 | 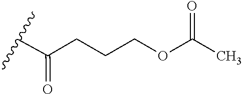 [SMILES: *C(=O)CCCOC(C)=O] |
| 89 | 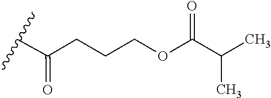 [SMILES: *C(=O)CCCOC(=O)C(C)C] |
| 90 | 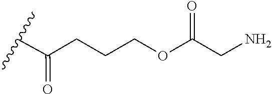 [SMILES: *C(=O)CCCOC(=O)CN] |
| 91 | 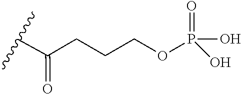 [SMILES: *C(=O)CCCOP(=O)(O)O] |
| 92 | 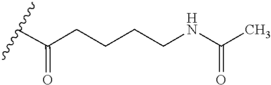 [SMILES: *C(=O)CCCCNC(C)=O] |
| 93 | 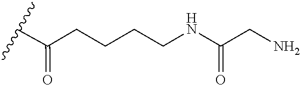 [SMILES: *C(=O)CCCCNC(=O)CN] |
| 94 | 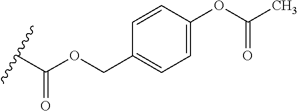 [SMILES: *C(=O)OCc1ccc(OC(C)=O)cc1] |
| 95 | 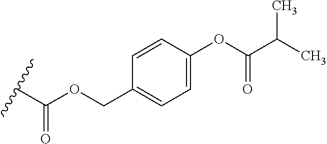 [SMILES: *C(=O)OCc1ccc(OC(=O)C(C)C)cc1] |
| 96 | 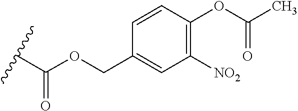 [SMILES: *C(=O)OCc1ccc(OC(C)=O)c([N+](=O)[O-])c1] |
| 97 | 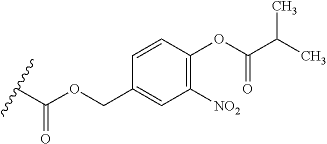 [SMILES: *C(=O)OCc1ccc(OC(=O)C(C)C)c([N+](=O)[O-])c1] |
| 98 | 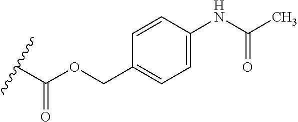 [SMILES: *C(=O)OCc1ccc(NC(C)=O)cc1] |
| 99 | 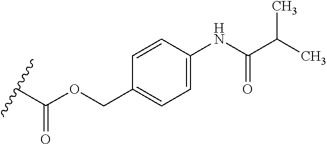 [SMILES: *C(=O)OCc1ccc(NC(=O)C(C)C)cc1] |
| 100 | 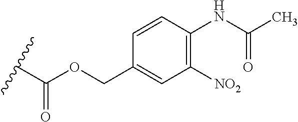 [SMILES: *C(=O)OCc1ccc(NC(C)=O)c([N+](=O)[O-])c1] |
| 101 | 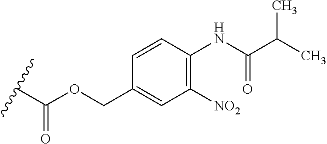 [SMILES: *C(=O)OCc1ccc(NC(=O)C(C)C)c([N+](=O)[O-])c1] |
| 102 | 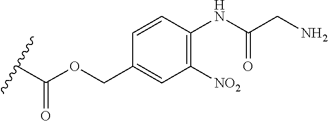 [SMILES: *C(=O)OCc1ccc(NC(=O)CN)c([N+](=O)[O-])c1] |
| 103 | 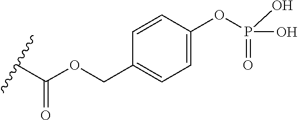 [SMILES: *C(=O)OCc1ccc(OP(=O)(O)O)cc1] |
| 104 | 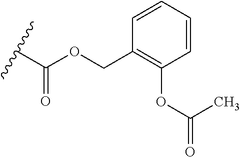 [SMILES: *C(=O)OCc1ccccc1OC(C)=O] |
| 105 | 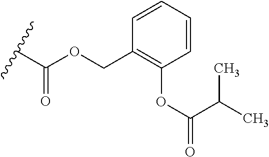 [SMILES: *C(=O)OCc1ccccc1OC(=O)C(C)C] |
| 106 | 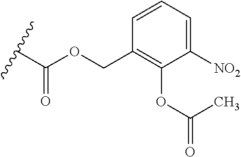 [SMILES: *C(=O)OCc1cccc([N+](=O)[O-])c1OC(C)=O] |
| 107 | 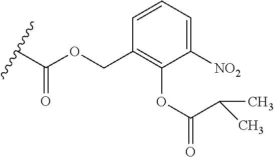 [SMILES: *C(=O)OCc1cccc([N+](=O)[O-])c1OC(=O)C(C)C] |
| 108 | 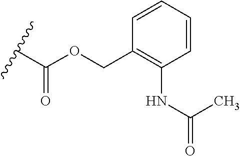 [SMILES: *C(=O)OCc1ccccc1NC(C)=O] |
| 109 | 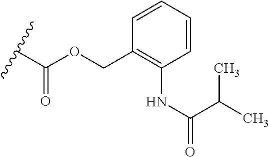 [SMILES: *C(=O)OCc1ccccc1NC(=O)C(C)C] |
| 110 | 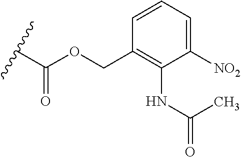 [SMILES: *C(=O)OCc1cccc([N+](=O)[O-])c1NC(C)=O] |
| 111 | 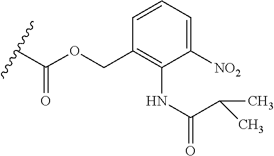 [SMILES: *C(=O)OCc1cccc([N+](=O)[O-])c1NC(=O)C(C)C] |
| 112 | 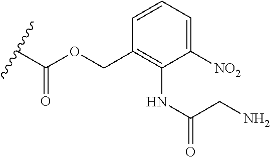 [SMILES: *C(=O)OCc1cccc([N+](=O)[O-])c1NC(=O)CN] |
| 113 | 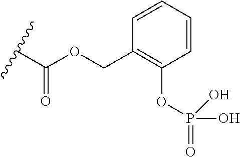 [SMILES: *C(=O)OCc1ccccc1OP(=O)(O)O] |
| 114 | 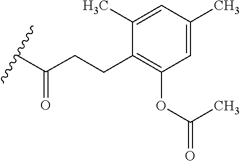 [SMILES: *C(=O)CCc1c(C)cc(C)cc1OC(C)=O] |
| 115 | 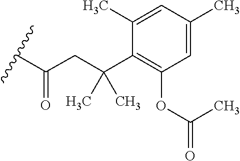 [SMILES: *C(=O)CC(C)(C)c1c(C)cc(C)cc1OC(C)=O] |
| 116 | 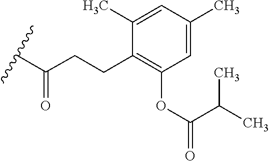 [SMILES: *C(=O)CCc1c(C)cc(C)cc1OC(=O)C(C)C] |
| 117 | 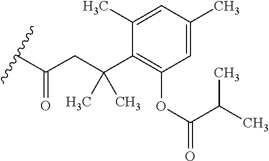 [SMILES: *C(=O)CC(C)(C)c1c(C)cc(C)cc1OC(=O)C(C)C] |
| 118 | 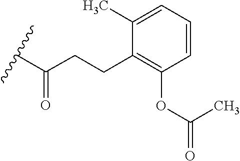 [SMILES: *C(=O)CCc1c(C)cccc1OC(C)=O] |
| 119 | 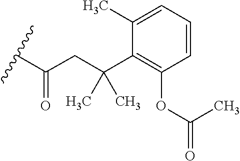 [SMILES: *C(=O)CC(C)(C)c1c(C)cccc1OC(C)=O] |
| 120 | 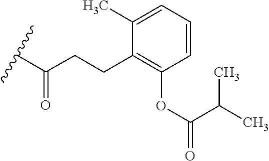 [SMILES: *C(=O)CCc1c(C)cccc1OC(=O)C(C)C] |
| 121 | 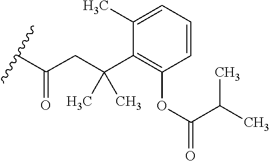 [SMILES: *C(=O)CC(C)(C)c1c(C)cccc1OC(=O)C(C)C] |
| 122 | 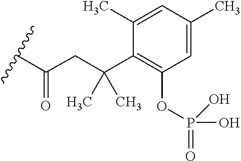 [SMILES: *C(=O)CC(C)(C)c1c(C)cc(C)cc1OP(=O)(O)O] |
| 123 | 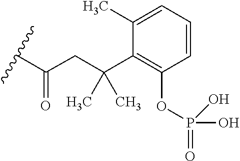 [SMILES: *C(=O)CC(C)(C)c1c(C)cccc1OP(=O)(O)O] |
| 124 | 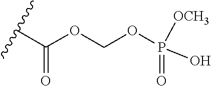 [SMILES: *C(=O)OCOP(=O)(O)OC] |
| 125 | 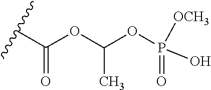 [SMILES: *C(=O)OC(C)OP(=O)(O)OC] |
| 126 | 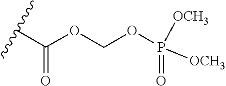 [SMILES: *C(=O)OCOP(=O)(OC)OC] |
| 127 | 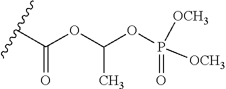 [SMILES: *C(=O)OC(C)OP(=O)(OC)OC] |
| 128 | 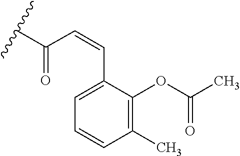 [SMILES: *C(=O)/C=C\c1cccc(C)c1OC(C)=O] |
| 129 | 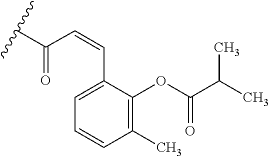 [SMILES: *C(=O)/C=C\c1cccc(C)c1OC(=O)C(C)C] |
| 130 | 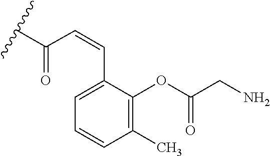 [SMILES: *C(=O)/C=C\c1cccc(C)c1OC(=O)CN] |
| 131 | 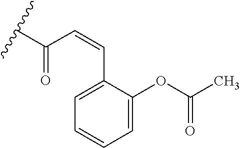 [SMILES: *C(=O)/C=C\c1ccccc1OC(C)=O] |
| 132 | 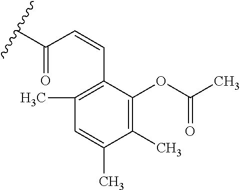 [SMILES: *C(=O)/C=C\c1c(C)cc(C)c(C)c1OC(C)=O] |
| 133 | 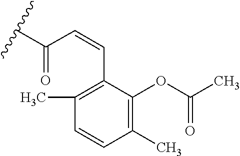 [SMILES: *C(=O)/C=C\c1c(C)ccc(C)c1OC(C)=O] |
| 134 | 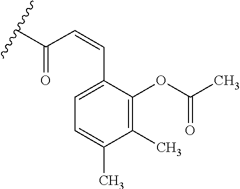 [SMILES: *C(=O)/C=C\c1ccc(C)c(C)c1OC(C)=O] |

TABLE 5

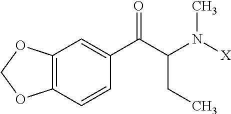

| Cmpd | X |
|---|---|
| 135 | 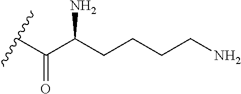 [SMILES: *C(=O)[C@@H](N)CCCCN] |
| 136 | 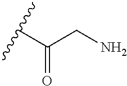 [SMILES: *C(=O)CN] |
| 137 | 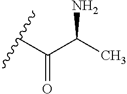 [SMILES: *C(=O)[C@H](C)N] |
| 138 | 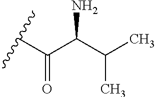 [SMILES: *C(=O)[C@@H](N)C(C)C] |
| 139 | 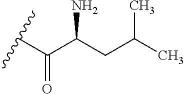 [SMILES: *C(=O)[C@@H](N)CC(C)C] |
| 140 | 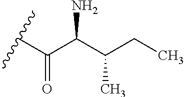 [SMILES: *C(=O)[C@@H](N)[C@@H](C)CC] |
| 141 | 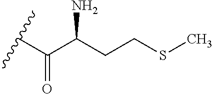 [SMILES: *C(=O)[C@@H](N)CCSC] |
| 142 | 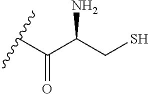 [SMILES: *C(=O)[C@@H](N)CS] |
| 143 | 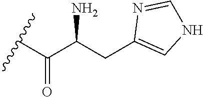 [SMILES: *C(=O)[C@@H](N)Cc1c[nH]cn1] |
| 144 | 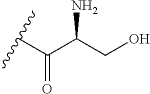 [SMILES: *C(=O)[C@@H](N)CO] |
| 145 | 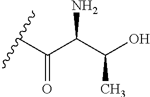 [SMILES: *C(=O)[C@@H](N)[C@H](C)O] |
| 146 | 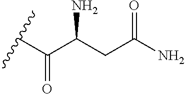 [SMILES: *C(=O)[C@@H](N)CC(N)=O] |
| 147 | 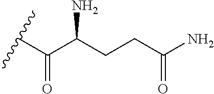 [SMILES: *C(=O)[C@@H](N)CCC(N)=O] |
| 148 | 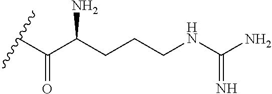 [SMILES: *C(=O)[C@@H](N)CCCNC(=N)N] |
| 149 | 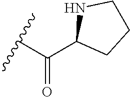 [SMILES: *C(=O)[C@@H]1CCCN1] |
| 150 | 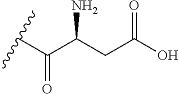 [SMILES: *C(=O)[C@@H](N)CC(=O)O] |
| 151 | 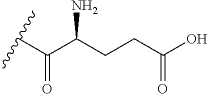 [SMILES: *C(=O)[C@@H](N)CCC(=O)O] |
| 152 | 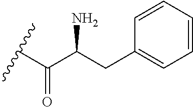 [SMILES: *C(=O)[C@@H](N)Cc1ccccc1] |
| 153 | 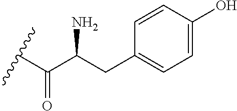 [SMILES: *C(=O)[C@@H](N)Cc1ccc(O)cc1] |
| 154 | 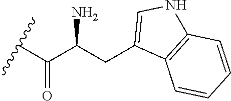 [SMILES: *C(=O)[C@@H](N)Cc1c[nH]c2ccccc12] |
| 155 | 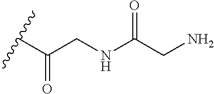 [SMILES: *C(=O)CNC(=O)CN] |
| 156 | 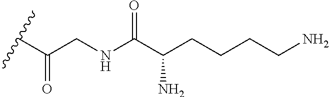 [SMILES: *C(=O)CNC(=O)[C@@H](N)CCCCN] |
| 157 | 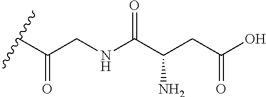 [SMILES: *C(=O)CNC(=O)[C@@H](N)CC(=O)O] |
| 158 | 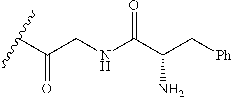 [SMILES: *C(=O)CNC(=O)[C@@H](N)Cc1ccccc1] |
| 159 | 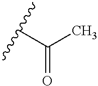 [SMILES: *C(C)=O] |
| 160 | 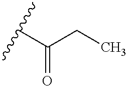 [SMILES: *C(=O)CC] |
| 161 | 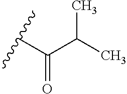 [SMILES: *C(=O)C(C)C] |
| 162 | 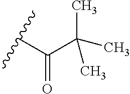 [SMILES: *C(=O)C(C)(C)C] |
| 163 | 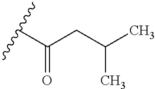 [SMILES: *C(=O)CC(C)C] |
| 164 | 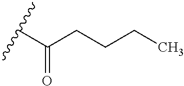 [SMILES: *C(=O)CCCC] |
| 165 | 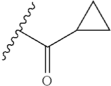 [SMILES: *C(=O)C1CC1] |
| 166 | 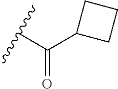 [SMILES: *C(=O)C1CCC1] |
| 167 | 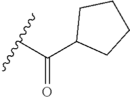 [SMILES: *C(=O)C1CCCC1] |
| 168 | 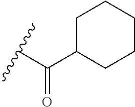 [SMILES: *C(=O)C1CCCCC1] |
| 169 | 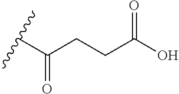 [SMILES: *C(=O)CCC(=O)O] |
| 170 | 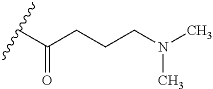 [SMILES: *C(=O)CCCN(C)C] |
| 171 | 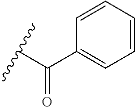 [SMILES: *C(=O)c1ccccc1] |
| 172 | 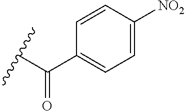 [SMILES: *C(=O)c1ccc([N+](=O)[O-])cc1] |
| 173 | 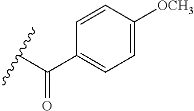 [SMILES: *C(=O)c1ccc(OC)cc1] |
| 174 | 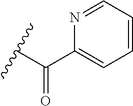 [SMILES: *C(=O)c1ccccn1] |
| 175 | 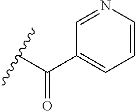 [SMILES: *C(=O)c1cccnc1] |
| 176 | 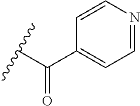 [SMILES: *C(=O)c1ccncc1] |
| 177 | 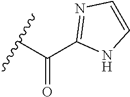 [SMILES: *C(=O)c1ncc[nH]1] |
| 178 | 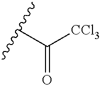 |
| 179 | 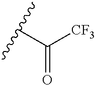 [SMILES: *C(=O)C(F)(F)F] |
| 180 | 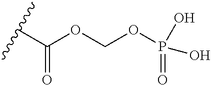 [SMILES: *C(=O)OCOP(=O)(O)O] |
| 181 | 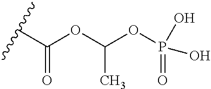 [SMILES: *C(=O)OC(C)OP(=O)(O)O] |
| 182 | 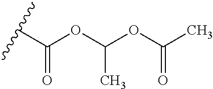 [SMILES: *C(=O)OC(C)OC(C)=O] |
| 183 | 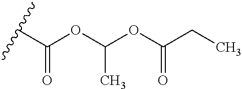 [SMILES: *C(=O)OC(C)OC(=O)CC] |
| 184 | 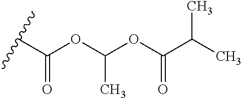 [SMILES: *C(=O)OC(C)OC(=O)C(C)C] |
| 185 | 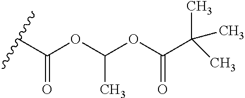 [SMILES: *C(=O)OC(C)OC(=O)C(C)(C)C] |
| 186 | 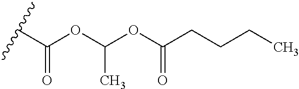 [SMILES: *C(=O)OC(C)OC(=O)CCCC] |
| 187 | 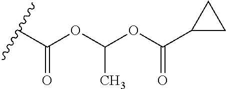 [SMILES: *C(=O)OC(C)OC(=O)C1CC1] |
| 188 | 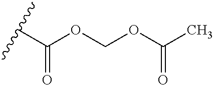 [SMILES: *C(=O)OCOC(C)=O] |
| 189 | 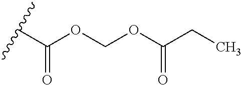 [SMILES: *C(=O)OCOC(=O)CC] |
| 190 | 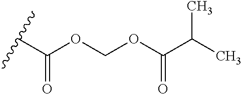 [SMILES: *C(=O)OCOC(=O)C(C)C] |
| 191 | 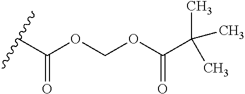 [SMILES: *C(=O)OCOC(=O)C(C)(C)C] |
| 192 | 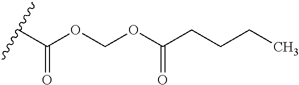 [SMILES: *C(=O)OCOC(=O)CCCC] |
| 193 | 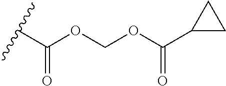 [SMILES: *C(=O)OCOC(=O)C1CC1] |
| 194 | 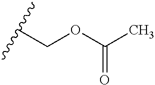 [SMILES: *COC(C)=O] |
| 195 | 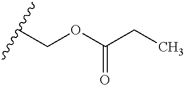 [SMILES: *COC(=O)CC] |
| 196 | 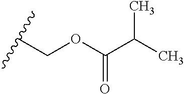 [SMILES: *COC(=O)C(C)C] |
| 197 | 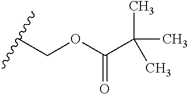 [SMILES: *COC(=O)C(C)(C)C] |
| 198 | 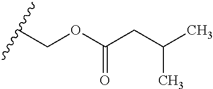 [SMILES: *COC(=O)CC(C)C] |
| 199 | 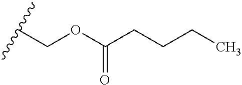 [SMILES: *COC(=O)CCCC] |
| 200 | 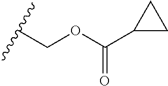 [SMILES: *COC(=O)C1CC1] |
| 201 | 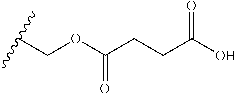 [SMILES: *COC(=O)CCC(=O)O] |
| 202 | 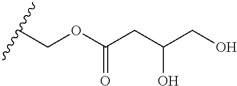 [SMILES: *COC(=O)CC(O)CO] |
| 203 | 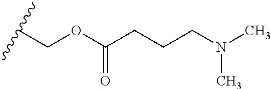 [SMILES: *COC(=O)CCCN(C)C] |
| 204 | CH₂OPO(OH)₂ |
| 205 | 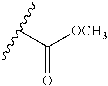 [SMILES: *C(=O)OC] |
| 206 | 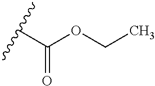 [SMILES: *C(=O)OCC] |
| 207 | 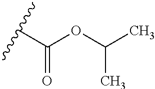 [SMILES: *C(=O)OC(C)C] |
| 208 | 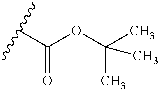 [SMILES: *C(=O)OC(C)(C)C] |
| 209 | 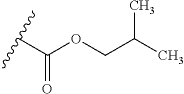 [SMILES: *C(=O)OCC(C)C] |
| 210 | 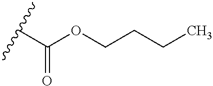 [SMILES: *C(=O)OCCCC] |
| 211 | 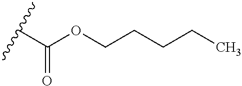 [SMILES: *C(=O)OCCCCC] |
| 212 | 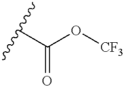 [SMILES: *C(=O)OC(F)(F)F] |
| 213 | 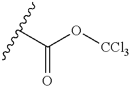 |
| 214 | 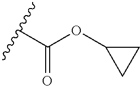 [SMILES: *C(=O)OC1CC1] |
| 215 | 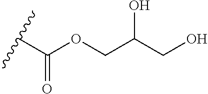 [SMILES: *C(=O)OCC(O)CO] |
| 216 | 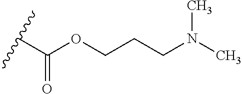 [SMILES: *C(=O)OCCCN(C)C] |
| 217 | 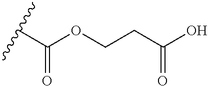 [SMILES: *C(=O)OCCC(=O)O] |
| 218 | PO(OH)₂ |
| 219 | 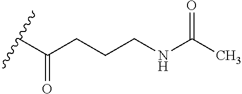 [SMILES: *C(=O)CCCNC(C)=O] |
| 220 | 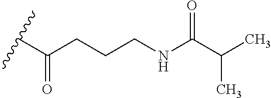 [SMILES: *C(=O)CCCNC(=O)C(C)C] |
| 221 | 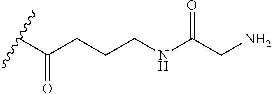 [SMILES: *C(=O)CCCNC(=O)CN] |
| 222 | 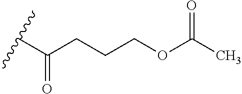 [SMILES: *C(=O)CCCOC(C)=O] |
| 223 | 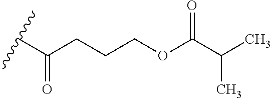 [SMILES: *C(=O)CCCOC(=O)C(C)C] |
| 224 | 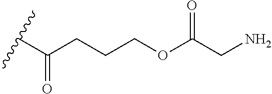 [SMILES: *C(=O)CCCOC(=O)CN] |
| 225 | 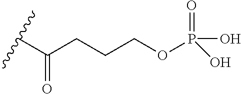 [SMILES: *C(=O)CCCOP(=O)(O)O] |
| 226 | 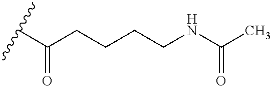 [SMILES: *C(=O)CCCCNC(C)=O] |
| 227 | 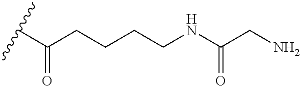 [SMILES: *C(=O)CCCCNC(=O)CN] |
| 228 | 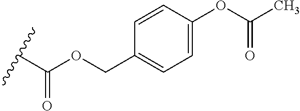 [SMILES: *C(=O)OCc1ccc(OC(C)=O)cc1] |
| 229 | 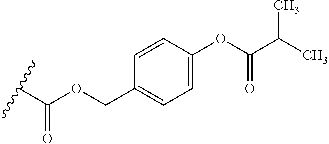 [SMILES: *C(=O)OCc1ccc(OC(=O)C(C)C)cc1] |
| 230 | 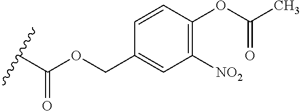 [SMILES: *C(=O)OCc1ccc(OC(C)=O)c([N+](=O)[O-])c1] |
| 231 | 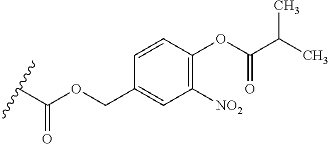 [SMILES: *C(=O)OCc1ccc(OC(=O)C(C)C)c([N+](=O)[O-])c1] |
| 232 | 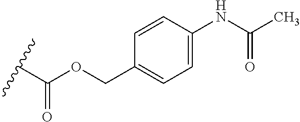 [SMILES: *C(=O)OCc1ccc(NC(C)=O)cc1] |
| 233 | 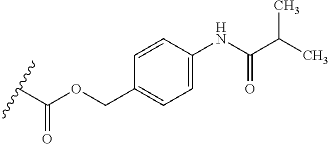 [SMILES: *C(=O)OCc1ccc(NC(=O)C(C)C)cc1] |
| 234 | 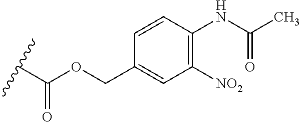 [SMILES: *C(=O)OCc1ccc(NC(C)=O)c([N+](=O)[O-])c1] |
| 235 | 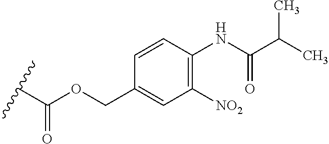 [SMILES: *C(=O)OCc1ccc(NC(=O)C(C)C)c([N+](=O)[O-])c1] |
| 236 | 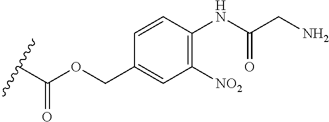 [SMILES: *C(=O)OCc1ccc(NC(=O)CN)c([N+](=O)[O-])c1] |
| 237 | 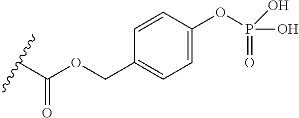 [SMILES: *C(=O)OCc1ccc(OP(=O)(O)O)cc1] |
| 238 | 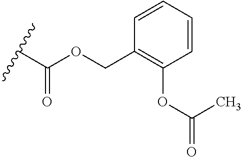 [SMILES: *C(=O)OCc1ccccc1OC(C)=O] |
| 239 | 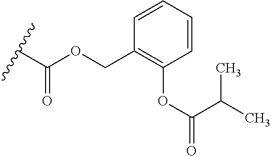 [SMILES: *C(=O)OCc1ccccc1OC(=O)C(C)C] |
| 240 | 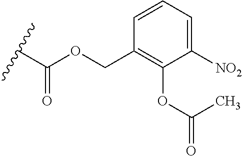 [SMILES: *C(=O)OCc1cccc([N+](=O)[O-])c1OC(C)=O] |
| 241 | 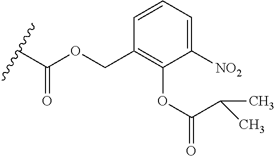 [SMILES: *C(=O)OCc1cccc([N+](=O)[O-])c1OC(=O)C(C)C] |
| 242 | 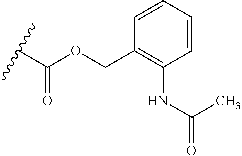 [SMILES: *C(=O)OCc1ccccc1NC(C)=O] |
| 243 | 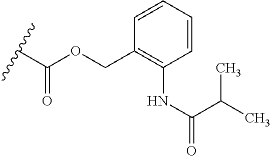 [SMILES: *C(=O)OCc1ccccc1NC(=O)C(C)C] |
| 244 | 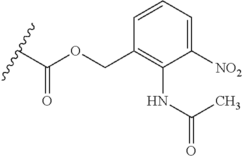 [SMILES: *C(=O)OCc1cccc([N+](=O)[O-])c1NC(C)=O] |
| 245 | 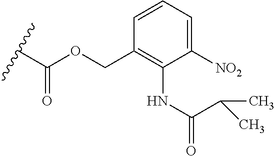 [SMILES: *C(=O)OCc1cccc([N+](=O)[O-])c1NC(=O)C(C)C] |
| 246 | 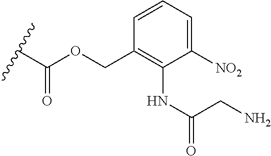 [SMILES: *C(=O)OCc1cccc([N+](=O)[O-])c1NC(=O)CN] |
| 247 | 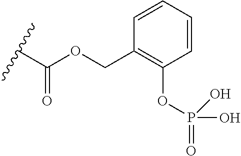 [SMILES: *C(=O)OCc1ccccc1OP(=O)(O)O] |
| 248 | 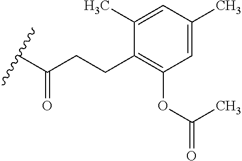 [SMILES: *C(=O)CCc1c(C)cc(C)cc1OC(C)=O] |
| 249 | 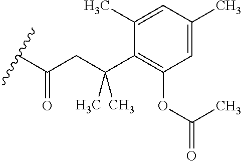 [SMILES: *C(=O)CC(C)(C)c1c(C)cc(C)cc1OC(C)=O] |
| 250 | 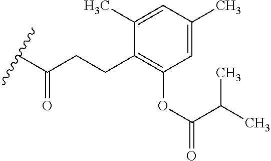 [SMILES: *C(=O)CCc1c(C)cc(C)cc1OC(=O)C(C)C] |
| 251 | 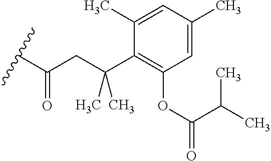 [SMILES: *C(=O)CC(C)(C)c1c(C)cc(C)cc1OC(=O)C(C)C] |
| 252 | 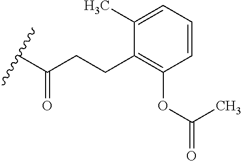 [SMILES: *C(=O)CCc1c(C)cccc1OC(C)=O] |
| 253 | 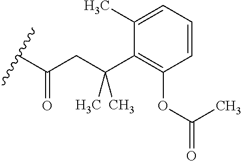 [SMILES: *C(=O)CC(C)(C)c1c(C)cccc1OC(C)=O] |
| 254 | 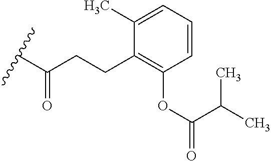 [SMILES: *C(=O)CCc1c(C)cccc1OC(=O)C(C)C] |
| 255 | 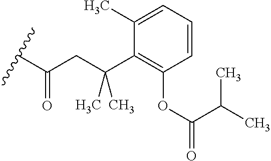 [SMILES: *C(=O)CC(C)(C)c1c(C)cccc1OC(=O)C(C)C] |
| 256 | 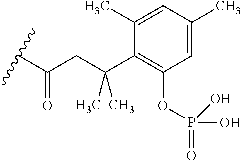 [SMILES: *C(=O)CC(C)(C)c1c(C)cc(C)cc1OP(=O)(O)O] |
| 257 | 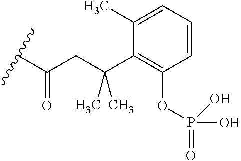 [SMILES: *C(=O)CC(C)(C)c1c(C)cccc1OP(=O)(O)O] |
| 258 | 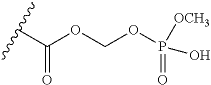 [SMILES: *C(=O)OCOP(=O)(O)OC] |
| 259 | 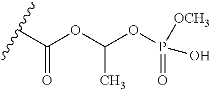 [SMILES: *C(=O)OC(C)OP(=O)(O)OC] |
| 260 | 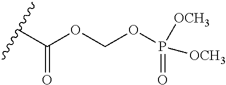 [SMILES: *C(=O)OCOP(=O)(OC)OC] |
| 261 | 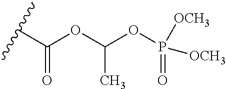 [SMILES: *C(=O)OC(C)OP(=O)(OC)OC] |
| 262 | 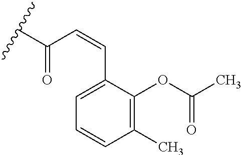 [SMILES: *C(=O)/C=C\c1cccc(C)c1OC(C)=O] |
| 263 | 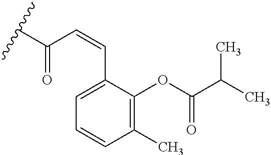 [SMILES: *C(=O)/C=C\c1cccc(C)c1OC(=O)C(C)C] |
| 264 | 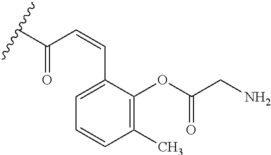 [SMILES: *C(=O)/C=C\c1cccc(C)c1OC(=O)CN] |
| 265 | 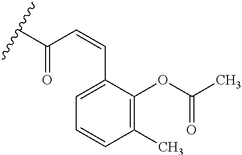 [SMILES: *C(=O)/C=C\c1cccc(C)c1OC(C)=O] |
| 266 | 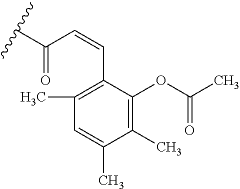 [SMILES: *C(=O)/C=C\c1c(C)cc(C)c(C)c1OC(C)=O] |
| 267 | 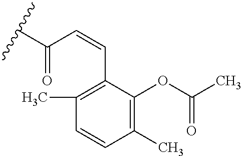 [SMILES: *C(=O)/C=C\c1c(C)ccc(C)c1OC(C)=O] |
| 268 | 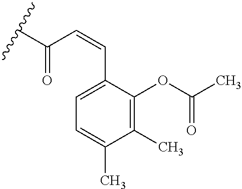 [SMILES: *C(=O)/C=C\c1ccc(C)c(C)c1OC(C)=O] |

TABLE 6

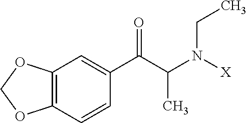

| Cmpd | X |
|---|---|
| 269 | 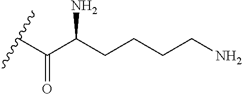 [SMILES: *C(=O)[C@@H](N)CCCCN] |
| 270 | 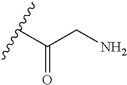 [SMILES: *C(=O)CN] |
| 271 | 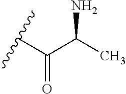 [SMILES: *C(=O)[C@H](C)N] |
| 272 | 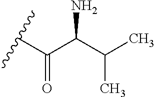 [SMILES: *C(=O)[C@@H](N)C(C)C] |
| 273 | 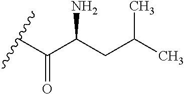 [SMILES: *C(=O)[C@@H](N)CC(C)C] |
| 274 | 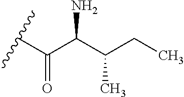 [SMILES: *C(=O)[C@@H](N)[C@@H](C)CC] |
| 275 | 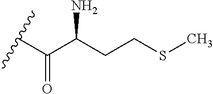 [SMILES: *C(=O)[C@@H](N)CCSC] |
| 276 | 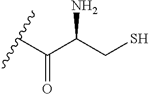 [SMILES: *C(=O)[C@@H](N)CS] |
| 277 | 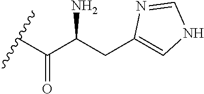 [SMILES: *C(=O)[C@@H](N)Cc1c[nH]cn1] |
| 278 | 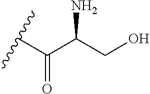 [SMILES: *C(=O)[C@@H](N)CO] |
| 279 | 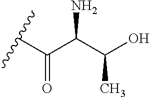 [SMILES: *C(=O)[C@@H](N)[C@H](C)O] |
| 280 | 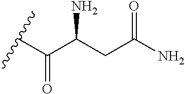 [SMILES: *C(=O)[C@@H](N)CC(N)=O] |
| 281 | 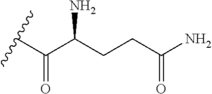 [SMILES: *C(=O)[C@@H](N)CCC(N)=O] |
| 282 | 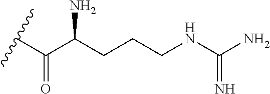 [SMILES: *C(=O)[C@@H](N)CCCNC(=N)N] |
| 283 | 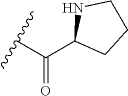 [SMILES: *C(=O)[C@@H]1CCCN1] |
| 284 | 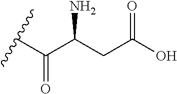 [SMILES: *C(=O)[C@@H](N)CC(=O)O] |
| 285 | 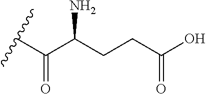 [SMILES: *C(=O)[C@@H](N)CCC(=O)O] |
| 286 | 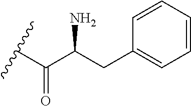 [SMILES: *C(=O)[C@@H](N)Cc1ccccc1] |
| 287 | 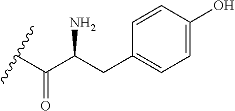 [SMILES: *C(=O)[C@@H](N)Cc1ccc(O)cc1] |
| 288 | 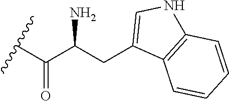 [SMILES: *C(=O)[C@@H](N)Cc1c[nH]c2ccccc12] |
| 289 | 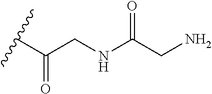 [SMILES: *C(=O)CNC(=O)CN] |
| 290 | 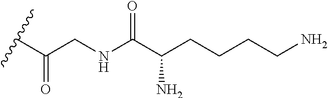 [SMILES: *C(=O)CNC(=O)[C@@H](N)CCCCN] |
| 291 | 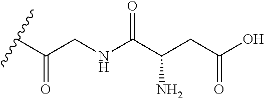 [SMILES: *C(=O)CNC(=O)[C@@H](N)CC(=O)O] |
| 292 | 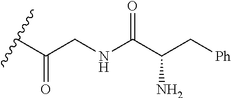 [SMILES: *C(=O)CNC(=O)[C@@H](N)Cc1ccccc1] |
| 293 | 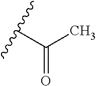 [SMILES: *C(C)=O] |
| 294 | 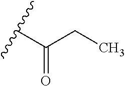 [SMILES: *C(=O)CC] |
| 295 | 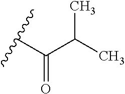 [SMILES: *C(=O)C(C)C] |
| 296 | 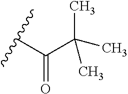 [SMILES: *C(=O)C(C)(C)C] |
| 297 | 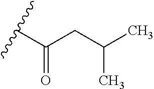 [SMILES: *C(=O)CC(C)C] |
| 298 | 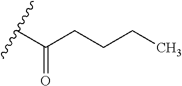 [SMILES: *C(=O)CCCC] |
| 299 | 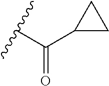 [SMILES: *C(=O)C1CC1] |
| 300 | 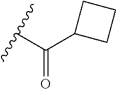 [SMILES: *C(=O)C1CCC1] |
| 301 | 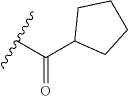 [SMILES: *C(=O)C1CCCC1] |
| 302 | 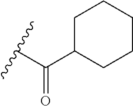 [SMILES: *C(=O)C1CCCCC1] |
| 303 | 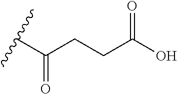 [SMILES: *C(=O)CCC(=O)O] |
| 304 | 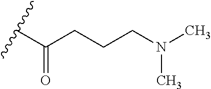 [SMILES: *C(=O)CCCN(C)C] |
| 305 | 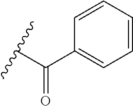 [SMILES: *C(=O)c1ccccc1] |
| 306 | 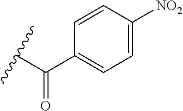 [SMILES: *C(=O)c1ccc([N+](=O)[O-])cc1] |
| 307 | 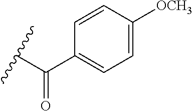 [SMILES: *C(=O)c1ccc(OC)cc1] |
| 308 | 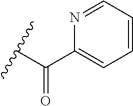 [SMILES: *C(=O)c1ccccn1] |
| 309 | 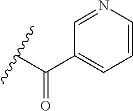 [SMILES: *C(=O)c1cccnc1] |
| 310 | 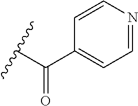 [SMILES: *C(=O)c1ccncc1] |
| 311 | 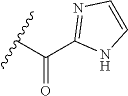 [SMILES: *C(=O)c1ncc[nH]1] |
| 312 | 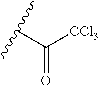 |
| 313 | 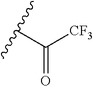 [SMILES: *C(=O)C(F)(F)F] |
| 314 | 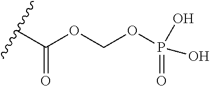 [SMILES: *C(=O)OCOP(=O)(O)O] |
| 315 | 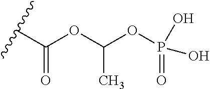 [SMILES: *C(=O)OC(C)OP(=O)(O)O] |
| 316 | 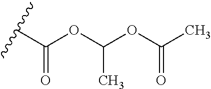 [SMILES: *C(=O)OC(C)OC(C)=O] |
| 317 | 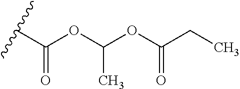 [SMILES: *C(=O)OC(C)OC(=O)CC] |
| 318 | 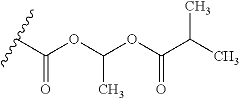 [SMILES: *C(=O)OC(C)OC(=O)C(C)C] |
| 319 | 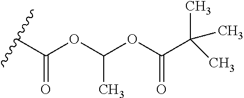 [SMILES: *C(=O)OC(C)OC(=O)C(C)(C)C] |
| 320 | 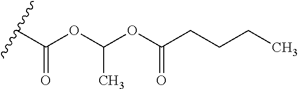 [SMILES: *C(=O)OC(C)OC(=O)CCCC] |
| 321 | 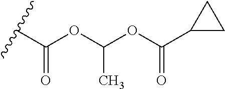 [SMILES: *C(=O)OC(C)OC(=O)C1CC1] |
| 322 | 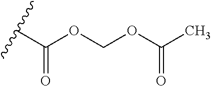 [SMILES: *C(=O)OCOC(C)=O] |
| 323 | 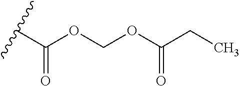 [SMILES: *C(=O)OCOC(=O)CC] |
| 324 | 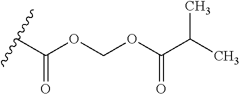 [SMILES: *C(=O)OCOC(=O)C(C)C] |
| 325 | 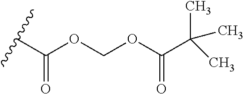 [SMILES: *C(=O)OCOC(=O)C(C)(C)C] |
| 326 | 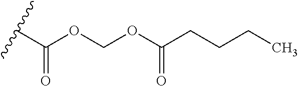 [SMILES: *C(=O)OCOC(=O)CCCC] |
| 327 | 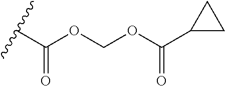 [SMILES: *C(=O)OCOC(=O)C1CC1] |
| 328 | 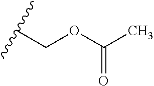 [SMILES: *COC(C)=O] |
| 329 | 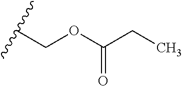 [SMILES: *COC(=O)CC] |
| 330 | 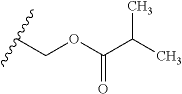 [SMILES: *COC(=O)C(C)C] |
| 331 | 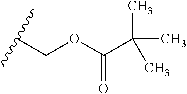 [SMILES: *COC(=O)C(C)(C)C] |
| 332 | 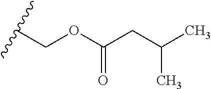 [SMILES: *COC(=O)CC(C)C] |
| 333 | 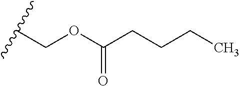 [SMILES: *COC(=O)CCCC] |
| 334 | 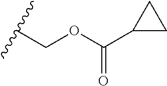 [SMILES: *COC(=O)C1CC1] |
| 335 | 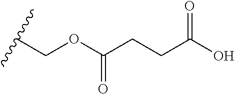 [SMILES: *COC(=O)CCC(=O)O] |
| 336 | 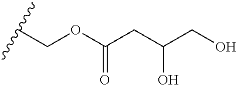 [SMILES: *COC(=O)CC(O)CO] |
| 337 | 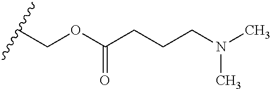 [SMILES: *COC(=O)CCCN(C)C] |
| 338 | CH₂OPO(OH)₂ |
| 339 | 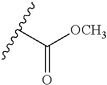 [SMILES: *C(=O)OC] |
| 340 | 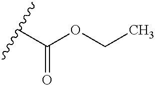 [SMILES: *C(=O)OCC] |
| 341 | 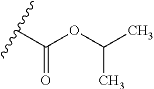 [SMILES: *C(=O)OC(C)C] |
| 342 | 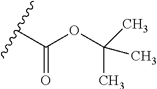 [SMILES: *C(=O)OC(C)(C)C] |
| 343 | 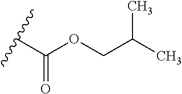 [SMILES: *C(=O)OCC(C)C] |
| 344 | 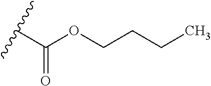 [SMILES: *C(=O)OCCCC] |
| 345 | 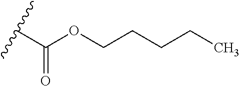 [SMILES: *C(=O)OCCCCC] |
| 346 | 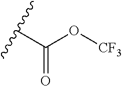 [SMILES: *C(=O)OC(F)(F)F] |
| 347 | 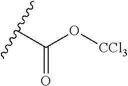 |
| 348 | 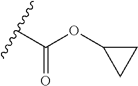 [SMILES: *C(=O)OC1CC1] |
| 349 | 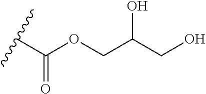 [SMILES: *C(=O)OCC(O)CO] |
| 350 | 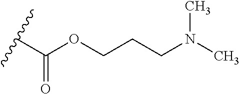 [SMILES: *C(=O)OCCCN(C)C] |
| 351 | 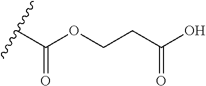 [SMILES: *C(=O)OCCC(=O)O] |
| 352 | PO(OH)₂ |
| 353 | 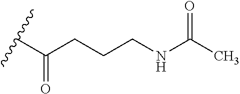 [SMILES: *C(=O)CCCNC(C)=O] |
| 354 | 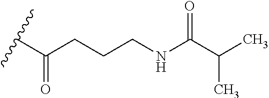 [SMILES: *C(=O)CCCNC(=O)C(C)C] |
| 355 | 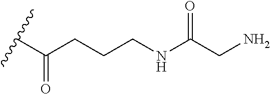 [SMILES: *C(=O)CCCNC(=O)CN] |
| 356 | 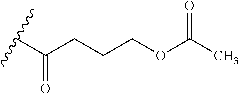 [SMILES: *C(=O)CCCOC(C)=O] |
| 357 | 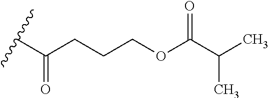 [SMILES: *C(=O)CCCOC(=O)C(C)C] |
| 358 | 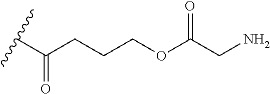 [SMILES: *C(=O)CCCOC(=O)CN] |
| 359 | 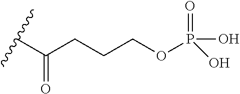 [SMILES: *C(=O)CCCOP(=O)(O)O] |
| 360 | 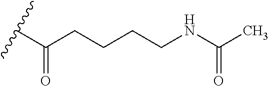 [SMILES: *C(=O)CCCCNC(C)=O] |
| 361 | 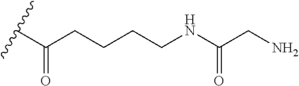 [SMILES: *C(=O)CCCCNC(=O)CN] |
| 362 | 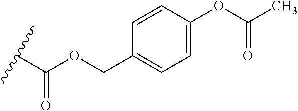 [SMILES: *C(=O)OCc1ccc(OC(C)=O)cc1] |
| 363 | 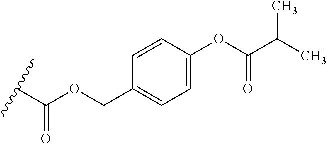 [SMILES: *C(=O)OCc1ccc(OC(=O)C(C)C)cc1] |
| 364 | 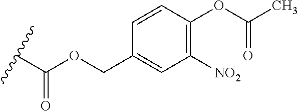 [SMILES: *C(=O)OCc1ccc(OC(C)=O)c([N+](=O)[O-])c1] |
| 365 | 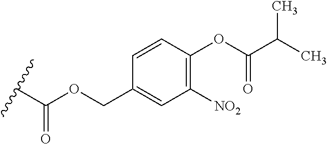 [SMILES: *C(=O)OCc1ccc(OC(=O)C(C)C)c([N+](=O)[O-])c1] |
| 366 | 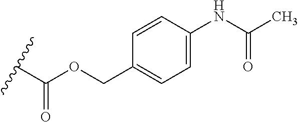 [SMILES: *C(=O)OCc1ccc(NC(C)=O)cc1] |
| 367 | 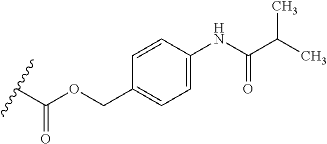 [SMILES: *C(=O)OCc1ccc(NC(=O)C(C)C)cc1] |
| 368 | 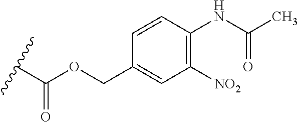 [SMILES: *C(=O)OCc1ccc(NC(C)=O)c([N+](=O)[O-])c1] |
| 369 | 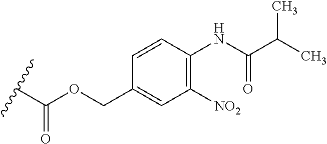 [SMILES: *C(=O)OCc1ccc(NC(=O)C(C)C)c([N+](=O)[O-])c1] |
| 370 | 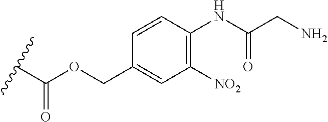 [SMILES: *C(=O)OCc1ccc(NC(=O)CN)c([N+](=O)[O-])c1] |
| 371 | 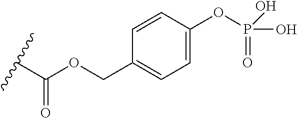 [SMILES: *C(=O)OCc1ccc(OP(=O)(O)O)cc1] |
| 372 | 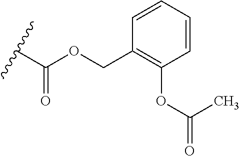 [SMILES: *C(=O)OCc1ccccc1OC(C)=O] |
| 373 | 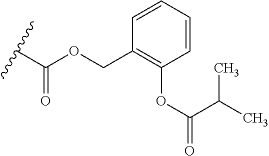 [SMILES: *C(=O)OCc1ccccc1OC(=O)C(C)C] |
| 374 | 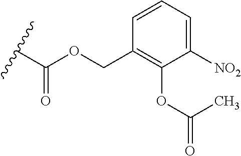 [SMILES: *C(=O)OCc1cccc([N+](=O)[O-])c1OC(C)=O] |
| 375 | 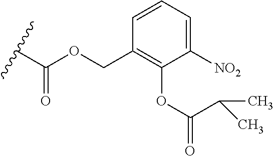 [SMILES: *C(=O)OCc1cccc([N+](=O)[O-])c1OC(=O)C(C)C] |
| 376 | 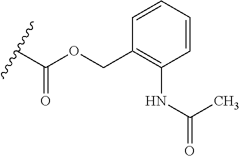 [SMILES: *C(=O)OCc1ccccc1NC(C)=O] |
| 377 | 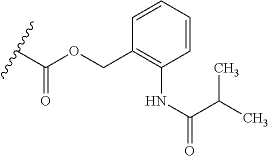 [SMILES: *C(=O)OCc1ccccc1NC(=O)C(C)C] |
| 378 | 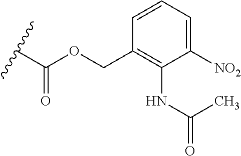 [SMILES: *C(=O)OCc1cccc([N+](=O)[O-])c1NC(C)=O] |
| 379 | 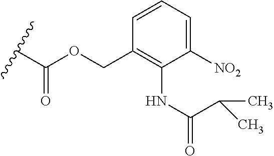 [SMILES: *C(=O)OCc1cccc([N+](=O)[O-])c1NC(=O)C(C)C] |
| 380 | 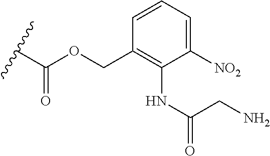 [SMILES: *C(=O)OCc1cccc([N+](=O)[O-])c1NC(=O)CN] |
| 381 | 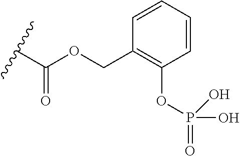 [SMILES: *C(=O)OCc1ccccc1OP(=O)(O)O] |
| 382 | 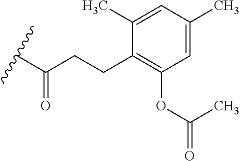 [SMILES: *C(=O)CCc1c(C)cc(C)cc1OC(C)=O] |
| 383 | 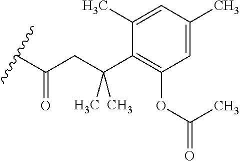 [SMILES: *C(=O)CC(C)(C)c1c(C)cc(C)cc1OC(C)=O] |
| 384 | 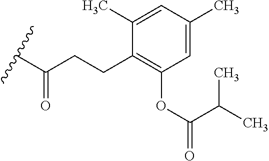 [SMILES: *C(=O)CCc1c(C)cc(C)cc1OC(=O)C(C)C] |
| 385 | 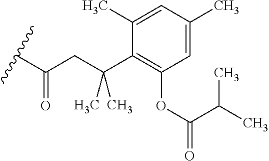 [SMILES: *C(=O)CC(C)(C)c1c(C)cc(C)cc1OC(=O)C(C)C] |
| 386 | 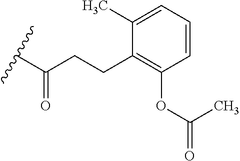 [SMILES: *C(=O)CCc1c(C)cccc1OC(C)=O] |
| 387 | 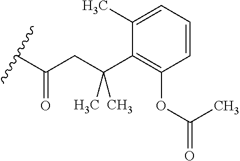 [SMILES: *C(=O)CC(C)(C)c1c(C)cccc1OC(C)=O] |
| 388 | 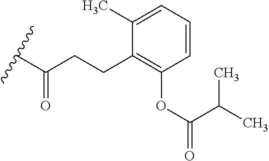 [SMILES: *C(=O)CCc1c(C)cccc1OC(=O)C(C)C] |
| 389 | 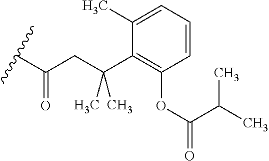 [SMILES: *C(=O)CC(C)(C)c1c(C)cccc1OC(=O)C(C)C] |
| 390 | 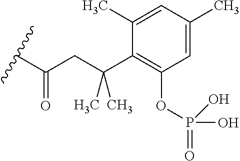 [SMILES: *C(=O)CC(C)(C)c1c(C)cc(C)cc1OP(=O)(O)O] |
| 391 | 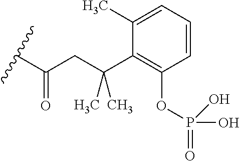 [SMILES: *C(=O)CC(C)(C)c1c(C)cccc1OP(=O)(O)O] |
| 392 | 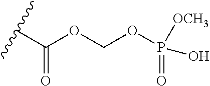 [SMILES: *C(=O)OCOP(=O)(O)OC] |
| 393 | 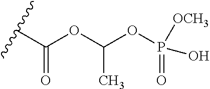 [SMILES: *C(=O)OC(C)OP(=O)(O)OC] |
| 394 | 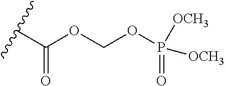 [SMILES: *C(=O)OCOP(=O)(OC)OC] |
| 395 | 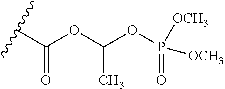 [SMILES: *C(=O)OC(C)OP(=O)(OC)OC] |
| 396 | 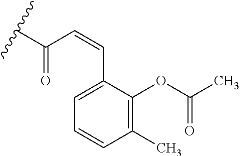 [SMILES: *C(=O)/C=C\c1cccc(C)c1OC(C)=O] |
| 397 | 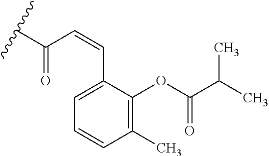 [SMILES: *C(=O)/C=C\c1cccc(C)c1OC(=O)C(C)C] |
| 398 | 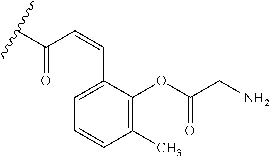 [SMILES: *C(=O)/C=C\c1cccc(C)c1OC(=O)CN] |
| 399 | 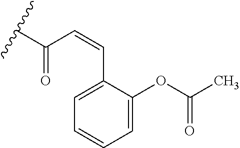 [SMILES: *C(=O)/C=C\c1ccccc1OC(C)=O] |
| 400 | 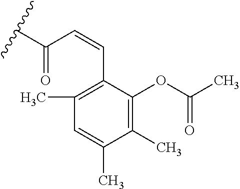 [SMILES: *C(=O)/C=C\c1c(C)cc(C)c(C)c1OC(C)=O] |
| 401 | 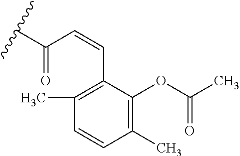 [SMILES: *C(=O)/C=C\c1c(C)ccc(C)c1OC(C)=O] |
| 402 | 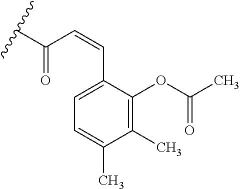 [SMILES: *C(=O)/C=C\c1ccc(C)c(C)c1OC(C)=O] |

TABLE 7

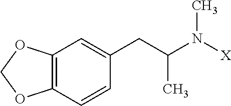

| Cmpd | X |
|---|---|
| 403 | 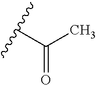 [SMILES: *C(C)=O] |
| 404 | 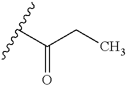 [SMILES: *C(=O)CC] |
| 405 | 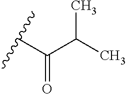 [SMILES: *C(=O)C(C)C] |
| 406 | 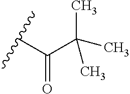 [SMILES: *C(=O)C(C)(C)C] |
| 407 | 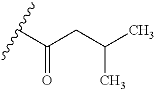 [SMILES: *C(=O)CC(C)C] |
| 408 | 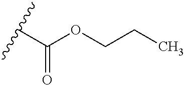 [SMILES: *C(=O)OCCC] |
| 409 | 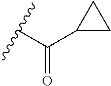 [SMILES: *C(=O)C1CC1] |
| 410 | 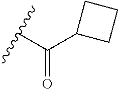 [SMILES: *C(=O)C1CCC1] |
| 411 | 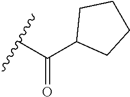 [SMILES: *C(=O)C1CCCC1] |
| 412 | 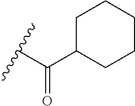 [SMILES: *C(=O)C1CCCCC1] |
| 413 | 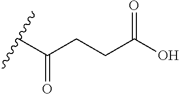 [SMILES: *C(=O)CCC(=O)O] |
| 414 | 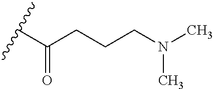 [SMILES: *C(=O)CCCN(C)C] |
| 415 | 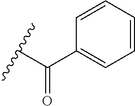 [SMILES: *C(=O)c1ccccc1] |
| 416 | 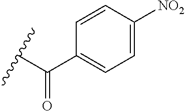 [SMILES: *C(=O)c1ccc([N+](=O)[O-])cc1] |
| 417 | 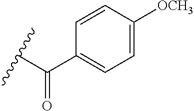 [SMILES: *C(=O)c1ccc(OC)cc1] |
| 418 | 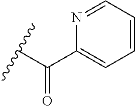 [SMILES: *C(=O)c1ccccn1] |
| 419 | 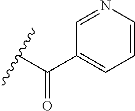 [SMILES: *C(=O)c1cccnc1] |
| 420 | 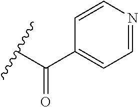 [SMILES: *C(=O)c1ccncc1] |
| 421 | 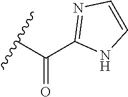 [SMILES: *C(=O)c1ncc[nH]1] |
| 422 | 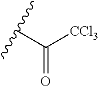 |
| 423 | 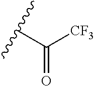 [SMILES: *C(=O)C(F)(F)F] |
| 424 | 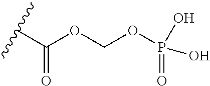 [SMILES: *C(=O)OCOP(=O)(O)O] |
| 425 | 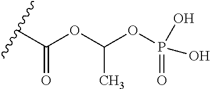 [SMILES: *C(=O)OC(C)OP(=O)(O)O] |
| 426 | 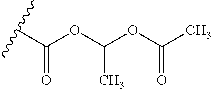 [SMILES: *C(=O)OC(C)OC(C)=O] |
| 427 | 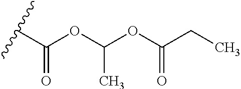 [SMILES: *C(=O)OC(C)OC(=O)CC] |
| 428 | 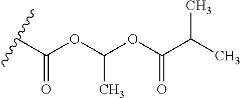 [SMILES: *C(=O)OC(C)OC(=O)C(C)C] |
| 429 | 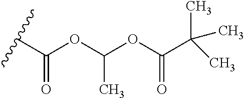 [SMILES: *C(=O)OC(C)OC(=O)C(C)(C)C] |
| 430 | 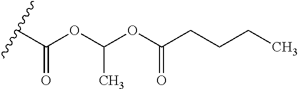 [SMILES: *C(=O)OC(C)OC(=O)CCCC] |
| 431 | 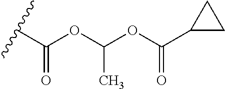 [SMILES: *C(=O)OC(C)OC(=O)C1CC1] |
| 432 | 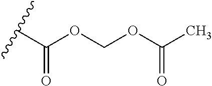 [SMILES: *C(=O)OCOC(C)=O] |
| 433 | 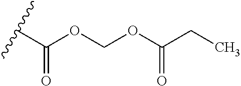 [SMILES: *C(=O)OCOC(=O)CC] |
| 434 | 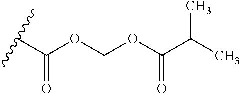 [SMILES: *C(=O)OCOC(=O)C(C)C] |
| 435 | 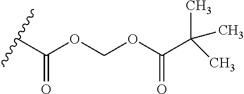 [SMILES: *C(=O)OCOC(=O)C(C)(C)C] |
| 436 | 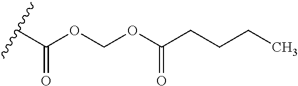 [SMILES: *C(=O)OCOC(=O)CCCC] |
| 437 | 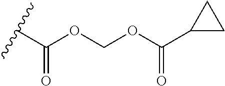 [SMILES: *C(=O)OCOC(=O)C1CC1] |
| 438 | 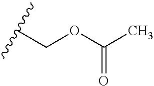 [SMILES: *COC(C)=O] |
| 439 | 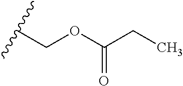 [SMILES: *COC(=O)CC] |
| 440 | 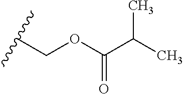 [SMILES: *COC(=O)C(C)C] |
| 441 | 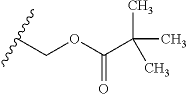 [SMILES: *COC(=O)C(C)(C)C] |
| 442 | 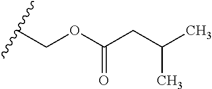 [SMILES: *COC(=O)CC(C)C] |
| 443 | 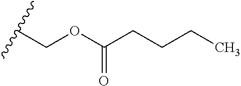 [SMILES: *COC(=O)CCCC] |
| 444 | 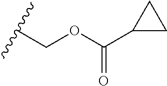 [SMILES: *COC(=O)C1CC1] |
| 445 | 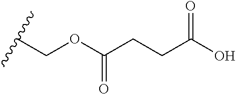 [SMILES: *COC(=O)CCC(=O)O] |
| 446 | 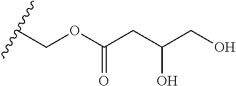 [SMILES: *COC(=O)CC(O)CO] |
| 447 | 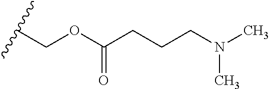 [SMILES: *COC(=O)CCCN(C)C] |
| 448 | CH₂OPO(OH)₂ |
| 449 | 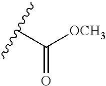 [SMILES: *C(=O)OC] |
| 450 | 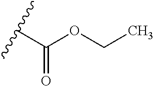 [SMILES: *C(=O)OCC] |
| 451 | 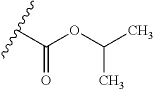 [SMILES: *C(=O)OC(C)C] |
| 452 | 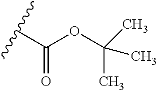 [SMILES: *C(=O)OC(C)(C)C] |
| 453 | 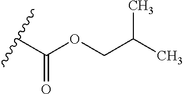 [SMILES: *C(=O)OCC(C)C] |
| 454 | 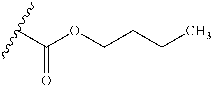 [SMILES: *C(=O)OCCCC] |
| 455 | 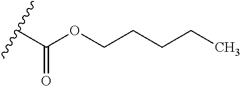 [SMILES: *C(=O)OCCCCC] |
| 456 | 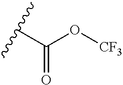 [SMILES: *C(=O)OC(F)(F)F] |
| 457 | 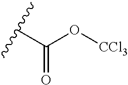 |
| 458 | 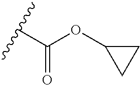 [SMILES: *C(=O)OC1CC1] |
| 459 | 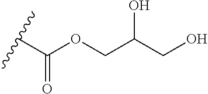 [SMILES: *C(=O)OCC(O)CO] |
| 460 | 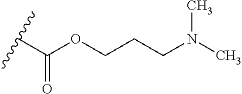 [SMILES: *C(=O)OCCCN(C)C] |
| 461 | 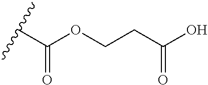 [SMILES: *C(=O)OCCC(=O)O] |
| 462 | 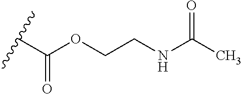 [SMILES: *C(=O)OCCNC(C)=O] |
| 463 | 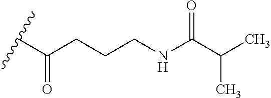 [SMILES: *C(=O)CCCNC(=O)C(C)C] |
| 464 | 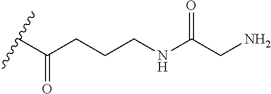 [SMILES: *C(=O)CCCNC(=O)CN] |
| 465 | 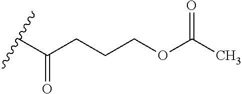 [SMILES: *C(=O)CCCOC(C)=O] |
| 466 | 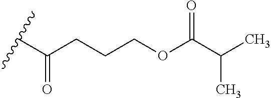 [SMILES: *C(=O)CCCOC(=O)C(C)C] |
| 467 | 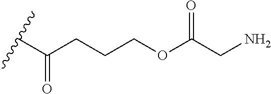 [SMILES: *C(=O)CCCOC(=O)CN] |
| 468 | 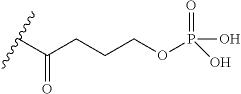 [SMILES: *C(=O)CCCOP(=O)(O)O] |
| 469 | 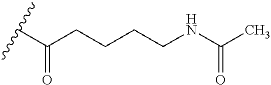 [SMILES: *C(=O)CCCCNC(C)=O] |
| 470 | 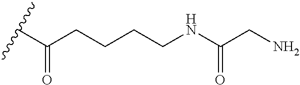 [SMILES: *C(=O)CCCCNC(=O)CN] |
| 471 | 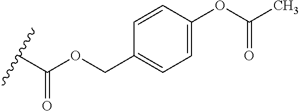 [SMILES: *C(=O)OCc1ccc(OC(C)=O)cc1] |
| 472 | 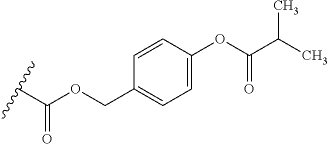 [SMILES: *C(=O)OCc1ccc(OC(=O)C(C)C)cc1] |
| 473 | 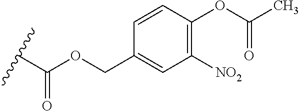 [SMILES: *C(=O)OCc1ccc(OC(C)=O)c([N+](=O)[O-])c1] |
| 474 | 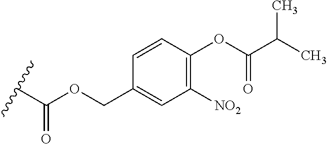 [SMILES: *C(=O)OCc1ccc(OC(=O)C(C)C)c([N+](=O)[O-])c1] |
| 475 | 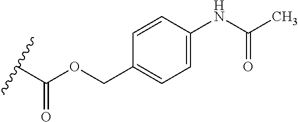 [SMILES: *C(=O)OCc1ccc(NC(C)=O)cc1] |
| 476 | 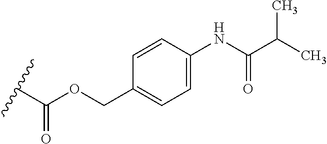 [SMILES: *C(=O)OCc1ccc(NC(=O)C(C)C)cc1] |
| 477 | 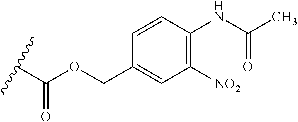 [SMILES: *C(=O)OCc1ccc(NC(C)=O)c([N+](=O)[O-])c1] |
| 478 | 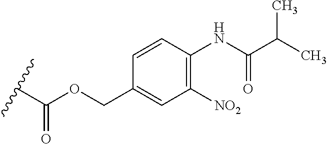 [SMILES: *C(=O)OCc1ccc(NC(=O)C(C)C)c([N+](=O)[O-])c1] |
| 479 | 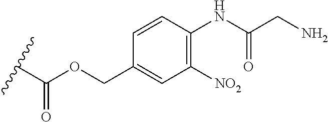 [SMILES: *C(=O)OCc1ccc(NC(=O)CN)c([N+](=O)[O-])c1] |
| 480 | 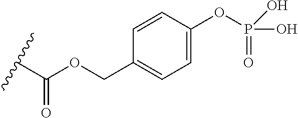 [SMILES: *C(=O)OCc1ccc(OP(=O)(O)O)cc1] |
| 481 | 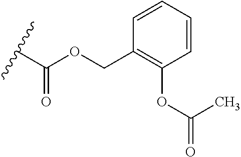 [SMILES: *C(=O)OCc1ccccc1OC(C)=O] |
| 482 | 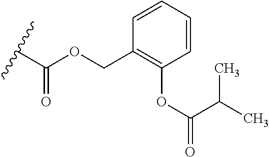 [SMILES: *C(=O)OCc1ccccc1OC(=O)C(C)C] |
| 483 | 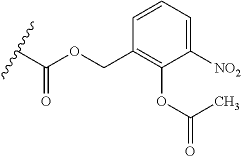 [SMILES: *C(=O)OCc1cccc([N+](=O)[O-])c1OC(C)=O] |
| 484 | 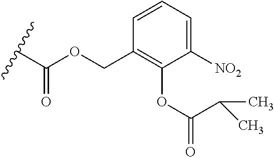 [SMILES: *C(=O)OCc1cccc([N+](=O)[O-])c1OC(=O)C(C)C] |
| 485 | 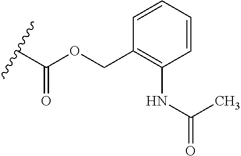 [SMILES: *C(=O)OCc1ccccc1NC(C)=O] |
| 486 | 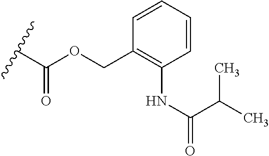 [SMILES: *C(=O)OCc1ccccc1NC(=O)C(C)C] |
| 487 | 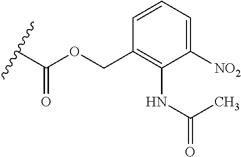 [SMILES: *C(=O)OCc1cccc([N+](=O)[O-])c1NC(C)=O] |
| 488 | 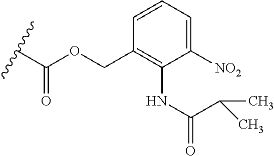 [SMILES: *C(=O)OCc1cccc([N+](=O)[O-])c1NC(=O)C(C)C] |
| 489 | 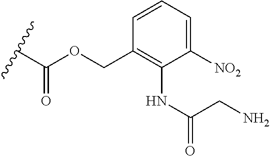 [SMILES: *C(=O)OCc1cccc([N+](=O)[O-])c1NC(=O)CN] |
| 490 | 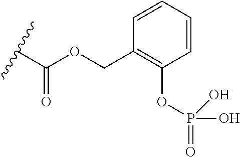 [SMILES: *C(=O)OCc1ccccc1OP(=O)(O)O] |
| 491 | 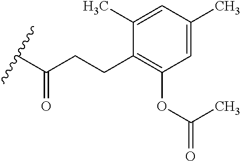 [SMILES: *C(=O)CCc1c(C)cc(C)cc1OC(C)=O] |
| 492 | 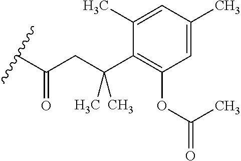 [SMILES: *C(=O)CC(C)(C)c1c(C)cc(C)cc1OC(C)=O] |
| 493 | 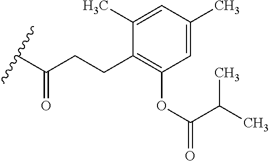 [SMILES: *C(=O)CCc1c(C)cc(C)cc1OC(=O)C(C)C] |
| 494 | 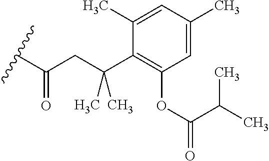 [SMILES: *C(=O)CC(C)(C)c1c(C)cc(C)cc1OC(=O)C(C)C] |
| 495 | 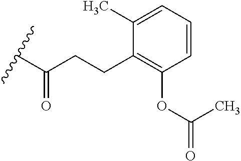 [SMILES: *C(=O)CCc1c(C)cccc1OC(C)=O] |
| 496 | 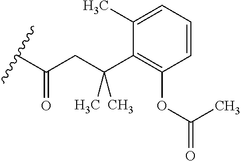 [SMILES: *C(=O)CC(C)(C)c1c(C)cccc1OC(C)=O] |
| 497 | 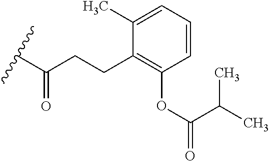 [SMILES: *C(=O)CCc1c(C)cccc1OC(=O)C(C)C] |
| 498 | 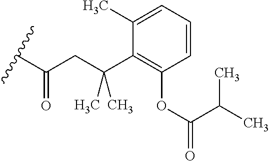 [SMILES: *C(=O)CC(C)(C)c1c(C)cccc1OC(=O)C(C)C] |
| 499 | 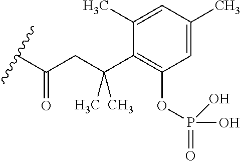 [SMILES: *C(=O)CC(C)(C)c1c(C)cc(C)cc1OP(=O)(O)O] |
| 500 | 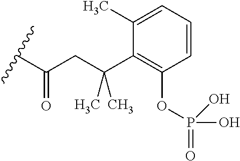 [SMILES: *C(=O)CC(C)(C)c1c(C)cccc1OP(=O)(O)O] |
| 501 | 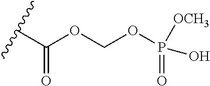 [SMILES: *C(=O)OCOP(=O)(O)OC] |
| 502 | 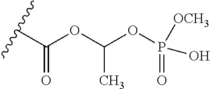 [SMILES: *C(=O)OC(C)OP(=O)(O)OC] |
| 503 | 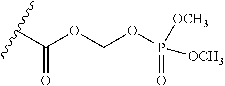 [SMILES: *C(=O)OCOP(=O)(OC)OC] |
| 504 | 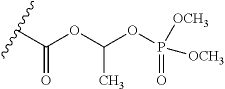 [SMILES: *C(=O)OC(C)OP(=O)(OC)OC] |
| 505 | 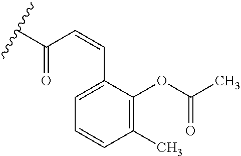 [SMILES: *C(=O)/C=C\c1cccc(C)c1OC(C)=O] |
| 506 | 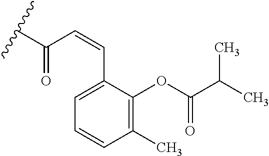 [SMILES: *C(=O)/C=C\c1cccc(C)c1OC(=O)C(C)C] |
| 507 | 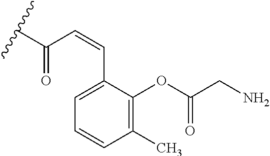 [SMILES: *C(=O)/C=C\c1cccc(C)c1OC(=O)CN] |
| 508 | 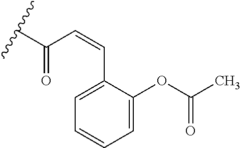 [SMILES: *C(=O)/C=C\c1ccccc1OC(C)=O] |
| 509 | 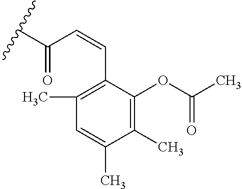 [SMILES: *C(=O)/C=C\c1c(C)cc(C)c(C)c1OC(C)=O] |
| 510 | 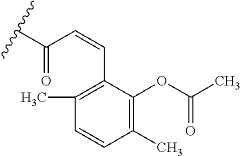 [SMILES: *C(=O)/C=C\c1c(C)ccc(C)c1OC(C)=O] |
| 511 | 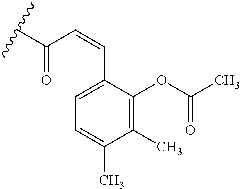 [SMILES: *C(=O)/C=C\c1ccc(C)c(C)c1OC(C)=O] |

### Compound 1: (2S)-2,6-diamino-N-(1-(benzo[id][1i,3]dioxol-5-yl)-1-oxopropan-2-yl)-N-methylhexanamide

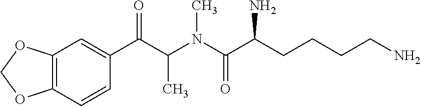 [SMILES: CC(C(=O)c1ccc2c(c1)OCO2)N(C)C(=O)[C@@H](N)CCCCN]

Compound 1 was prepared by the following procedure: Step 1: di-tert-butyl ((5S)-6-((1-(benzo[d] [1,3]dioxol-5-yl)-1-oxopropan-2-yl)(methyl)amino)-6-oxohexane-1,5-diyl)dicarbamate. To a solution of methylone hydrochloride (1.03 g) in 100 mL of CH₂Cl₂ at room temperature was added diisopropylethylamine (3.6 mL), HOBT (0.87 g), di-Boc-Lysine (1.7 g), EDC (0.9 mL) and DMAP (0.1 g). The reaction was sturred overnight at room temperature followed by the addition of 100 mL of CH₂Cl₂. The resulting solution was washed with 200 mL of 1 M HCl, 200 mL of aqueous saturated NaHCO₃ and 200 mL of aqueous saturated NaCl. The organic layer was dried over sodium sulfate, concentrated under reduced pressure, and purified by silica gel column chromatography using 30-50% EtOAc in hexane. The pure fractions were then combined and concentrated to afford the desired Boc protected intermediate as an off-white solid.

Step 2: (2S)-2,6-diamino-N-(1-(benzo[d][1,3]dioxol-5-yl)-1-oxopropan-2-yl)-N-methylhexanamide. To a CH₂Cl₂ (10 mL) solution of di-tert-butyl ((5S)-6-((1-(benzo[d][1,3]dioxol-5-yl)-1-oxopropan-2-yl)(methyl)amino)-6-oxohexane-1,5-diyl)dicarbamate from step 1, was added 10 mL of trifluoroacetic acid. The reaction was stirred 4 hours at room temperature, diluted with 10 mL of CH₂Cl₂ and brought to pH 1 with 20 mL of 1 M HCl. Layers were separated and 20% NaOH_{aq}. was added to the aqueous layer to bring the pH >10. This resulting basic aqueous layer was extracted twice with 20 mL of CH₂Cl₂. The combined organic layers were concentrated under reduced pressure to afford compound 1 as a solid.

### Compound 2: 2-amino-N-(1-(benzo[d][1,3]dioxol-5-yl)-1-oxopropan-2-yl)-N-methylacetamide

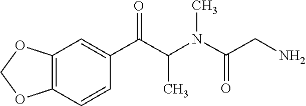 [SMILES: CC(C(=O)c1ccc2c(c1)OCO2)N(C)C(=O)CN]

Compound 2 was prepared by the following procedure: Step 1: tert-butyl (2-((1-(benzo[d][1,3]dioxol-5-yl)-1-oxopropan-2-yl)(methyl)amino)-2-oxoethyl)carbamate. To a solution of methylone hydrochloride (0.5 g) in 50 mL of CH₂Cl₂ at room temperature was added diisopropylethylamine (1.8 mL), Boc-Glycine (0.47 g), EDC (0.5 mL), DMAP (0.1 g) and HOBT (0.42 g). The reaction was stirred overnight at room temperature followed by the addition of 50 mL of CH₂Cl₂. The resulting solution was washed with 100 mL of 1 M HCl, 100 mL of aqueous saturated NaHCO₃ and 100 mL of aqueous saturated NaCl. The organic layer was concentrated under reduced pressure to afford an off-white solid. This crude product was carried to the next step without purification.

Step 2: 2-amino-N-(1-(benzo[d][1,3]dioxol-5-yl)-1-oxopropan-2-yl)-N-methylacetamide. To a CH₂Cl₂ (10 mL) solution of tert-butyl (2-((1-(benzo[d][1,3]dioxol-5-yl)-1-oxopropan-2-yl)(methyl)amino)-2-oxoethyl)carbamate from step 1, was added 10 mL of trifluoroacetic acid. The reaction was stirred 4 hours at room temperature, diluted with 30 mL of CH₂Cl₂ and brought to pH 1 with 20 mL of 1 M HCl. Layers were separated and 20% NaOH_{aq}. was added to the aqueous layer to bring the pH >10. This resulting basic aqueous layer was extracted twice with 20 mL of CH₂Cl₂. The combined organic layers were concentrated under reduced pressure to afford compound 2 as a solid.

### Compound 25: N-(1-(benzo[d][1,3]dioxol-5-yl)-1-oxopropan-2-yl)-N-methylacetamide

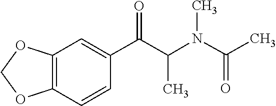 [SMILES: CC(=O)N(C)C(C)C(=O)c1ccc2c(c1)OCO2]

Compound 25 was prepared by the following procedure: To a solution of methylone hydrochloride (0.5 g) in 50 mL of CH₂Cl₂ was added diisopropylethylamine (0.9 mL). The solution was stirred 15 minutes at room temperature, cooled down to 0° C. and acetyl chloride (0.3 mL) was added. After 30 minutes at 0° C., the reaction was allowed to warm up to room temperature and stirred overnight. Volatiles were then removed under reduced pressure to afford a yellow solide that was dissolved in 150 mL of CH₂Cl₂. The resulting solution was washed twice with 100 mL aqueous saturated NaHCO₃ and 100 mL of aqueous saturated NaCl. The organic layer was concentrated under reduced pressure to yield compound 25 as a solid.

Compound 45: N-(1-(benzo[d][1,3]dioxol-5-yl)-1-oxopropan-2-yl)-2,2,2-trifluoro-N-methylacetamide.

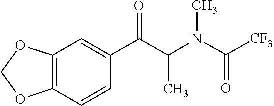 [SMILES: CC(C(=O)c1ccc2c(c1)OCO2)N(C)C(=O)C(F)(F)F]

Compound 45 was prepared by the following procedure: to a 25 mL round-bottomed flask under nitrogen was charged methylone hydrochloride (500 mg, 2.05 mmol, 1.0 eq) and DCM (4 mL, 8 vol) with DIPEA (1.09 mL, 6.25 mmol, 3 eq). After stirring for 10 mins a light brown solution formed. The solution was cooled to 0° C. and trifluoracetic anhydride (483 mg, 2.3 mmol, 1.12 eq) in DCM (1 mL, 2 vol) was charged dropwise, off gassing and a small exotherm from 4° C. to 10° C. was observed. After 30 mins of stirring at 0° C. to 10° C., HPLC monitoring indicated 66% product and 33% starting material. An additional charge of DIPEA (0.44 mL, 1.23 mmol eq) and trifluoracetic anhydride (237 mg, 0.55 eq) was made, and the reaction stirred overnight at ambient temperature. HPLC analysis the following day showed 95% product and no detectable starting material. The reaction was washed with water (5 mL×2), the DCM layer was dried (MgSO4) and concentrated to afford an orange solid. The solid was purified by column chromatography (10 g silica, 100% DCM) to give 372 mg of compound 45 as a solid.

Compound 50: 1-(((1-(benzo[d][1,3]dioxol-5-yl)-1-oxopropan-2-yl)(methyl)carbamoyl)oxy)ethyl isobutyrate.

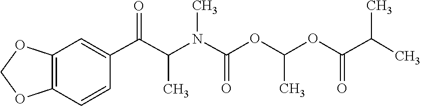 [SMILES: CC(OC(=O)C(C)C)OC(=O)N(C)C(C)C(=O)c1ccc2c(c1)OCO2]

Compound 50 was prepared by the following procedure: To a 0° C. suspension of methylone hydrochloride (1.35 g) in CH₂Cl₂ (10 mL) was added triethylamine (1.19 g in 2 mL of CH₂Cl₂). The resulting beige solution was stirred at 0° C. for 10 minute followed by the dropwise addition of 1-(((4-Nitrophenoxy)carbonyl)oxy)ethyl isobutyrate (2.0 g in 4 mL of CH₂Cl₂) over 5 minutes. The reaction mixture was stirred between −5 to 5° C. for lh then warmed up to RT (15-20° C.) and stirred over the weekend (˜66h) and an orange solution was obtained. 1M aqueous acetic acid (7 mL) was then added dropwise over 5 minutes below 25° C. and stirred for 5 minutes. The phases were separated and the organic layer was washed with 1M aqueous K₂CO₃ (3×7 mL) and then 20% aqueous brine (7 mL). The material was concentrated in vacuo at 30° C. then redissolved in ethyl acetate (10 mL). The organic was washed with 1M aqueous K₂CO₃ (2×7 mL) followed by 20% aqueous brine (7 mL) and then concentrated in vacuo at 40° C. The crude material was purified via silica gel column chromatography and eluted with 1-10% ethyl acetate in heptane. The clean fractions were concentrated in vacuo at 40° C. then stripped from TBME (3×20 mL) to afford compound 50.

Compound 71: methyl (1-(benzo[d][1,3]dioxol-5-yl)-1-oxopropan-2-yl)(methyl)carbamate.

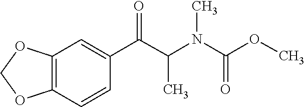 [SMILES: COC(=O)N(C)C(C)C(=O)c1ccc2c(c1)OCO2]

Compound 71 was prepared by the following procedure: to a 25 mL round-bottomed flask under nitrogen was charged methylone hydrochloride (500 mg, 2.05 mmol, 1.0 eq) with DCM (4 mL, 8 vol) and DIPEA (1.09, 6.25 mmol, 3 eq). The reaction was cooled to 0° C. and methyl chloroformate (257 mg, 2.7 mmol, 1.3 eq) in DCM (1 mL, 2 vol) was charged dropwise over 5 minutes to form a pale brown solution, an exotherm from 6° C. to 12° C. was observed. HPLC analysis showed the starting material had been consumed. The reaction was worked up by washing with water (5 mL×2) using a phase separator. The DCM concentrated to afford a clear oil. The oil was purified using column chromatography (10 g silica, 100% DCM) to afford 197 mg of compound 71 as a clear oil.

Compound 77: pentyl (1-(benzo[d][1,3]dioxol-5-yl)-1-oxopropan-2-yl)(methyl)carbamate.

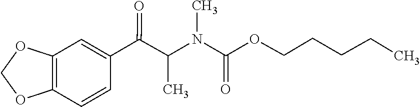 [SMILES: CCCCCOC(=O)N(C)C(C)C(=O)c1ccc2c(c1)OCO2]

Compound 77 was prepared by the following procedure: To a solution of methylone hydrochloride (0.5 g) in 50 mL of CH₂Cl₂ was added diisopropylethylamine (0.9 mL) and triethylamine (0.46 mL). The solution was stirred 15 minutes at room temperature, cooled down to 0° C. and amyl chloroformate (0.5 mL) was added dropwise. The reaction was allowed to warm up to room temperature and stirred for 90 minutes. Volatiles were removed under reduced pressure to afford an off-white solide that was then dissolved in 100 mL of CH₂Cl₂. The resulting solution was washed twice with 100 mL aqueous saturated NaHCO₃ and 100 mL of aqueous saturated NaCl. The organic layer was concentrated under reduced pressure to afford compound 77 as a solid.

### Example 2

### Assessment of Prodrug Effects and Stereoisomers on the PHARMACOKINETIC PROPERTIES AND EFFICACY OF METHYLONE

The pharmacokinetic properties of methylone after a single administration intravenously (IV), intraperitoneally (IP), or by oral gavage (PO) in male Sprague Dawley rats has been determined using a liquid chromatography tandem mass spectrometry (LC-MS/MS) method that has been established and validated for methylone in the rat. Rats (n=3 per group) received a single dose of methylone: 5 mg/kg IV, 15 mg/kg IP, or 15 mg/kg PO. Plasma was sampled at time points between 0.083-24 hours, methylone levels determined, and key parameters (e.g., Cₘₐₓ, Tₘₐₓ, T_{1/2}, and AUC) were analyzed from the data. Results are shown in TABLE 8.

TABLE 8
Selected pharmacokinetic parameters of methylone following a single
IV, PO and IP administration to male Sprague-Dawley rats

| PK | Methylone | | |
|---|---|---|---|
| Parameters | Group 1 (IV) | Group 2 (PO) | Group 3 (IP) |
| Cₘₐₓ (ng/mL) | N/A | 678 ± 243 | 4230 ± 514 |
| Tₘₐₓ (h) | N/A | 1.33 ± 0.577 | 0.250 ± 0.000 |
| T_{1/2} (h) | 0.362 ± 0.024 | 3.23 ± 1.99 | 0.800 ± 0.169 |
| C₀ (ng/mL) | 3018 ± 666 | N/A | N/A |
| Vdₛₛ (L/kg) | 1.98 ± 0.230 | N/A | N/A |
| Cl (mL/min/kg) | 68.3 ± 5.40 | N/A | N/A |
| AUC₀₋ₗₐₛₜ | 1200 ± 88.2 | 2765 ± 657 | 4305 ± 1179 |
| (ng · h/mL) | | | |
| MRT₀₋ₗₐₛₜ (h) | 0.439 ± 0.026 | 2.99 ± 0.467 | 1.07 ± 0.211 |

To investigate whether the prodrug extends the half-life or alters other basic pharmacokinetic properties of methylone (e.g., Cₘₐₓ or Tₘₐₓ), rats are treated with each prodrug IV, IP, or PO. For each compound, three groups of rats are treated as follows: For group 1, a single dose of methylone is administered to 3 male Sprague-Dawley rats by IV bolus at 5 mg/kg. For group 2, a single dose of methylone is administered to 3 male Sprague-Dawley rats by oral gavage (PO) at 15 mg/kg. For group 3, a single dose of methylone is administered to 3 male Sprague-Dawley rats by IP at 15 mg/kg. For all groups, blood samples are collected from each animal at 0.083, 0.25, 0.5, 1, 2, 4, 8, 12, and 24 hours post-dose for the determination of plasma concentrations. The plasma concentrations are quantified by liquid chromatography tandem mass spectrometry (LC-MS/MS). The bioanalytical assay has been established and validated, providing a lower limit of quantification (LLOQ) of 1 ng/mL and an upper limit of quantification (ULOQ) of 3000 ng/mL for methylone. The plasma concentration-time data are analyzed using Phoenix WinNonlin (version 8.3) to characterize the PK properties of the analyte. The non-compartmental analysis model and the linear/log trapezoidal method are applied to calculating the PK parameters.

### Metabolic Stability

Stability in whole blood

Test compounds (prodrugs, concentration 2 μM) are spiked into pre-warmed fresh rat or human whole blood (EDTA K3) and incubated at 37° C. for up to 2 hours in duplicate. The disappearance of test compound and accumulation of methylone is monitored at 0, 10, 30, 60, and 120 minutes.

Stability in buffer or simulated gastric fluid (SGF)

Test compounds (prodrugs, concentration 2 μM) are spiked into buffer (citrate pH 4.5, citrate or phosphate pH 6.8) or into SGF and incubated at 37° C. for 2 hours in triplicate. The disappearance of test compound and accumulation of methylone is monitored at 0, 10, 30, 60, and 120 minutes.

### Hepatocyte Stability

Test compounds (prodrugs, concentrations 0.1-20 mg/mL) are incubated with human or rat hepatocytes at 37° C. Reactions are terminated at the appropriate time points (including the time points: 0, 10, 30, 60, 90 and 120 minutes) by adding cold acetonitrile containing internal standards (IS). Following centrifugation, the supernatant is analyzed by LC-MS/MS. The disappearance of test compound and accumulation of methylone is monitored over the time period.

Stock solutions are prepared in dimethyl sulfoxide (DMSO) and stored in a freezer at −20° C. Further dilutions are prepared in an appropriate solvent on the day of the experiment. Positive control solutions are prepared in the same manner. Further dilutions are prepared on the day of the experiment. The organic content is <1.0% in the final incubation.

The quenching solution is prepared with acetonitrile containing tolbutamide/labetalol (internal standard) for incubation samples. The detailed concentrations are recorded. The quenching solution is stored at room temperature and kept on ice prior to use.

Cryopreserved hepatocytes are thawed and isolated using the cryopreserved hepatocyte thawing medium. Viability of the hepatocytes is determined using the Trypan Blue exclusion method and viable cells should be >70%. Cell suspensions are prepared with Williams' E Medium to an appropriate concentration.

Incubations are conducted in 96-well plate. The test article is incubated with hepatocytes suspension in triplicate at a cell density of 0.5×10⁶ cells/mL. Then the sample plates are incubated in an incubator at 37° C. with 5% CO2 and 95% relative humidity with oscillation at 150 rpm on a plate shaker. At each specified time point, the incubation is terminated by the addition of 3 volumes of cold quench solution.

Positive controls, 7-ethoxycoumarin and 7-hydroxycoumarin, are incubated in parallel at 3 μM. Medium control samples are included in the absence of cells. The total organic concentration in the assay mixture is <1%.

All sample plates are placed on a plate shaker at 500 rpm for 15 minutes and centrifuged at 3,220×g for 20 minutes. The supernatants are diluted with water/organic mixture if necessary at an appropriate ratio for LC/MS/MS analysis.

Concentrations of test compound and control compounds in the samples are analyzed by using a liquid chromatography-tandem triple quadrupole mass spectrometry (LC-MS/MS) method. Plotting of the chromatograms and peak area integrations are carried out.

Concentrations of test articles, methylone, and control compounds in the samples are determined using a semi-quantitative method, i.e., using a peak area ratio of analyte to internal standard. No calibration curve and QC are applied, and the peak area ratios of analyte/internal standard are used as concentrations in samples.

The in vitro elimination constant of compounds, kₑ, is calculated from a log linear plot of concentration or analyte/internal standard peak area ratios versus time and the half-life in minutes (t_{1/2}) is determined using the equation:

t_{1/2}(min)=0.693/kₑ

The estimation of the in vitro hepatic intrinsic clearance values (CLint) is calculated from substrate disappearance rate in hepatocyte incubations as follows:

$${CL}_{int ( hep )} = 0.693 / half - life / million cells$$

$${CL}_{int ( liver )} = {CL}_{int ( hep )} \times Hepatocellularity \times Liver Scaling Factor$$

The parameters used in the equations are summarized in TABLE 9 below.

TABLE 9

| | Liver Weight | Hepatocellularity |
|---|---|---|
| Species | (g/kg Body Weight) | (million cells/g liver) |
| Mouse | 88 | 135 |
| Rat | 40 | 117 |
| Minipig | 17 | 124 |
| Dog | 32 | 215 |
| Human | 20 | 139 |

Behavioral pharmacology studies to assess efficacy of the prodrugs and enantiomers

### Antidepressant Efficacy in The Forced Swim Test (FST)

The forced swim test (FST) is a classic model to assess the antidepressant-like activity of compounds that has been in use for over 40 years (Porsolt et al. (1977) Nature 266:730-732; Detke et al. (1995) Psychopharmacology 121:66-72). All classes of antidepressants, including selective serotonin reuptake inhibitors, noradrenergic reuptake inhibitors, tricyclics, and more recent rapidly acting antidepressants like ketamine, psilocybin, or MDMA have all been shown to reduce immobility in the FST. Methylone has a robust, dose-dependent antidepressant-like effect in the rat forced swim test (FST). A single dose of 5 mg/kg methylone reduces immobility by approximately 50% compared to vehicle-treated controls, whereas a 15 mg/kg dose reduces immobility by nearly 100%. Accompanying changes in climbing and/or swimming behavior reflect noradrenergic and serotonergic activities of methylone, respectively.

All FST studies are performed and scored by an experimenter blind to treatment group and according to standard protocols. Briefly, rats are placed in a circular plexiglass container filled with water. Water temperature is maintained at 22-25° C. and changed for every animal. Day 1 (Training) consists of a 15 min acclimation trial, and Day 2 (Testing, 24 h later) consists of the 5 min test. A time sampling procedure is employed where animals are observed every 5 seconds for the duration of the test session (60 counts or 5 minutes) and scored for immobility (defined as the failure to struggle), swimming (defined as a circular movement around the tank), or climbing (defined as an upwards escape behavior). Data are expressed as the percent of the testing session (e.g., the number of immobility counts divided by 60). A p-value less than 0.05 indicates statistical significance after typical statistical analyses (e.g., unpaired t-test or ANOVA).

To determine that the prodrug and/or enantiomer has an antidepressant-like effect and to compare it to methylone, rats are treated with each compound 30 min prior to testing. Additional tests are run 24, 72, 168 hours post-dose or longer. Effects on Fear Extinction in a Model of Post-Traumatic Stress Disorder (PTSD)

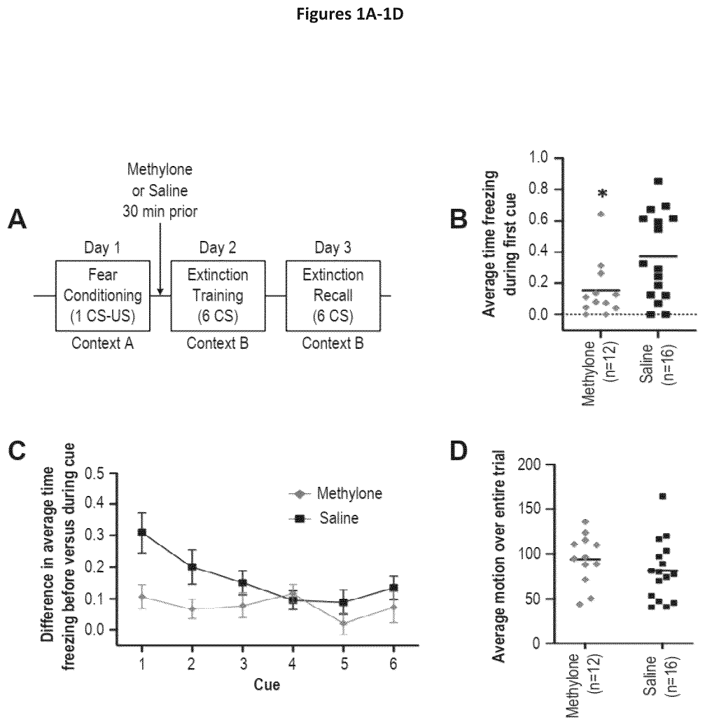

Methylone (30 mg/kg, IP) significantly improves fear extinction recall in a mouse model of PTSD (FIG. 1). Deficient fear extinction memory is a feature of PTSD in patients (Wicking et al. (2016) Neurobiology of Learning and Memory 136:116). SSRI antidepressants, similar to the two approved for the treatment of PTSD (i.e., paroxetine and sertraline), prevent fear memory generalization and enhance extinction (Pedraza et al. (2019) Transl Psychiatry 9:53). The enhancement of fear extinction might also underlie the beneficial effect of MDMA as a PTSD treatment (Feduccia & Mithoefer (2018) Progress in Neuro-Psychopharmacology & Biological Psychiatry 84(Part A), 221-228).

Effective PTSD treatments facilitate the disassociation between a traumatic memory and the patient's fear response, making cues for the traumatic memory evoke less of a fear response. This is modeled in the mouse fear extinction paradigm which takes place over 3 days. On day 1 (fear conditioning), mice are trained to acquire a “traumatic memory,” namely associating the conditioned stimulus (CS, tone) to the unconditioned stimulus (US, foot shock). On day 2 (extinction training), they are trained to forget the traumatic memory association by presenting the CS 6 times (with no US) in a novel environment. On day 3 (extinction recall), the mice are “asked” if that tone (CS) still elicits a fearful response, as measured by the time spent freezing when the tone is presented. Less time freezing means better extinction recall. Drugs that improve extinction recall reduce freezing time on day 3, and, therefore, show potential as a PTSD treatment.

Work with MDMA shows that after fear conditioning, administering MDMA (7.5 mg/kg) 30 minutes prior to extinction training enhances extinction recall measured as 35% reduced freezing compared to saline injected controls (Young et al. (2015) Transl Psychiatry 5:e634). Using a similar experimental design, recent results show that methylone (30 mg/kg) significantly enhances fear extinction recall (FIG. 1) by nearly 60% compared to saline controls (FIG. 1B). Using these methods, prodrugs and/or enantiomers are administered to mice and tested for efficacy in the fear extinction model to evaluate their efficacy to treat PTSD and other memory disorders. Anxiolytic Effects in Other Behavioral Anxiety Models

Additional behavioral testing including the elevated plus maze (EPM) and thigmotaxis in the open field test (OFT) are used to assess the anxiolytic effects of methylone and its prodrugs in mice or rats. Methylone (5, 10, 20 mg/kg, SC) has been shown to reduce thigmotaxis (time spent hugging the perimeter of the open field) in rats (Stefkovi et al. (2017) Front Psychiatry 8:232.), consistent with an anxiolytic effect. These models are described in greater detail below. Prodrugs are screened in these behaviors to the anxiolytic efficacy of each compound.

Methylone reduces time spent in the center vs the periphery in the OFT, consistent with an anxiolytic-like response. Methylone is also a stimulant that increases locomotor activity in this test. Compounds are screened for their effects on both parameters. Briefly, rodents are assessed in a 30 minute OFT using an automated activity monitoring system. Rodents are acclimated to the room 30 minutes before the start of testing. The following parameters are captured: Horizontal distance travelled, overall ambulatory time, and ambulatory counts. Vertical activity (time and counts), Time in the Center vs. Periphery data are reported in 5-minute bins as well as total time.

The EPM is a classic anxiety model that also capitalizes on a rodent's dislike for open spaces. The effects of prodrug compounds and methylone are tested in this model. Briefly, rodents are acclimated to the anteroom at least 30 minutes before the start of the experiment. Testing is performed in dim light (40 lux). The elevated plus maze consists of two open and two closed arms (arm length: 30 cm; width: 5 cm). Open arms have a small 1 cm edge and the closed arms are bordered by a 15 cm wall. At the beginning of the task, rodents are placed in the center of the elevated plus maze facing an open arm and are videotracked while exploring the maze for 5 minutes. The time spent in the open and closed arms are measured and analyzed. More time in the open arm vs. the closed arm is consistent with an anxiolytic effect.

### Example 3

### Synthesis of Methylone

Methylone HCl was prepared in three stages starting from 3,4-methylenedioxypropiophenone (MDP), as shown in Scheme 18 below.

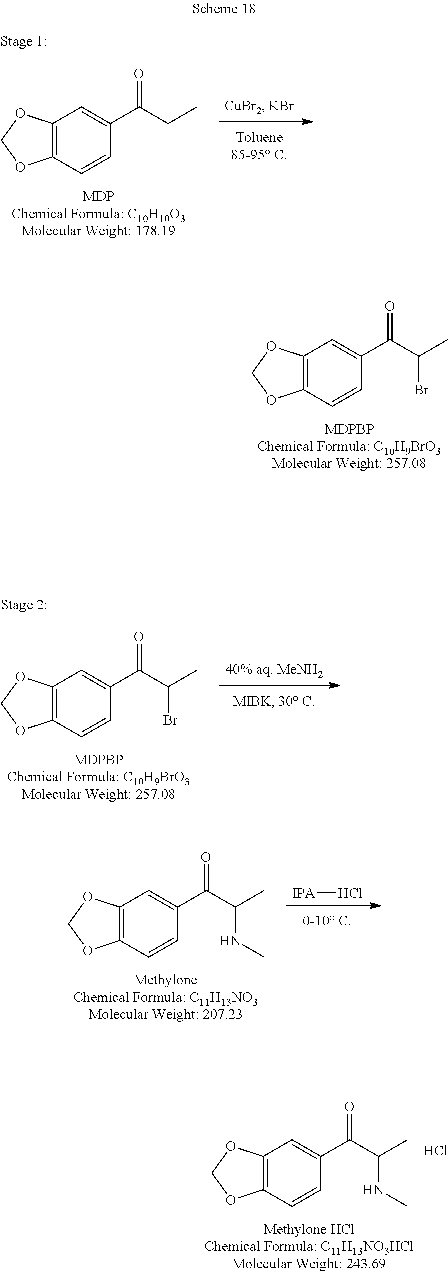

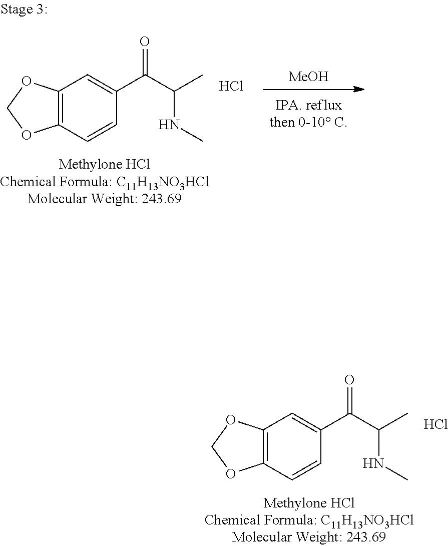

The chemical formula for methylone HCl is C₁₁H₁₃NO₃ HCl; molecular weight 243.7 g/mol (HCl), 207.2 g/mol (free base). The chemical structure of methylone HCl is

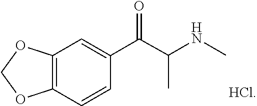

### Stage 1: 3,4-Methylenedioxypropiophenone (MDP) to 2-Bromo-3′,4′-(methylenedioxy)propiophenone (MDPBP)

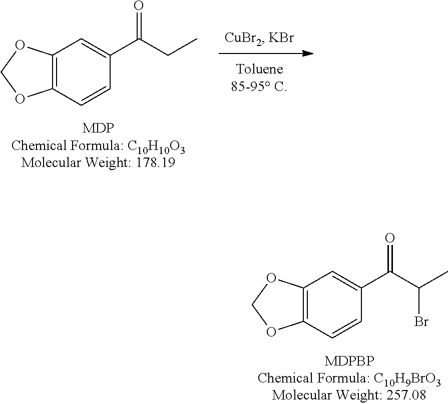

3,4-methylenedioxypropiophenone (MDP) was reacted with copper (II) bromide (CuBr2) and potassium bromide (KBr) in toluene. The resulting suspension reaction was heated to 85-95° C. for 24 hours. After completion, the reaction was cooled to 22-24° C. and filtered through celite to remove insoluble copper salts. The filtrate was washed with 2.8% ammonium hydroxide up to 5 times to remove soluble copper salts. The organic layer was distilled under reduced pressure to remove toluene, leaving a brown solid.

### Stage 2: 2-Bromo-3′,4′-(methylenedioxy)propiophenone (MDPBP) to Methylone HCl

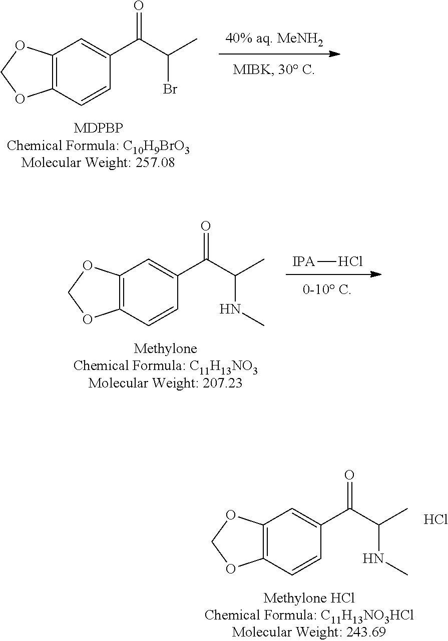

To a solution of 2-bromo-3′,4′-(methylenedioxy)propiophenone (MDPBP) in MIBK was charged 40% aq. Methylamine solution at 22±2° C. over 45±15 minutes. The reaction mixture was stirred at 30° C. for 4 hours. After completion, the reaction was quenched with 20% aq. sodium hydroxide (NaOH), while maintaining the temperature at 22±2° C. The layers were separated, and the organic layer was washed three times with water. The organic layer was cooled to 0-10° C. and 0.5-0.6N HCl in isopropyl alcohol (IPA) was added slowly, maintaining the temperature below 10° C. The resultant solution was stirred at 0-10° C. for 2 hours, and then filtered. The solid was washed with IPA, then sampled and its purity analyzed by HPLC (IPC2). If sample purity is >98% with no impurity >0.5% with a white to off-white appearance, the material was dried. If in process specifications were not met stage 3 purification was performed.

### Stage 3: Methylone HCl purification

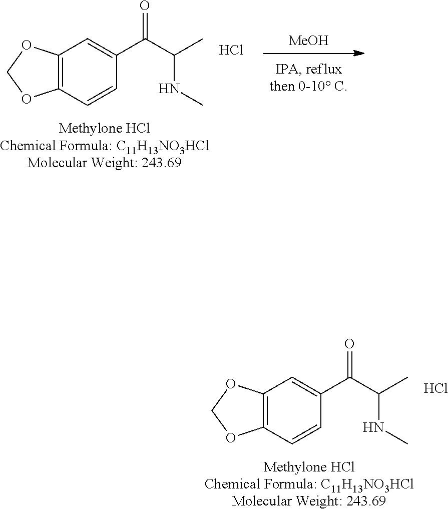

If methylone HCl does not meet in process specifications an additional purification step is required. A mixture of methylone HCl in methanol and isopropanol is heated to reflux (65° C.). The resultant solution stirred at reflux for 1 hour. The slurry is cooled to 0-10° C. and held at that temperature for 2 hours. The solids are filtered and washed with isopropanol, then dried under reduced pressure at 60° C. The resultant solid is sampled, and its purity analyzed by HPLC and moisture content analyzed by loss on drying (LOD).

In-process control (IPC) checks were conducted at various stages of the reaction, including verification of hold times, pH adjustments, etc. IPC testing for purity was used at each major step (stage) of the synthesis to assure sufficient quality of the intermediate product prior to proceeding to the next synthetic step. TABLE 10 presents the results of two lots prepared according to this Example, as well as the results for three (“REFERENCE”) lots prepared according to standard methods. The lots prepared according to this Example have improved purity and greater than an 8-fold increase in yield relative to the REFERENCE lots.

TABLE 10
Batch Analysis for Drug Substance Lots

| Test | Method | Results | | Results | | Results | | Results | | Results | |
|---|---|---|---|---|---|---|---|---|---|---|---|
| Lot | | REFERENCE | | REFERENCE | | REFERENCE | | THIS | | THIS | |
| | | | | | | | | EXAMPLE | | EXAMPLE | |
| Lot Size | | 50 | g | 50 | g | 50 | g | 650 | g | 650 | g |
| Appearance | Visual | Conforms | | Conforms | | Conforms | | Conforms | | Conforms | |
| Identity | USP<197>, | Conforms | | Conforms | | Conforms | | Conforms | | Conforms | |
| | FTIR | | | | | | | | | | |
| Loss on | USP<731> | 1.94% | | 1.58% | | 1.73% | | 0.1% | | 0.2% | |
| Drying | | | | | | | | | | | |
| (LOD) | | | | | | | | | | | |
| Water | USP<921>, | 0.02% | | 0.02% | | 0.03% | | 0.1% | | 0.2% | |
| Content | KF | | | | | | | | | | |
| Chloride | USP<221>, | 14.6% | | 14.5% | | 14.6% | | 14.4% | | 14.5% | |
| Content | titration | | | | | | | | | | |
| Purity | HPLC | 99.95% | wt | 99.95% | wt | 99.96% | wt | 100% | wt | 100% | wt |

| | | | | | | |
|---|---|---|---|---|---|---|
| Related | HPLC | 2,3- | 2,3- | 2,3- | 2,3- | 2,3- |
| Substances | | Methylone: | Methylone: | Methylone: | Methylone: | Methylone: |
| | | 0.02% | 0.02% | 0.02% | ND | ND |
| | | MDP: ND | MDP: ND | MDP: ND | MDP: ND | MDP: ND |
| | | MDPBP: | MDPBP: | MDPBP: | MDPBP: | MDPBP: |
| | | ND | ND | ND | ND | ND |
| | | Any | Any | Any | Any | Any |
| | | Unspecified | Unspecified | Unspecified | Unspecified | Unspecified |
| | | Impurity: | Impurity: | Impurity: | Impurity: | Impurity: |
| | | 0.03% | 0.03% | 0.02% | ND | ND |
| | | Total | Total | Total | Total | Total |
| | | Impurities: | Impurities: | Impurities: | Impurities: | Impurities: |
| | | 0.05% | 0.05% | 0.04% | ND | ND |

NMT = not more than; ND = not detected.

Potential sources of impurities in the methylone synthesized may include residual starting material, potential process impurities, and degradation products. These may include:

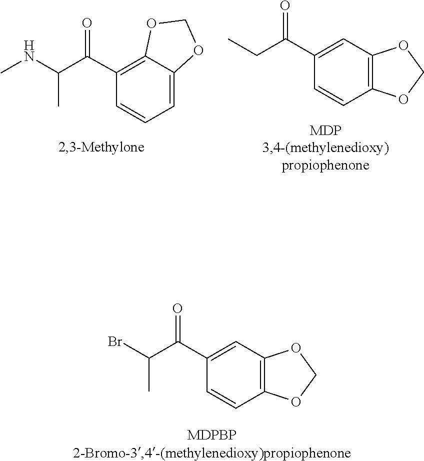

The following is a description of the HPLC method for use in determination of the assay and related substances.

TABLE 11
Chromatographic Conditions

| Column | Waters Symmetry C18 4.6 × 150 mm, 5μ | |
|---|---|---|
| Flow Rate | 1.0 | mL/min |
| Mode | Gradient | |
| Injection Volume | 10 | μL |
| Column Temperature | 25° C. ± 2° | |
| Detector | 254 | nm |
| Run Time | 32 | min |

TABLE 12
Gradient

| Time (min) | Flow (ml/min) | Mobile Phase A (%) | Mobile Phase B (%) |
|---|---|---|---|
| 0.0 | 1.0 | 80 | 20 |
| 25.0 | 1.0 | 20 | 80 |
| 27.0 | 1.0 | 80 | 20 |
| 32.0 | 1.0 | 80 | 20 |

### Solution Preparations:

### Mobile Phase A—(MPA)

Add 1000 mL water into a HPLC bottle. Add 200 μL of TEA (Triethylamine) and mix well.

### Mobile Phase B—(MPB)

Add 1000 mL of ACN into a HPLC bottle. Add 200 μL of TEA (Triethylamine) and mix well.

### Diluent

### Methanol

TABLE 13
Expected Retention Times:

| | Retention Time | Relative Retention |
|---|---|---|
| Compound | (minutes) | Time (RRT) |
| Methylone | 7.9 | 1.00 |
| 2,3 Methylone | 8.5 | 1.08 |
| MDP | 13.4 | 1.70 |
| MDPB | 16.8 | 2.13 |

The data in TABLE 14 compares methylone synthesized as described in this Example with “Reference” material commercially available from Cayman chemicals. The methylone synthesized as described in this Example has fewer impurities and at a lower level than the Reference material, which has an unspecified impurity greater than the qualification threshold for an impurity according to the ICH Q3A and ICH Q3B guidelines.

TABLE 14

| Test | Method | Present Material | Reference Material |
|---|---|---|---|
| Assay | HPLC | 99.96% | 99.65% |
| Related | HPLC | 2,3- | 2,3- |
| Substances | | Methylone: 0.02% | Methylone: 0.13% |
| | | Any Unspecified | Any Unspecified |
| | | Impurity: 0.02% | Impurity: 0.23% |
| | | Total: | Total: |
| | | 0.04% | 0.36% |

As MDPBP is electrophilic, and therefore potentially mutagenic, an in vitro Ames test was performed as described in Table 8. 2,3-Methylone and MDP are not electrophilic and therefore only in silico quantitative/qualitative structure activity relationship (QSAR) (Cayley et al. (2023) Regulatory Toxicology and Pharmacology 144:105490) was performed and found to be negative. Therefore, one aspect of the invention is the production of methylone which is not mutagenic and has no mutagenic impurities.

### Example 4

### Alternative Synthesis of Methylone

Methylone HCl was prepared starting from 3,4-methylenedioxypropiophenone (MDP) using an alternative synthesis, as described below.

### Stage 1:

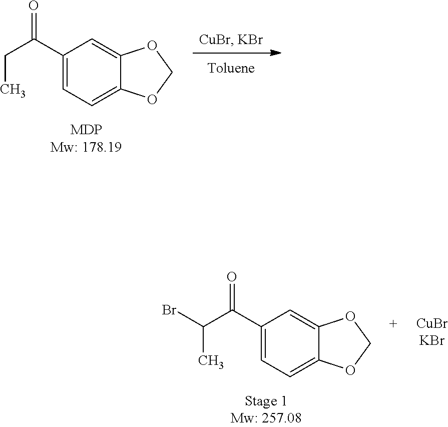

3,4-methylenedioxypropiophenone (MDP) in toluene (15 mL/g was reacted with copper (II) bromide (CuBr2) (2.9 g/g, 2.3 eq) and potassium bromide (KBr) (0.13 g/g, 0.2 eq), by heating to 85-95° C. and stirring for at least 24 hours. After completion, the reaction was cooled to 15-25° C. and filtered through celite 545 (0.79 g/g). The filtrate was washed twice with toluene and added to a clean vessel to which ammonia (1 mL/g) in water (9 mL/g) was then added and stirred for at least 5 minutes. The phases were allowed to separate, and the bottom aqueous layer was removed. Solids were observed on the interface of the two layers—these were retained in the organic layer. Twice more, ammonia (1 mL/g) in water (9 mL/g) was added and stirred for at least 5 minutes to allow phases to separate and the bottom aqueous layer was removed. Solids were observed on the interface of the two layers—these were retained in the organic layer. The aqueous layers were pooled and added back into the vessel. Toluene was added and stirred for at least 5 minutes and the layers were separated. The organic layers from this step and the previous step were combined into the vessel. 300 mL of THF was added to the organic layer on a 55g scale to help dissolve the solids. Saturated aqueous brine (10 mL/g) was added and stirred for at least 5 minutes to allow the phases to separate and the bottom aqueous layer was removed. The organic phase was dried over MgSO₄ and then filtered and washed with toluene (3 mL/g). The filtrate was evaporated to dryness (max temp 50° C.).

### Stage 2:

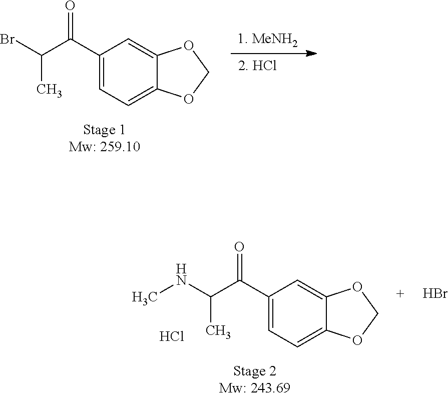

The product of the previous stage was dissolved in tetrahydrofuran (26 mL/g) and was added dropwise over 1 hour to stirring methylamine 40 wt % in water (1.67 mL/g, 5 eq) at 15-25° C. and then stirred for 3 hours. Then saturated brine (5 mL/g) at 15-25° C. and sodium hydroxide (0.15 g/g, 1 eq) at 15-25° C. were added and stirred for at least five minutes. The layers were separated and the bottom aqueous layer was removed from the vessel. Charge with water (2.5 mL/g) at 15-25° C. Concentrated sulfuric acid (1 mL/g) at 15-25° C. was added dropwise over about an hour while stirring, until pH 1 was reached. Charge n-heptane (5 mL/g) and stirred for at least 5 minutes and then the layers were separated. Tert-butyl methyl ether (TBME; 5 mL/g) was added and then stirred for at least 5 minutes. The layers were separated, and the bottom aqueous layer was added back into the vessel. This step was repeated two more times. Tert-butyl methyl ether (10 mL/g) was added and sodium hydroxide (1.6 g/g) in water (10 mL/g) at 15-25° C. was added dropwise over about an hour while stirring, until pH 12 was reached. Then saturated brine (5 mL/g) at 15-25° C. was added and stirred for at least five minutes. The layers were separated, and the bottom aqueous layer was added back into the vessel. Tert-butyl methyl ether (5 mL/g) was then added and stirred for at least 5 minutes. The layers were separated, and the bottom aqueous layer was added back into the vessel. Tert-butyl methyl ether (5 mL/g) was then added and stirred for at least 5 minutes, after which the layers were separated. The organic layers from the previous steps were pooled and added into a clean vessel and then cooled to 0-10° C. Hydrochloric acid (5-6 M) in 2-propanol (1 mL/g) at 0-10° C. was added dropwise over about 15 minutes. The resultant solution was stirred at 0-10° C. for at least one hour, and then filtered. The filtrate was washed with tert-butyl methyl ether (5 mL/g) and dried in vacuo at 45° C.

### Purification of Final Product

The final product was performed by slurrying the methylone hydrochloride in isopropyl alcohol at 60° C., cooling to 5-10° C. followed by filtration and drying. With a final purity of 99.1%.

### Example 5

### Purification and Separation of Methylone Enantiomers

A solid of a mixture of methylone enantiomers was dissolved in 60% Hexane/40% EtOH/0.1% diethylamine (DEA) to a concentration of 2 mg/mL. 4.2 mg (2.1 mL of starting material solution) was injected and loaded onto a CHIRALPAK IA® (10×250 mm, 5 micron) HPLC preparative column using 70% Hexane/30% EtOH/0.1% DEA as an eluent at a flow rate of 6.0 mL/min and monitoring at 228 nm. A volume (˜221 μL/injection=˜2 times molar equivalent of the expected DEA in each fraction) of 2.0 N HCl was pre-added to each fraction container. The first fraction (F1) was collected starting at inflection of the UV readout (˜7 mins) for 4.4 mL and then switched to the second fraction (F2). F2 was collected until the local UV minimum and then switched to the third fraction (F3). F3 was collected for 7.5 mL and then switched to the fourth fraction (F4). F4 was collected until 1 minute after UV reached baseline. Fraction containers were immediately transferred to a rotory evaporator (Rotation speed: 20-120 rpm; Bath temperature: 35° C.) after the final injection, and the eluent was removed via vacuum distillation until the material was under full vacuum. The resulting material was a white solid powder and was reconstituted in 100% EtOH and filtered via syringe through a 0.45 m filter into a 40 mL vial. At this point, the majority of the material is DEA-HCl salt.

To remove the DEA-HCl salt, the material was dissolved in 20% MeOH/80% H₂O/0.05% Trifluoroacetic acid (TFA) to a concentration of up to 20 mg/mL. TFA was chosen as the acidic modifier due to its relatively low boiling point (72.4° C.) close to that of the mobile phase solvent ethanol (78.4° C.).Up to 500 mg (limited by size of injection loop) was injected and loaded onto a Phenomenex Luna 10 m PREP C18(3), 250×50 mm HPLC preparative column using 20% MeOH/80% H₂O/0.05% TFA as an eluent at a flow rate of 100.0 mL/min and monitoring at 228 nm. A single fraction of the entire peak eluting at ˜11 minutes was collected. The single fraction container was immediately transferred to a rotory evaporator (Rotation speed: 20-120 rpm; Bath temperature: 60° C.) after the final injection, and the eluent was removed via vacuum distillation until the material was under full vacuum. The resulting material was an amber oil and was reconstituted in a minimal amount of 100% EtOH and filtered via syringe through a 0.45 m filter directly into the finished product container.

The container was adapted (via appropriate fittings) to the rotary evaporator, which was set up for processing under an inert atmosphere. There was a vacuum trap, in a Dewar flask, cooled by a dry ice/acetone bath in line on the vacuum tube. The solvent was carefully removed via vacuum distillation until the material was under full vacuum. The container was removed from the vacuum, sampled, and capped under an inert atmosphere. The final mass was recorded after sampling. Removal of DEA was confirmed via refractive index analysis.

The typical purity of the earlier-eluting (R)-methylone enantiomer was −99% ep and lost little of its enantiomeric purity during post-purification processing (˜98% ep). The typical purity of the later-eluting (S)-methylone enantiomer was −99% ep and lost little of its enantiomeric purity during post-purification processing (˜98% ep).

### Example 6

Competition Radioligand Binding Study of Methylone Enantiomers Indicates that (S)-Methylone has Antidepressant and Anxiolytic Benefit with Fewer Cardiovascular Side Effects.

Methylone acts on three monoamine transporters, the serotonin transporter (SERT), the dopamine transporter (DAT) and the norepinephrine transporter (NET). Racemic methylone binds to SERT, DAT, and NET, where it inhibits the reuptake and facilitates the release of the neurotransmitters serotonin (5HT), dopamine (DA), and norepinephrine (NE) through these transporters. The antidepressant-like and anxiolytic effects of racemic methylone are likely linked to its action at the SERT, whereas cardiovascular side effects may be related to actions at the NET. Therefore, this study was undertaken to examine whether either of the two methylone enantiomers showed preferential binding to SERT and/or less potent binding to NET in order to identify a compound that has the antidepressant-like and anxiolytic effects of racemic methylone with less of the cardiovascular side effects.

### Methods

Rat brain (Sprague Dawley, 200-250 g) synaptosomes were isolated from midbrain and hindbrain for serotonin transporter assays, striatum for dopamine transporter assays, and hippocampus plus overlying occipital cortex for norepinephrine transporter assays. Radioligands used were: [³H]Citalopram 81.4 Ci/mmol (PerkinElmer NET1039250UC; Lot No. 2960876). [3H]WIN35428 82.8 Ci/mmol (PerkinElmer NET1033250UC; Lot No. 2891473). [³H]Nisoxetine 79.2 Ci/mmol (PerkinElmer NET1084250UC; Lot No. 2970324). Non-specific compounds used were: Citalopram (Tocris Bioscience 1427). JHW007 (Tocris Bioscience 4351). Nomifensine (Abcam abl46004). Test Compounds for the study were: racemic methylone HCl; (R)-methylone HCl; (S)-methylone HCl.

For membrane preparations, rat brains were dissected, tissue was added to ice-cold lysis buffer (50 mM Tris HCl; 5 mM MgCl2; 5 mM EDTA; protease inhibitor cocktail) and homogenized. The homogenate was centrifuged at 100×g for 2 minutes and the supernatant divided into polypropylene Eppendorf tubes. The supernatants were centrifuged at 17,000×g for 10 minutes at 4° C. to re-pellet the cell lysate. The pellet was resuspended in fresh wash buffer (50 mM Tris-HCl; 5 mM MgCl2; 5 mM EDTA) and centrifuged a third time at 17,000×g for 10 minutes at 4° C. The pellet was resuspended in wash buffer containing 10% sucrose as a cryoprotectant, divided into aliquots (0.3 mL) and stored at −80° C. A sample of the homogenate was analyzed for protein content using the MERCK® BCA assay. On the day of the assay, the membrane preparation was thawed and the pellet resuspended in final assay buffer.

Competition binding assays were carried out in 96-well polypropylene plates in a final volume of 250 μL per well. To each well was added 150 μL membranes, 50 μL of test compound, non-specific compound or buffer, and 50 μL radioligand solution in buffer. The plate was incubated at 30° C. for 90 minutes with gentle agitation. The incubation was stopped by vacuum filtration onto presoaked (wash buffer with PEI) GF/C filters using a 96-well FILTERMATE™ harvester, followed by 5 washes with ice-cold wash buffer. Filters were then dried under a warm air stream, sealed in polyethylene, scintillation cocktail added, and the radioactivity counted in a WALLAC® TriLux 1450 MicroBeta counter. For each concentration of drug, non-specific binding was subtracted from total binding to give specific binding. Data was fitted using the non-linear curve fitting routines in PRISM® (Graphpad Software Inc) to determine IC₅₀. Kᵢ was subsequently calculated using the ChengPrusoff equation.

### Results:

TABLE 15 shows a summary of the IC₅₀ and inhibitor constant values (Kᵢ) for racemic methylone, (R)-methylone and (S)-methylone at SERT, DAT and NET. Overall, results revealed that (S)-methylone was a more potent inhibitor of SERT than racemic methylone, had a comparable effect at DAT and was nearly two-fold less potent than racemic methylone at NET. In contrast, (R)-methylone was approximately three-fold less potent than racemic methylone at SERT and DAT, but comparable at NET. This suggests that (S)-methylone shows a potentially favorable binding profile that achieves (1) more potent affinity for SERT, the site thought to underlie methylone's efficacy, (2) comparable affinity for DAT, and (3) lower affinity for NET, the site which may underlie methylone's cardiovascular effects. In summary, based on their action at the monoamine transporters SERT, DAT and NET, the (S)-methylone stereoisomer has the potential to show the beneficial activity of racemic methylone at lower doses and thus potentially with fewer cardiovascular side effects.

TABLE 15
Summary of Competitive Ligand Binding Studies

| Target transporter | Compound ID | IC₅₀ (μM) | Kᵢ(μM)* |
|---|---|---|---|
| 5-HT | Racemic methylone | 12.2744 | 7.4356 |
| 5-HT | (R)-methylone | 34.7356 | 21.0532 |
| 5-HT | (S)-methylone | 6.5163 | 3.9475 |
| DA | Racemic methylone | 7.1121 | 3.5331 |
| DA | (R)-methylone | 24.7172 | 12.2788 |
| DA | (S)-methylone | 8.3368 | 4.1415 |
| NE | Racemic methylone | 3.0974 | 1.4558 |
| NE | (R)-methylone | 4.6659 | 2.1883 |
| NE | (S)-methylone | 7.1179 | 3.3737 |

### Example 7

### Methylone Enantiomers: Effect on Reuptake Inhibition of Serotonin, Dopamine and Norepinephrine.

To extend the results of the binding study and to determine whether the differential effects of methylone enantiomers on binding to SERT, NET, and DAT had functional effects on neurotransmitter reuptake inhibition, whether methylone enantiomers (R)-methylone or (S)-methylone inhibited reuptake of serotonin (5HT), norepinephrine (NE) and/or dopamine (DA) to the same extent as racemic methylone was investigated.

### Methods:

Materials used were: Neurotransmitters [³H]5-HT (PerkinElmer, NET498001MC) [³H]Dopamine (PerkinElmer, NET673250UC) [³H]Norepinephrine (PerkinElmer, NET377250UC); Test Compounds Racemic methylone HCl (Merck, M-140); (R)-methylone HCl (Pisgah Labs); (S)-methylone HCl (Pisgah Labs); Reference Compounds Citalopram (Tocris Bioscience 1427). JHW007 (Tocris Bioscience 4351). Nomifensine (Abcam abl46004).

Synaptosomes were prepared from Sprague Dawley (200-250g) rat brain regions (hippocampus for NE, striatum for DA and midbrain for 5-HT) using standard protocols. Tissues were dissected, added to sucrose buffer (0.32 M), homogenized with a dounce-homogenizer and centrifuged at 100×g to remove cells and debris. Supernatant was collected and centrifuged 17,000× g for 10 minutes at 4° C. to pellet the synaptosomes. The pellet was resuspended in fresh assay buffer.

Uptake assays were carried out in 96-well plates in a final volume of 250 μL per well. To each well was added 150 μL synaptosomes, 50 μL test, non-specific compound or buffer alone. The plate was incubated at 30° C. for 30 minutes with gentle agitation. 50 μL radiolabeled neurotransmitter in buffer was then added to each well to initiate the uptake. The plate was incubated at 30° C. for a further 5 minutes with gentle agitation. The incubation was stopped by vacuum filtration onto presoaked GF/C filters using a 96-well FilterMate™ harvester, followed by 3 washes with ice-cold wash buffer. Filters were then dried under a warm air stream, sealed in polyethylene, scintillation cocktail added, and the radioactivity counted in a Wallac© TriLux 1450 MicroBeta counter.

For each concentration of drug, non-specific uptake was subtracted from total uptake to give specific uptake. Data was fitted using the non-linear curve fitting routines in Prism® (Graphpad Software Inc) to determine IC₅₀.

### Results:

TABLE 16 shows a summary of the IC₅₀ values for uptake inhibition and ECso values for release for racemic methylone, (R)-methylone and (S)-methylone, respectively, for each of 5HT, DA, and NE. Overall, results were consistent with what was observed in the competitive binding study. Specifically, (S)-methylone is a more potent reuptake inhibitor of serotonin (5HT) and dopamine (DA) and a more potent serotonin releaser compared with racemic methylone and (R)-methylone. Effects on norepinephrine (NE) were consistent across all three compounds. Together with the competitive binding study results, these results lend further support that the (S)-methylone stereoisomer offers greater efficacy via effects on serotonin and less cardiovascular side effects via norepinephrine.

TABLE 16
Summary of Uptake Inhibition and Release Studies with
(R)-methylone, (S)-methylone and Racemic Methylone

| | | Uptake | |
|---|---|---|---|
| | | Inhibition | Release |
| Neurotransmitter | Compound | IC₅₀ (nM) | EC₅₀ (μM) |
| 5-HT | Racemic methylone | 811 | 0.62 |
| 5-HT | (R)-methylone | 7621 | ND |
| 5-HT | (S)-methylone | 395 | 1.18 |
| DA | Racemic methylone | 1429 | 4.79 |
| DA | (R)-methylone | 3436 | 4.77 |
| DA | (S)-methylone | 634 | 3.28 |
| NE | Racemic methylone | 355 | 0.33 |
| NE | (R)-methylone | 242 | 0.191 |
| NE | (S)-methylone | 316 | 0.76 |

### EXAMPLE 8

### Neither Methylone Nor its Stereoisomers Bind to Cardiac Channels in an In Vitro Cardiovascular Safety Screening Test.

An in vitro cardiovascular safety screening test was run to investigate the effects of racemic methylone and two methylone stereoisomers ((R)-methylone and (S)-methylone) on selected ion channels critically linked to cardiovascular function and activity. Antagonism of any of the ion channels in this screen would significantly increases risk of cardiovascular effects.

### Methods:

Electrophysiological assays were conducted to profile three compounds ((R)-methylone, (S)-methylone, or racemic methylone) for activities on the ion channel targets specified below using the Qube electrophysiological platform. Where presented, IC₅₀ values were determined by a non-linear, least squares regression analysis. Reference standards were run as an integral part of each assay to ensure the validity of the results obtained. Results showing an inhibition greater than 50% are considered to represent significant effects of test compounds and listed in the following tables with individual calculation results or calculable IC₅₀.

The automated whole cell patch-clamp (Qube 384) technique was used to record depolarizing currents, hNav1.5 and hCav1.2, and outward potassium currents, hERG in multihole mode. Recombinant HEK-293 cells stably transfected with human NavL.5 cDNA, recombinant HEK293 cell line expressing the human Cavl.2 (L-type voltage gated calcium channel, hCavl.2 alC/02a/a281, and recombinant CHO-K1 cells stably transfected with human hERG cDNA were used separately in each of these assays.

Reference compounds: Tetracaine, Nifedipine, and Verapamil were tested concurrently for hNav1.5, hCav1.2 and hERG, respectively at multiple concentrations to obtain an IC50 value.

Ion Channels tested were Eurofins Panel Part Number: CPROFullQB2DR Voltage-Gated Sodium: HEK-Nav1.5 (Peak), HEK-Nav1.5 (Late, Antagonist) Voltage-Gated Potassium: HEK-Kv4.3/KChIP2, CHO-hERG, CHO-KCNQ1/minK Voltage-Gated Calcium: HEK-Cav1.2 Inward-Rectifying Voltage-Gated Potassium: HEK-Kir2.1.

Methods employed in this study have been developed and validated with reliability and reproducibility by Eurofins. Assays were performed under conditions below. [0217] hNavL.5 Sodium Channel Assay—Qube APC Onset and steady state block of peak Nay1.5 current is measured using a pulse pattern, repeated every 5 sec, consisting of a hyperpolarizing pulse to −120 mV for a 200 ms duration, depolarization to −15 mV amplitude for a 40 ms duration, followed by step to 40 mV for 200 ms and finally a 100 ms ramp (1.2 V/s) to a holding potential of −80 mV. Peak current is measured during the step to −15 mV. [0218] hKv4.3/hKChIP2 Potassium Channel Assay—Qube APC After whole cell configuration is achieved, the cells are held at −80 mV. Onset and steady state block of hKv4.3 current is measured using a pulse pattern from −80 mV to 40 mV amplitude for a 110 ms duration, and finally a 100 ms ramp (1.2 V/s) to −80 mV. This paradigm is delivered once every 5s to monitor the current amplitude. [0219] hCav1.2 (L-type) Calcium Channel Assay—Qube APC After whole cell configuration is achieved, the cells are held at −90 mV. Cavl.2 currents are evoked by a 50 ms pulse to −100 mV followed by a 200 ms pulse to +20 mV before returning to the holding potential of −90 mV. This paradigm is delivered three times every 60s to monitor the current amplitude. [0220] hNav1.5 Late Current Sodium Channel Assay—Qube APC Onset and steady state block of Late Nay1.5 current is measured using a pulse pattern, repeated every 5 sec, consisting of a hyperpolarizing pulse to −120 mV for a 200 ms duration, depolarization to −15 mV amplitude for a 40 ms duration, followed by step to 40 mV for 200 ms and finally a 100 ms ramp (1.2 V/s) to a holding potential of −80 mV. Late current is measured as charge current elicited during the ramp with 50 nM ATXII.

hERG Potassium Channel Assay—Qube APC After whole cell configuration is achieved, the cells are held at −80 mV. Cells are held at this voltage for 50 ms to measure the leak current, which is subtracted from the tail current on-line. The cells are depolarized to +40 mV for 500 ms and then to −80 mV over a 100 ms ramp to elicit the hERG tail current. This paradigm is delivered once every 8s to monitor the current amplitude.

hKCNQ1/hminK Potassium Channel Assay—Qube APC After whole cell configuration is achieved, the cells are held at −80 mV. KCNQ1/minK currents are evoked by a 1000 ms pulse from −80 mV to 60 mV followed by a ramp from 60 mV to −80 mV over 115 ms with the outward peak currents measured upon depolarization of the cell membrane. This paradigm is delivered once every 15s to monitor the current amplitude. [0223] hKir2.1 Potassium Channel Assay—Qube APC After whole cell configuration is achieved, the cells are held at −30 mV. Kir2.1 currents are evoked by a single 500 ms pulse to -120 mV before returning to the holding potential of −30 mV. This paradigm is delivered once every 20s to monitor the current amplitude.

### Results:

As shown in TABLE 17, the results from the current study demonstrated that neither stereoisomer ((R)-methylone or (S)-methylone) nor racemic methylone had any effect on the cardiac ion channels tested. All control (reference) compounds elicited the expected effects, validating the assay. Together, this demonstrates that methylone and its enantiomers do not directly inhibit the activity of cardiac channels.

TABLE 17
Estimated IC₅₀ Compound Summary

| | | | Estimated | Reference | Estimated |
|---|---|---|---|---|---|
| Compound | Target | Mode | IC₅₀ (μM) | Compound | IC₅₀ (μM) |
| Methylone HCl | Nav1.5 | Antagonist | N/C | Tetracaine | 5.9 |
| (R)-methylone | Nav1.5 | Antagonist | N/C | Tetracaine | 5.9 |
| (S)-methylone | Nav1.5 | Antagonist | N/C | Tetracaine | 5.9 |
| Methylone HCl | Kc4.3/ChIP2 | Antagonist | N/C | Flecainide | 19.5 |
| (R)-methylone | Kc4.3/ChIP2 | Antagonist | N/C | Flecainide | 19.5 |
| (S)-methylone | Kc4.3/ChIP2 | Antagonist | N/C | Flecainide | 19.5 |
| Methylone HCl | Cav1.2 | Antagonist | N/C | Nifedipine | 0.043 |
| (R)-methylone | Cav1.2 | Antagonist | N/C | Nifedipine | 0.043 |
| (S)-methylone | Cav1.2 | Antagonist | N/C | Nifedipine | 0.043 |
| Methylone HCl | Nav1.5 Late Current | Antagonist | N/C | Ranolazine | 54.6 |
| (R)-methylone | Nav1.5 Late Current | Antagonist | N/C | Ranolazine | 54.6 |
| (S)-methylone | Nav1.5 Late Current | Antagonist | N/C | Ranolazine | 54.6 |
| Methylone HCl | hERG | Antagonist | N/C | Verapamil | 0.68 |
| (R)-methylone | hERG | Antagonist | N/C | Verapamil | 0.68 |
| (S)-methylone | hERG | Antagonist | N/C | Verapamil | 0.68 |
| Methylone HCl | KCNQ1/mink | Antagonist | N/C | Chromanol 293B | 45.3 |
| (R)-methylone | KCNQ1/mink | Antagonist | N/C | Chromanol 293B | 45.3 |
| (S)-methylone | KCNQ1/mink | Antagonist | N/C | Chromanol 293B | 45.3 |
| Methylone HCl | Kir2.1 | Antagonist | N/C | Barium Chloride | 14.4 |
| (R)-methylone | Kir2.1 | Antagonist | N/C | Barium Chloride | 14.4 |
| (S)-methylone | Kir2.1 | Antagonist | N/C | Barium Chloride | 14.4 |

*N/C indicates observed mean inhibition was <25%

### EXAMPLE 9

### Assessment of Antidepressant Properties of Methylone Stereoisomers Using the Rat Forced Swim Test (FST).

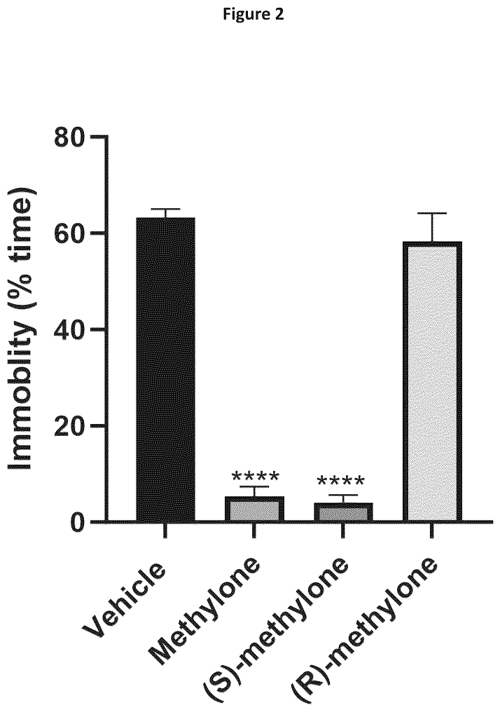

Racemic methylone (10 mg/kg, IP) has a maximal antidepressant-like effect in the rat forced swim test (FST), reducing immobility by nearly 100% compared to vehicle treated controls. To determine whether one enantiomer of methylone could mimic the robust antidepressant-like effect of racemic methylone, rats were treated with a single dose of racemic methylone, (R)-methylone, (S)-methylone (all 10 mg/kg, IP) or vehicle 30 min before testing in the FST. The (S)-methylone enantiomer mimicked the rapid and robust antidepressant-like effect of racemic methylone, whereas (R)-methylone had no effect compared to vehicle-treated animals (FIG. 2). Therefore, all of the activity of methylone appears to be driven by the activity of one enantiomer ((S)-methylone).

### Example 10

### X-Ray Powder Diffraction (Xrpd) Analysis of Synthesized Methylone HCl.

Two GMP Lots 213220 and 22720 of methylone HCL were analyzed assessed and analyzed by X-ray Powder Diffraction (XRPD), NMR and thermogravimetric analysis (TGA).

From a stacked plot of X-ray Powder Diffraction (XRPD) patterns for methylone HCl lots 213220 and 227220, it was readily apparent by eye that the two XRPD patterns do not entirely overlap. Thus, the two lots have distinctly different crystalline phases. Furthermore, no indexing of XPRD was feasible, suggesting that the two lots have a mixture of crystalline forms, and not one form only.

In addition, TGAs for both lots do not show a (sharp or otherwise) melting point, suggesting that no solvates are present. The NMR data for the two lots of methylone also line up exactly, suggesting that (like the TGA results) no solvates are present. Elemental analysis was also conducted on the two lots; none of the Class 1 and 2a were present in them.

Both lots 213220 and 22720 were recrystallized, and re-examining the original material and recrystallized material were examined as above and compared. The XRPD spectra for the recrystallized and original lot for 213220 did not match up. However, the two recrystallized GMP lots were nearly identical to each other, suggesting that they are the same crystalline material. Furthermore, indexing of the two recrystallized lots for 213220 and 227200 suggests a monoclinic structure for each. In addition, matching of the observed XRPD peaks for the recrystallized lot 227220 indicates that the sample consists primarily of a single crystalline form that is comparable to the calculated pattern for the methylone HCl structure (deposition number 819333) obtained from the CCDC (Nycz et al. (2011) Journal of Molecular Structure 1002:10-18). The TGA for recrystallized lot 213220 also does not show any water present.

TABLES 18-21 provide the results of stability studies for methylone HCl lots 213220 and 227220, as well as for capsules containing methylone HCl lot 227220.

TABLE 18

| | | | Lot 213220 (GMP) | | | | |
|---|---|---|---|---|---|---|---|
| | | | | 1 month | 3 months | 3 months | 6 months |
| Test | Method | Test Article | T = 0 | 40° C./75% RH | 40° C./75% RH | 25° C./60% RH | 25° C./60% RH |
| Appearance | Visual | White to off-white, | Conforms | Conforms | Conforms | Conforms | Conforms |
| | | blueish-gray | | | | | |
| | | crystalline solid | | | | | |
| Water | USP<921>; | | 0.1% | 0.13% | 0.1% | 0.1% | 0.31% |
| Content | KF | | | | | | |
| Assay | HPLC | | 97.8% | 99.5% | 99.3% | 99.8% | 98.4% |
| Related | HPLC | 2,3-Methylone | ND | ND | ND | ND | ND |
| Substances | | MDP | ND | ND | BDL | BDL | BDL |
| | | MDPBP | ND | ND | ND | ND | N/A |
| | | Unspecified | ND | ND | RRT 1.64: | RRT 1.64: | BRT |
| | | Impurity | | | 0.08%¹ | 0.08%¹ | |
| | | Total Impurities: | ND | ND | BRT | BRT | BRT |

| | | | Lot 213220 (GMP) | | | |
|---|---|---|---|---|---|---|
| | | | 6 months | 6 months | | |
| | | | Freezer | Refrigerator | 6 months | 9 months |
| Test | Method | Test Article | (−4° C.) | (2-8° C.) | 40° C./75% RH | 25° C./60% RH |
| Appearance | Visual | White to off-white, | Conforms | Conforms | Conforms | Conforms |
| | | blueish-gray | | | | |
| | | crystalline solid | | | | |
| Water | USP<921>; | | 0.25% | 0.31% | 0.46% | 0.22% |
| Content | KF | | | | | |
| Assay | HPLC | | 98.4% | 98.4% | 98.4% | 99.4% |
| Related | HPLC | 2,3-Methylone | ND | ND | ND | ND |
| Substances | | MDP | BDL | BDL | BDL | ND |
| | | MDPBP | N/A | ND | N/A | N/A |
| | | Unspecified | BRT | BRT | BRT | BRT |
| | | Impurity | | | | |
| | | Total Impurities: | BRT | BRT | BRT | BRT |

BDL = below detection limit; BRT = below reporting threshold (<0.05%); GMP = Good Manufacturing Practice; HPLC = high-performance liquid chromatography; KF = Karl Fischer; MDP = 3,4-methylenedioxypropiophenone; MDPBP = 2-bromo-3′,4′-(methylenedioxy)propiophenone; N/A = not available; ND = not detected; NMT = not more than; R&D = research and development; RH = relative humidity; USP = United States Pharmacopeia.
¹Sample preparation error. Preparation amended prior to 6 month timepoints

TABLE 19

| | | | Lot 227220 (GMP) | | | | |
|---|---|---|---|---|---|---|---|
| | | | | 1 month | 3 months | 3 months | 6 months |
| Test | Method | Test Article | T = 0 | 40° C./75% RH | 40° C./75% RH | 25° C./60% RH | 25° C./60% RH |
| Appearance | Visual | White to off- | Conforms | Conforms | Conforms | Conforms | Conforms |
| | | white, blueish- | | | | | |
| | | gray crystalline | | | | | |
| | | solid | | | | | |
| Water | USP<921>; | | 0.20% | 0.19% | 0.20% | 0.20% | 0.29% |
| Content | KF | | | | | | |
| Assay | HPLC | | 97.3% | 98.2% | 100.4% | 98.6% | 97.7% |
| Related | HPLC | 2,3-Methylone | ND | ND | ND | ND | ND |
| Substances | | MDP | ND | ND | BDL | BDL | BDL |
| | | MDPBP | ND | ND | ND | ND | N/A |
| | | Any Unspecified | ND | ND | RRT 0.59: 0.05% | RRT 0.59: 0.05% | RRT 0.58: |
| | | Impurity | | | RRT 1.64: | RRT 1.64: | 0.06% |
| | | | | | 0.12%¹ | 0.14%¹ | |
| | | Total Impurities | ND | ND | 0.05% | 0.05% | 0.06% |

| | | | Lot 227220 (GMP) | | | |
|---|---|---|---|---|---|---|
| | | | 6 months | 6 months | | |
| | | | Freezer | Refrigerator | 6 months | 9 months |
| Test | Method | Test Article | (−4° C.) | (2-8° C.) | 40° C./75% RH | 25° C./60% RH |
| Appearance | Visual | White to off- | Conforms | Conforms | Conforms | Conforms |
| | | white, blueish- | | | | |
| | | gray crystalline | | | | |
| | | solid | | | | |
| Water | USP<921>; | | 0.28% | 0.26% | 0.35% | 0.35% |
| Content | KF | | | | | |
| Assay | HPLC | | 97.9% | 97.7% | 97.4% | 98.8% |
| Related | HPLC | 2,3-Methylone | ND | ND | ND | ND |
| Substances | | MDP | BDL | BDL | ND | ND |
| | | MDPBP | N/A | N/A | N/A | N/A |
| | | Any Unspecified | BRT | BRT | RRT 0.58: | RRT 0.58: |
| | | Impurity | | | 0.05% | 0.06% |
| | | Total Impurities | BRT | BRT | BRT | BRT |

BDL = below detection limit; BRT = below reporting threshold (<0.05%); GMP = Good Manufacturing Practice; HPLC = high-performance liquid chromatography; KF = Karl Fischer; MDP = 3,4-methylenedioxypropiophenone; MDPBP = 2-bromo-3′ ,4′-(methylenedioxy)propiophenone; N/A = not available; ND = not detected; NMT = not more than; R&D = research and development; RH = relative humidity; RRT = relative retention time; USP = United States Pharmacopeia.
¹Sample preparation error. Preparation amended prior to 6 month timepoints

TABLE 20
Stability Test Results for Capsules of Methylone HCl GMP Lot 227220: 40° C./75% RH

| | | | Lot 227220-22342 (GMP) Capsules | |
|---|---|---|---|---|
| Test | Method | Test Article | 1 month | 3 months |
| Appearance | Visual | White Opaque capsules | Conforms | Conforms |
| | | containing white to | | |
| | | off-white, bluish-grey, | | |
| | | crystalline powder | | |
| Proper Closure | Visual & Physical | | Tightly Closed | Tightly Closed |
| Moisture | KF | | 0.2% | 0.2% |
| Assay | HPLC | | 99.8% | 99.8% |
| Related | HPLC | Any Individual Impurity | RRT 0.58: 0.05% | ND |
| Substances | | Total Impurities | 0.05% | ND |

GMP = Good Manufacturing Practice; HPLC = high-performance liquid chromatography, KF = Karl Fischer; ND = not detected; NMT = not more than; RH = relative humidity; RRT = relative retention time; USP = United States Pharmacopeia.

TABLE 21
Stability Test Results for Capsules of Methylone
HCl GMP Lot 227220; Lot: 25° C./60% RH

| | | | Lot 227220-22342 |
|---|---|---|---|
| | | | (GMP) Capsules |
| Test | Method | Test Article | 3 months |
| Appearance | Visual | White Opaque capsules | Conforms |
| | | containing white to | |
| | | off-white, bluish-grey, | |
| | | crystalline powder | |
| Proper Closure | Visual & Physical | | Tightly Closed |
| Moisture | KF | | 0.3% |
| Assay | HPLC | | 99.8% |
| Related | HPLC | Any Individual Impurity | RRT 0.58: 0.05% |
| Substances | | Total Impurities | 0.05% |

GMP = Good Manufacturing Practice; HPLC = high-performance liquid chromatography; KF = Karl Fischer; ND = not detected; NMT = not more than; RH = relative humidity; USP = United States Pharmacopeia.

### Example 11

Evaluation of Methylone Hcl Samples with X-Ray Powder Diffraction (Xrpd) and Dynamic Vapor Sorption (Dvs).

Two lots of methylone HCl samples were submitted to evaluate the stability of the solid forms under various humidity conditions. Dynamic vapor sorption (DVS) analysis was performed to evaluate the kinetic hygroscopicity and assess if variable humidity XRPD would be necessary, as well as to determine the appropriate humidity condition(s) for such XRPD analysis. Both pre- and post-DVS samples were analyzed by conventional XRPD for solid form confirmation or any potential form changes.

### Results and Discussion

The following table summarizes the sample information and their corresponding XRPD and DVS data files:

| | | | Additional | XRPD | DVS | Post-DVS | Post-DVS |
|---|---|---|---|---|---|---|---|
| Sample | TCL ID | Lot No. | Info. | Data | Data | Sample ID | XRPD ID |
| Methylone | TCL20422 | 213220 | CS: 2793, | RX1- | DVS3.513 | 1389-62-01 | RX5-1672 |
| HCl | | | 125 mg | 32777 | | | |
| | TCL20423 | KRR-R&D | 213220, | RX1- | TTGAKIN.902 | 1389-62-02 | RX5-1673 |
| | | 2022-II-58 | CS: 2925, | 32778 | | | |
| | | | Recrystallized, | | | | |
| | | | 125 mg | | | | |

XRPD analysis of as-received samples

The XRPD pattern for lot 213220 was consistent with the previously obtained XRPD pattern for this lot, as described in the previous Example, which was a mixture of Form A and Form B from patent application WO2023/081403 A1, with Form B being the predominant form. Lot KRR-R&D-2022-II-58 was recrystallized from lot 213220 and exhibited primarily a single crystalline Form A phase, consistent with previously acquired data for this lot.

### DVS

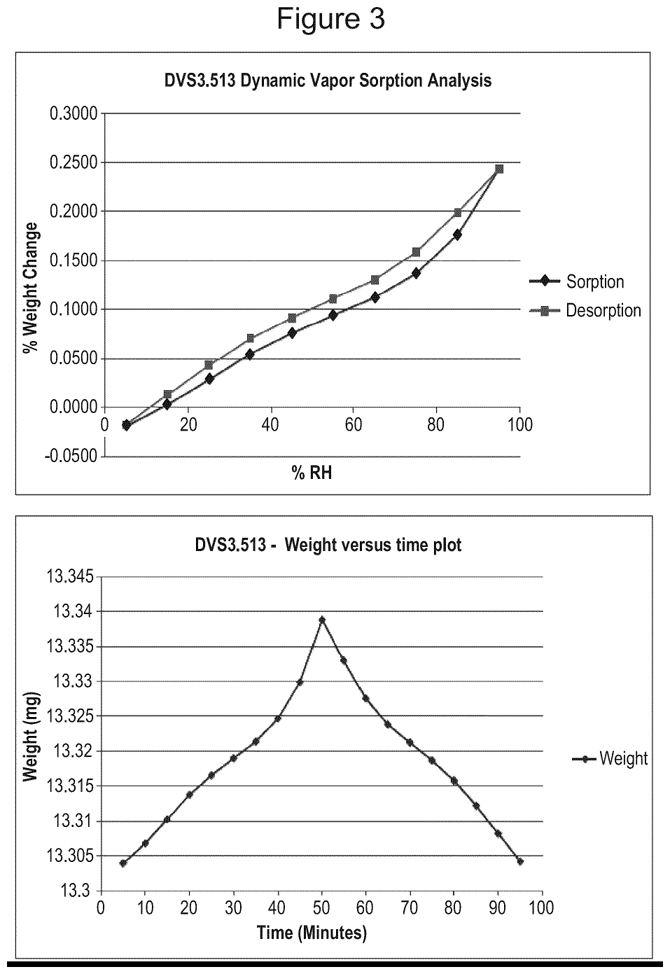

The DVS results for the two samples are summarized in the Table below. The curves depicting percent weight change versus percent relative humidity (% RH) and weight versus time of the samples are shown in FIG. 3 for lot 213220. Both samples exhibited relatively low hygroscopicity with less than 0.3 wt % water sorption between 5% and 95% RH. Upon desorption, the sample of lot 213220 displayed little to no hysteresis and lost almost all of the gained water, while the sample of lot KRR-R&D-2022-II-58 exhibited slight hysteresis with approximately 0.06% of the adsorbed water retained.

| Sample | TCL ID | Results |
|---|---|---|
| Methylone | TCL20422 | Starting weight: 13.306 mg |
| HCl | | 0 019% Wt. loss upon equilibration at 5% R.H |
| | | 0.26% Wt. gain from 5 to 95% RH |
| | | 0.26% Wt. loss from 95% to 5% RH |
| | TCL20423 | Starting weight: 12.158 mg |
| | | 0.034% Wt. gain upon equilibration at 5% RH |
| | | 0.22% Wt. gain from 5 to 95% RH |
| | | 0.15% Wt. loss from 95% to 5% RH |

XRPD analysis of post-DVS samples

The XRPD patterns between the pre- and post-DVS samples were compared for lots 213220 and KRR-R&D-2022-II-58, respectively. No form changes were observed for the post-DVS samples for both lots.

### CONCLUSION

The form stability and kinetic hygroscopicity of two lots of methylone HCl (213220 and KRR-R&D-2022-II-58) were evaluated via XRPD and DVS. XRPD analysis of the pre-DVS samples confirmed that lot 213220 was a mixture of Form A and Form B, with Form B being the predominant form, while lot KRR-R&D-2022-II-58 primarily exhibited a single crystalline Form A phase.

DVS analysis demonstrated that both samples exhibited low hygroscopicity and showed that the material does not absorb or desorb water over 5% to 95% RH, and XRPD analysis of the post-DVS samples revealed no changes in their physical forms. Therefore, there was no need to conduct a variable humidity XRPD study.

### Experimental

### X-ray Powder Diffraction (XRPD)

Each sample was prepared in a silicon low background holder using light manual pressure to keep the sample surface flat and level with the reference surface of the sample holder. The single crystal Si low background holder has a circular recess (10 mm diameter and about 0.2 mm depth) that holds the sample. The Rigaku Smart-Lab diffraction system used was configured for Bragg-Brentano reflection geometry using a line source X-ray beam. The Bragg-Brentano geometry was controlled by passive divergence and receiving slits with the sample itself acting as the focusing component for the optics. Data collection parameters are shown below:

| Parameter | Value | Parameter | Value |
|---|---|---|---|
| Geometry | Bragg-Brentano | Receiving Slit 1 (mm) | 18 |
| Tube Anode | Cu | Receiving Slit 2 (mm) | Open |
| Tube Type | Long Fine Focus | Start Angle (°2θ) | 2 |
| Tube Voltage (kV) | 40 | End Angle (°2θ) | 40 |
| Tube Current (mA) | 44 | Step Size (°2θ) | 0.02 |
| Detector | D/teX Ultra 250 | Scan Speed (°2θ/min) | 6 |
| Monochromatization | Kβ Filter | Spinning (rpm) | 11 |
| Incident Slit (°) | ⅓ | Sample Holder | Low background Si |

### Dynamic Vapor Sorption (DVS)

DVS analyses were carried out using a TA Instruments Q5000 Dynamic Vapor Sorption analyzer. The instrument was calibrated with standard weights and a sodium bromide standard for humidity. Approximately 12-13 mg of each powder sample was loaded into a metal-coated quartz pan for analysis. The samples were analyzed from 5 to 95% RH (adsorption cycle) and from 95 to 5% RH (desorption cycle) in 10% RH steps. The movement from one step to the next occurred either after satisfying the equilibrium criterion of 0.01% weight change in 5 minutes or, if the equilibrium criterion was not met, after 90 minutes. The percent weight change values were calculated using Microsoft EXCEL® 2016.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1.-20. (canceled)

21. A pharmaceutical composition comprising methylone having a purity of greater than 99.7%.

22. The pharmaceutical composition of claim 21, where the composition comprises a pharmaceutically acceptable salt of methylone and/or a substantially pure stereoisomer of methylone, and/or an isotopologue of methylone and/or an isotopomer of methylone, and/or a solvate of methylone, and/or a polymorph of methylone.

23. The pharmaceutical composition of claim 22, wherein the pharmaceutically acceptable salt of methylone is methylone HCl.

24. (canceled)

25. The pharmaceutical composition of claim 21 having a purity of at least about 99.96%.

26. The pharmaceutical composition of claim 21, wherein no impurity is greater than 0.04%.

27. The pharmaceutical composition of claim 21, wherein the composition has no mutagenic impurities.

28. The pharmaceutical composition of claim 21, wherein one or more impurity selected from 2,3-Methylone, 2-bromo-3′,4′-(methylenedioxy)propiophenone (MDPBP) and 3,4-methylenedioxypropiophenone (MDP) is not detectable.

29. The pharmaceutical composition of claim 21 having between 5 mg and 1,000 mg of methylone, a stereoisomer thereof, a pharmaceutically acceptable salt thereof, an isotopologue thereof, an isotopomer thereof, a solvate thereof, and/or a polymorph thereof.

30. The pharmaceutical composition of claim 21, wherein the composition is a room temperature stable composition.

31. The pharmaceutical composition of claim 21, wherein the composition is suitable for use in humans.

32. The pharmaceutical composition of claim 21, wherein the composition conforms to the qualification thresholds set forth in the ICH Q3A and ICH Q3B guidelines.

33.-52. (canceled)

53. A pharmaceutical composition comprising methylone, wherein no impurity is greater than 0.04%.

54. The pharmaceutical composition of claim 53, where the composition comprises a pharmaceutically acceptable salt of methylone and/or a substantially pure stereoisomer of methylone, and/or an isotopologue of methylone and/or an isotopomer of methylone, and/or a solvate of methylone, and/or a polymorph of methylone.

55. The pharmaceutical composition of claim 54, wherein the pharmaceutically acceptable salt of methylone is methylone HCl.

56. The pharmaceutical composition of claim 53 having between 5 mg and 1,000 mg of methylone, a stereoisomer thereof, a pharmaceutically acceptable salt thereof, an isotopologue thereof, an isotopomer thereof, a solvate thereof, and/or a polymorph thereof.

57. The pharmaceutical composition of claim 53, wherein the composition is a room temperature stable composition.

58. The pharmaceutical composition of claim 53, wherein the composition is suitable for use in humans.

59. A pharmaceutical composition comprising methylone, wherein the composition has no mutagenic impurities.

60. The pharmaceutical composition of claim 59, where the composition comprises a pharmaceutically acceptable salt of methylone and/or a substantially pure stereoisomer of methylone, and/or an isotopologue of methylone and/or an isotopomer of methylone, and/or a solvate of methylone, and/or a polymorph of methylone.

61. The pharmaceutical composition of claim 60, wherein the pharmaceutically acceptable salt of methylone is methylone HCl.

62. The pharmaceutical composition of claim 59 having between 5 mg and 1,000 mg of methylone, a stereoisomer thereof, a pharmaceutically acceptable salt thereof, an isotopologue thereof, a solvate thereof, an isotopomer thereof, and/or a polymorph thereof.

63. The pharmaceutical composition of claim 59, wherein the composition is a room temperature stable composition.

64. The pharmaceutical composition of claim 59, wherein the composition is suitable for use in humans.

65. A pharmaceutical composition comprising methylone, wherein the composition conforms to the qualification thresholds set forth in the ICH Q3A and ICH Q3B guidelines.

66. The pharmaceutical composition of claim 65, where the composition comprises a pharmaceutically acceptable salt of methylone and/or a substantially pure stereoisomer of methylone, and/or an isotopologue of methylone and/or an isotopomer of methylone, and/or a solvate of methylone, and/or a polymorph of methylone.

67. The pharmaceutical composition of claim 66, wherein the pharmaceutically acceptable salt of methylone is methylone HCl.

68. The pharmaceutical composition of claim 65 having between 5 mg and 1,000 mg of methylone, a stereoisomer thereof, a pharmaceutically acceptable salt thereof, an isotopologue thereof, an isotopomer thereof, a solvate thereof, and/or a polymorph thereof.

69. The pharmaceutical composition of claim 65, wherein the composition is a room temperature stable composition.

70. The pharmaceutical composition of claim 65, wherein the composition is suitable for use in humans.
